SEQUENCE LISTING (153 sequences)

SEQ ID NO: 1 - protein, 140 aa, Homo sapiens
  PEPTIDE (1)..(140): alpha-Synuclein 1-140
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 134 aa, Homo sapiens
  PEPTIDE (1)..(134): beta-Synuclein 1-134
MDVFMKGLSMAKEGVVAAAEKTKQGVTEAAEKTKEGVLYVGSKTREGVVQGVASVAEKTKEQASHLGGAVFSGAGNIAAATGLVKREEFPTDLKPEEVAQEAAEEPLIEPLMEPEGESYEDPPQEEYQEYEPEA

SEQ ID NO: 3 - protein, 61 aa, Homo sapiens
  PEPTIDE (1)..(61): alpha-Synuclein 80-140
KTVEGAGSIAAATGFVKKDQLGKNEEGAPQEGILEDMPVDPDNEAYEMPSEEGYQDYEPEA

SEQ ID NO: 4 - protein, 56 aa, Homo sapiens
  PEPTIDE (1)..(56): alpha-Synuclein 85-140
AGSIAAATGFVKKDQLGKNEEGAPQEGILEDMPVDPDNEAYEMPSEEGYQDYEPEA

SEQ ID NO: 5 - protein, 50 aa, Homo sapiens
  PEPTIDE (1)..(50): alpha-Synuclein 91-140
ATGFVKKDQLGKNEEGAPQEGILEDMPVDPDNEAYEMPSEEGYQDYEPEA

SEQ ID NO: 6 - protein, 40 aa, Homo sapiens
  PEPTIDE (1)..(40): alpha-Synuclein 101-140
GKNEEGAPQEGILEDMPVDPDNEAYEMPSEEGYQDYEPEA

SEQ ID NO: 7 - protein, 30 aa, Homo sapiens
  PEPTIDE (1)..(30): alpha-Synuclein 111-140
GILEDMPVDPDNEAYEMPSEEGYQDYEPEA

SEQ ID NO: 8 - protein, 20 aa, Homo sapiens
  PEPTIDE (1)..(20): alpha-Synuclein 121-140
DNEAYEMPSEEGYQDYEPEA

SEQ ID NO: 9 - protein, 15 aa, Homo sapiens
  PEPTIDE (1)..(15)
EMPSEEGYQDYEPEA

SEQ ID NO: 10 - protein, 39 aa, Homo sapiens
  PEPTIDE (1)..(39): alpha-Synuclein 97-135
KDQLGKNEEGAPQEGILEDMPVDPDNEAYEMPSEEGYQD

SEQ ID NO: 11 - protein, 35 aa, Homo sapiens
  PEPTIDE (1)..(35): alpha-Synuclein 101-135
GKNEEGAPQEGILEDMPVDPDNEAYEMPSEEGYQD

SEQ ID NO: 12 - protein, 25 aa, Homo sapiens
  PEPTIDE (1)..(25): alpha-Synuclein 111-135
GILEDMPVDPDNEAYEMPSEEGYQD

SEQ ID NO: 13 - protein, 15 aa, Homo sapiens
  PEPTIDE (1)..(15): alpha-Synuclein 121-135
DNEAYEMPSEEGYQD

SEQ ID NO: 14 - protein, 13 aa, Homo sapiens
  PEPTIDE (1)..(13): alpha-Synuclein 123-135
EAYEMPSEEGYQD

SEQ ID NO: 15 - protein, 10 aa, Homo sapiens
  PEPTIDE (1)..(10): alpha-Synuclein 126-135
EMPSEEGYQD

SEQ ID NO: 16 - protein, 32 aa, Homo sapiens
  PEPTIDE (1)..(32): alpha-Synuclein 101-132
GKNEEGAPQEGILEDMPVDPDNEAYEMPSEEG

SEQ ID NO: 17 - protein, 22 aa, Homo sapiens
  PEPTIDE (1)..(22): alpha-Synuclein 111-132
GILEDMPVDPDNEAYEMPSEEG

SEQ ID NO: 18 - protein, 10 aa, Homo sapiens
  PEPTIDE (1)..(10): alpha-Synuclein 80-89
KTVEGAGSIA

SEQ ID NO: 19 - protein, 10 aa, Homo sapiens
  PEPTIDE (1)..(10): alpha-Synuclein 81-90
TVEGAGSIAA

SEQ ID NO: 20 - protein, 10 aa, Homo sapiens
  PEPTIDE (1)..(10): alpha-Synuclein 82-91
VEGAGSIAAA

SEQ ID NO: 21 - protein, 10 aa, Homo sapiens
  PEPTIDE (1)..(10): alpha-Synuclein 83-92
EGAGSIAAAT

SEQ ID NO: 22 - protein, 10 aa, Homo sapiens
  PEPTIDE (1)..(10): alpha-Synuclein 84-93
GAGSIAAATG

SEQ ID NO: 23 - protein, 10 aa, Homo sapiens
  PEPTIDE (1)..(10): alpha-Synuclein 85-94
AGSIAAATGF

SEQ ID NO: 24 - protein, 10 aa, Homo sapiens
  PEPTIDE (1)..(10): alpha-Synuclein 86-95
GSIAAATGFV

SEQ ID NO: 25 - protein, 10 aa, Homo sapiens
  PEPTIDE (1)..(10): alpha-Synuclein 87-96
SIAAATGFVK

SEQ ID NO: 26 - protein, 10 aa, Homo sapiens
  PEPTIDE (1)..(10): alpha-Synuclein 88-97
IAAATGFVKK

SEQ ID NO: 27 - protein, 10 aa, Homo sapiens
  PEPTIDE (1)..(10): alpha-Synuclein 89-98
AAATGFVKKD

SEQ ID NO: 28 - protein, 10 aa, Homo sapiens
  PEPTIDE (1)..(10): alpha-Synuclein 90-99
AATGFVKKDQ

SEQ ID NO: 29 - protein, 10 aa, Homo sapiens
  PEPTIDE (1)..(10): alpha-Synuclein 91-100
ATGFVKKDQL

SEQ ID NO: 30 - protein, 10 aa, Homo sapiens
  PEPTIDE (1)..(10): alpha-Synuclein 92-101
TGFVKKDQLG

SEQ ID NO: 31 - protein, 10 aa, Homo sapiens
  PEPTIDE (1)..(10): alpha-Synuclein 93-102
GFVKKDQLGK

SEQ ID NO: 32 - protein, 10 aa, Homo sapiens
  PEPTIDE (1)..(10): alpha-Synuclein 94-103
FVKKDQLGKN

SEQ ID NO: 33 - protein, 10 aa, Homo sapiens
  PEPTIDE (1)..(10): alpha-Synuclein 95-104
VKKDQLGKNE

SEQ ID NO: 34 - protein, 10 aa, Homo sapiens
  PEPTIDE (1)..(10): alpha-Synuclein 96-105
KKDQLGKNEE

SEQ ID NO: 35 - protein, 10 aa, Homo sapiens
  PEPTIDE (1)..(10): alpha-Synuclein 97-106
KDQLGKNEEG

SEQ ID NO: 36 - protein, 10 aa, Homo sapiens
  PEPTIDE (1)..(10): alpha-Synuclein 98-107
DQLGKNEEGA

SEQ ID NO: 37 - protein, 10 aa, Homo sapiens
  PEPTIDE (1)..(10): alpha-Synuclein 99-108
QLGKNEEGAP

SEQ ID NO: 38 - protein, 10 aa, Homo sapiens
  PEPTIDE (1)..(10): alpha-Synuclein 100-109
LGKNEEGAPQ

SEQ ID NO: 39 - protein, 10 aa, Homo sapiens
  PEPTIDE (1)..(10): alpha-Synuclein 101-110
GKNEEGAPQE

SEQ ID NO: 40 - protein, 10 aa, Homo sapiens
  PEPTIDE (1)..(10): alpha-Synuclein 102-111
KNEEGAPQEG

SEQ ID NO: 41 - protein, 10 aa, Homo sapiens
  PEPTIDE (1)..(10): alpha-Synuclein 103-112
NEEGAPQEGI

SEQ ID NO: 42 - protein, 10 aa, Homo sapiens
  PEPTIDE (1)..(10): alpha-Synuclein 104-113
EEGAPQEGIL

SEQ ID NO: 43 - protein, 10 aa, Homo sapiens
  PEPTIDE (1)..(10): alpha-Synuclein 105-114
EGAPQEGILE

SEQ ID NO: 44 - protein, 10 aa, Homo sapiens
  PEPTIDE (1)..(10): alpha-Synuclein 106-115
GAPQEGILED

SEQ ID NO: 45 - protein, 10 aa, Homo sapiens
  PEPTIDE (1)..(10): alpha-Synuclein 107-116
APQEGILEDM

SEQ ID NO: 46 - protein, 10 aa, Homo sapiens
  PEPTIDE (1)..(10): alpha-Synuclein 108-117
PQEGILEDMP

SEQ ID NO: 47 - protein, 10 aa, Homo sapiens
  PEPTIDE (1)..(10): alpha-Synuclein 109-118
QEGILEDMPV

SEQ ID NO: 48 - protein, 10 aa, Homo sapiens
  PEPTIDE (1)..(10): alpha-Synuclein 110-119
EGILEDMPVD

SEQ ID NO: 49 - protein, 10 aa, Homo sapiens
  PEPTIDE (1)..(10): alpha-Synuclein 111-120
GILEDMPVDP

SEQ ID NO: 50 - protein, 10 aa, Homo sapiens
  PEPTIDE (1)..(10): alpha-Synuclein 112-121
ILEDMPVDPD

SEQ ID NO: 51 - protein, 10 aa, Homo sapiens
  PEPTIDE (1)..(10): alpha1-Synuclein 113-122
LEDMPVDPDN

SEQ ID NO: 52 - protein, 10 aa, Homo sapiens
  PEPTIDE (1)..(10): alpha-Synuclein 114-123
EDMPVDPDNE

SEQ ID NO: 53 - protein, 10 aa, Homo sapiens
  PEPTIDE (1)..(10): alpha-Synuclein 115-124
DMPVDPDNEA

SEQ ID NO: 54 - protein, 10 aa, Homo sapiens
  PEPTIDE (1)..(10): alpha-Synuclein 116-125
MPVDPDNEAY

SEQ ID NO: 55 - protein, 10 aa, Homo sapiens
  PEPTIDE (1)..(10): alpha-Synuclein 117-126
PVDPDNEAYE

SEQ ID NO: 56 - protein, 10 aa, Homo sapiens
  PEPTIDE (1)..(10): alpha-Synuclein 118-127
VDPDNEAYEM

SEQ ID NO: 57 - protein, 10 aa, Homo sapiens
  PEPTIDE (1)..(10): alpha-Synuclein 119-128
DPDNEAYEMP

SEQ ID NO: 58 - protein, 10 aa, Homo sapiens
  PEPTIDE (1)..(10): alpha-Synuclein 120-129
PDNEAYEMPS

SEQ ID NO: 59 - protein, 10 aa, Homo sapiens
  PEPTIDE (1)..(10): alpha-Synuclein 121-130
DNEAYEMPSE

SEQ ID NO: 60 - protein, 10 aa, Homo sapiens
  PEPTIDE (1)..(10): alpha-Synuclein 122-131
NEAYEMPSEE

SEQ ID NO: 61 - protein, 10 aa, Homo sapiens
  PEPTIDE (1)..(10): alpha-Synuclein 123-132
EAYEMPSEEG

SEQ ID NO: 62 - protein, 10 aa, Homo sapiens
  PEPTIDE (1)..(10): alpha-Synuclein 124-133
AYEMPSEEGY

SEQ ID NO: 63 - protein, 10 aa, Homo sapiens
  PEPTIDE (1)..(10): alpha-Synuclein 125-134
YEMPSEEGYQ

SEQ ID NO: 64 - protein, 10 aa, Homo sapiens
  PEPTIDE (1)..(10): alpha-Synuclein 126-135
EMPSEEGYQD

SEQ ID NO: 65 - protein, 10 aa, Homo sapiens
  PEPTIDE (1)..(10): alpha-Synuclein 127-136
MPSEEGYQDY

SEQ ID NO: 66 - protein, 10 aa, Homo sapiens
  PEPTIDE (1)..(10): alpha-Synuclein 128-137
PSEEGYQDYE

SEQ ID NO: 67 - protein, 10 aa, Homo sapiens
  PEPTIDE (1)..(10): alpha-Synuclein 129-138
SEEGYQDYEP

SEQ ID NO: 68 - protein, 10 aa, Homo sapiens
  PEPTIDE (1)..(10): alpha-Synuclein 130-139
EEGYQDYEPE

SEQ ID NO: 69 - protein, 10 aa, Homo sapiens
  PEPTIDE (1)..(10): alpha-Synuclein 131-140
EGYQDYEPEA

SEQ ID NO: 70 - protein, 17 aa, Clostridium tetani
  PEPTIDE (1)..(17): Clostridium tetani 1 Th
KKQYIKANSKFIGITEL

SEQ ID NO: 71 - protein, 15 aa, Measles virus
  PEPTIDE (1)..(15): MvF1 Th
LSEIKGVIVHRLEGV

SEQ ID NO: 72 - protein, 24 aa, Bordetella pertussis
  PEPTIDE (1)..(24): Bordetella pertussis Th
GAYARCPNGTRALTVAELRGNAEL

SEQ ID NO: 73 - protein, 17 aa, Clostridium tetani
  PEPTIDE (1)..(17): Clostridium tetani 2 Th
WVRDIIDDFTNESSQKT

SEQ ID NO: 74 - protein, 23 aa, diphtheria bacilli
  PEPTIDE (1)..(23): Diphtheria Th
DSETADNLEKTVAALSILPGHGC

SEQ ID NO: 75 - protein, 21 aa, Plasmodium falciparum
  PEPTIDE (1)..(21): Plasmodium falciparum Th
DHEKKHAKMEKASSVFNVVNS

SEQ ID NO: 76 - protein, 17 aa, Schistosoma mansoni
  PEPTIDE (1)..(17): Schistosoma mansoni Th
KWFKTNAPNGVDEKHRH

SEQ ID NO: 77 - protein, 25 aa, Cholera Toxin
  PEPTIDE (1)..(25): Cholera Toxin Th
ALNIWDRFDVFCTLGATTGYLKGNS

SEQ ID NO: 78 - protein, 15 aa, Measles virus
  PEPTIDE (1)..(15): MvF 2 Th
ISEIKGVIVHKIEGI

SEQ ID NO: 79 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 80 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 81 - protein, 19 aa, Measles virus
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 82 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 83 - protein, 19 aa, Measles virus
  PEPTIDE (1)..(19): MvF 5 Th
ISITEIKGVIVHRIETILF

SEQ ID NO: 84 - protein, 18 aa, Hepatitis B virus
  PEPTIDE (1)..(18): HBsAg 3 Th
KKKIITITRIITIITTID

SEQ ID NO: 85 - protein, 11 aa, Influenza virus
  PEPTIDE (1)..(11): Influenza Matrix protein 1 _1 Th
  PEPTIDE (1)..(11): Influenza Matrix protein 1_1 Th
FVFTLTVPSER

SEQ ID NO: 86 - protein, 15 aa, Influenza virus
  PEPTIDE (1)..(15): Influenza Matrix protein 1_2 Th
SGPLKAEIAQRLEDV

SEQ ID NO: 87 - protein, 9 aa, Influenza virus
  PEPTIDE (1)..(9): Influenza Non-structural protein 1 Th
DRLRRDQKS

SEQ ID NO: 88 - protein, 19 aa, Epstein-Barr virus
  PEPTIDE (1)..(19): EBV BHRF1 Th
AGLTLSLLVICSYLFISRG

SEQ ID NO: 89 - protein, 15 aa, Clostridium tetani
  PEPTIDE (1)..(15): Clostridium tetani TT1 Th
QYIKANSKFIGITEL

SEQ ID NO: 90 - protein, 20 aa, Epstein-Barr virus
  PEPTIDE (1)..(20): EBV EBNA-1 Th
PGPLRESIVCYFMVFLQTHI

SEQ ID NO: 91 - protein, 21 aa, Clostridium tetani
  PEPTIDE (1)..(21): Clostridium tetani  TT2 Th
FNNFTVSFWLRVPKVSASHLE

SEQ ID NO: 92 - protein, 16 aa, Clostridium tetani
  PEPTIDE (1)..(16): Clostridium tetani TT3 Th
KFIIKRYTPNNEIDSF

SEQ ID NO: 93 - protein, 16 aa, Clostridium tetani
  PEPTIDE (1)..(16): Clostridium tetani  TT4 Th
VSIDKFRIFCKALNPK

SEQ ID NO: 94 - protein, 18 aa, Epstein-Barr virus
  PEPTIDE (1)..(18): EBV CP Th
VPGLYSPCRAFFNKEELL

SEQ ID NO: 95 - protein, 14 aa, Human cytomegalovirus
  PEPTIDE (1)..(14): HCMV IE1 Th
DKREMWMACIKELH

SEQ ID NO: 96 - protein, 15 aa, Epstein-Barr virus
  PEPTIDE (1)..(15): EBV GP340 Th
TGHGARTSTEPTTDY

SEQ ID NO: 97 - protein, 13 aa, Epstein-Barr virus
  PEPTIDE (1)..(13): EBV BPLF1 Th
KELKRQYEKKLRQ

SEQ ID NO: 98 - protein, 11 aa, Epstein-Barr virus
  PEPTIDE (1)..(11): EBV EBNA-2 Th
TVFYNIPPMPL

SEQ ID NO: 99 - protein, 38 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(38): alpha-Synuclein 126-140
ISIXEIXXVIVXXIETILFKKKKEMPSEEGYQDYEPEA

SEQ ID NO: 100 - protein, 43 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(43): alpha-Synuclein 121-140
ISIXEIXXVIVXXIETILFKKKKDNEAYEMPSEEGYQDYEPEA

SEQ ID NO: 101 - protein, 53 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(53): alpha-Synuclein 111-140
ISIXEIXXVIVXXIETILFKKKKGILEDMPVDPDNEAYEMPSEEGYQDYEPEA

SEQ ID NO: 102 - protein, 63 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(63): alpha-Synuclein 126-140
ISIXEIXXVIVXXIETILFKKKKGKNEEGAPQEGILEDMPVDPDNEAYEMPSEEGYQDYEPEA

SEQ ID NO: 103 - protein, 63 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(63): alpha-Synuclein 101-140
ISITEIKGVIVHRIETILFKKKKGKNEEGAPQEGILEDMPVDPDNEAYEMPSEEGYQDYEPEA

SEQ ID NO: 104 - protein, 62 aa, Homo sapiens
  PEPTIDE (1)..(18): HBsAg3 Th
  SITE (19)..(19): epsilon-K
  PEPTIDE (19)..(22): epsilon K-KKK as a spacer
  PEPTIDE (23)..(62): alpha-Synuclein 101-140
KKKIITITRIITIITTIDKKKKGKNEEGAPQEGILEDMPVDPDNEAYEMPSEEGYQDYEPEA

SEQ ID NO: 105 - protein, 73 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(73): alpha-Synuclein 91-140
ISIXEIXXVIVXXIETILFKKKKATGFVKKDQLGKNEEGAPQEGILEDMPVDPDNEAYEMPSEEGYQDYEPEA

SEQ ID NO: 106 - protein, 79 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(79): alpha-Synuclein 85-140
ISIXEIXXVIVXXIETILFKKKKAGSIAAATGFVKKDQLGKNEEGAPQEGILEDMPVDPDNEAYEMPSEEGYQDYEPEA

SEQ ID NO: 107 - protein, 38 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(38): alpha-Synuclein 121-135
ISITEIKGVIVHRIETILFKKKKDNEAYEMPSEEGYQD

SEQ ID NO: 108 - protein, 48 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(48): alpha-Synuclein 111-135
ISITEIKGVIVHRIETILFKKKKGILEDMPVDPDNEAYEMPSEEGYQD

SEQ ID NO: 109 - protein, 58 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(58): alpha-Synuclein 101-135
ISITEIKGVIVHRIETILFKKKKGKNEEGAPQEGILEDMPVDPDNEAYEMPSEEGYQD

SEQ ID NO: 110 - protein, 62 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(62): alpha-Synuclein 97-135
ISITEIKGVIVHRIETILFKKKKKDQLGKNEEGAPQEGILEDMPVDPDNEAYEMPSEEGYQD

SEQ ID NO: 111 - protein, 36 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(36): alpha-Synuclein 123-135
ISITEIKGVIVHRIETILFKKKKEAYEMPSEEGYQD

SEQ ID NO: 112 - protein, 33 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(33): alpha-Synuclein 126-135
ISITEIKGVIVHRIETILFKKKKEMPSEEGYQD

SEQ ID NO: 113 - protein, 45 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(45): alpha-Synuclein 111-132
ISITEIKGVIVHRIETILFKKKKGILEDMPVDPDNEAYEMPSEEG

SEQ ID NO: 114 - protein, 55 aa, Homo sapiens
  PEPTIDE (1)..(19): MvF5 Th
  SITE (20)..(20): epsilon-K
  PEPTIDE (20)..(23): epsilon K-KKK as a spacer
  PEPTIDE (24)..(55): alpha-Synuclein 101-132
ISITEIKGVIVHRIETILFKKKKGKNEEGAPQEGILEDMPVDPDNEAYEMPSEEG

SEQ ID NO: 115 - protein, 45 aa, Mus musculus
  PEPTIDE (1)..(19): MvF 5 Th
  SITE (20)..(20): epsilon-K
  PEPTIDE (20)..(23): epsilon K-KKK as a spacer
  PEPTIDE (24)..(45): Mouse alpha-Synuclein 111-132
ISISEIKGVIVHKIETILFKKKKGILEDMPVDPGSEAYEMPSEEG

SEQ ID NO: 116 - protein, 33 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(33): alpha-Synuclein 126-135
ISIXEIXXVIVXXIETILFKKKKEMPSEEGYQD

SEQ ID NO: 117 - protein, 45 aa, Homo sapiens
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon K-KKK as a spacer
  PEPTIDE (24)..(45): alpha-Synuclein 111-132
ISIXEIXXVIVXXIETILFKKKKGILEDMPVDPDNEAYEMPSEEG

SEQ ID NO: 118 - protein, 30 aa, Homo sapiens
  PEPTIDE (1)..(19): MvF5 Th
  SITE (20)..(20): epsilon K as a spacer
  PEPTIDE (21)..(30): alpha-Synuclein 126-135
ISITEIKGVIVHRIETILFKEMPSEEGYQD

SEQ ID NO: 119 - protein, 42 aa, Homo sapiens
  PEPTIDE (1)..(19): MvF5 Th
  SITE (20)..(20): epsilon K as a spacer
  PEPTIDE (21)..(42): alpha-Synuclein 111-132
ISITEIKGVIVHRIETILFKGILEDMPVDPDNEAYEMPSEEG

SEQ ID NO: 120 - protein, 29 aa, Homo sapiens
  PEPTIDE (1)..(18): HBsAg3 Th
  SITE (19)..(19): epsilon K as a spacer
  PEPTIDE (20)..(29): alpha-Synuclein 126-135
KKKIITITRIITIITTIDKEMPSEEGYQD

SEQ ID NO: 121 - protein, 41 aa, Homo sapiens
  PEPTIDE (1)..(18): HBsAg3 Th
  SITE (19)..(19): epsilon K as a spacer
  PEPTIDE (20)..(41): alpha-Synuclein 111-132
KKKIITITRIITIITTIDKGILEDMPVDPDNEAYEMPSEEG

SEQ ID NO: 122 - protein, 40 aa, Homo sapiens
  PEPTIDE (1)..(17): Clostridium tetani1 Th
  SITE (18)..(18): epsilon K as a spacer
  PEPTIDE (19)..(40): alpha-Synuclein 111-132
KKQYIKANSKFIGITELKGILEDMPVDPDNEAYEMPSEEG

SEQ ID NO: 123 - protein, 38 aa, Homo sapiens
  PEPTIDE (1)..(15): MvF1 Th
  SITE (16)..(16): epsilon K as a spacer
  PEPTIDE (17)..(38): alpha-Synuclein 111-132
LSEIKGVIVHRLEGVKGILEDMPVDPDNEAYEMPSEEG

SEQ ID NO: 124 - protein, 47 aa, Homo sapiens
  PEPTIDE (1)..(24): Bordetella pertussis Th
  SITE (25)..(25): epsilon K as a spacer
  PEPTIDE (26)..(47): alpha-Synuclein 111-132
GAYARCPNGTRALTVAELRGNAELKGILEDMPVDPDNEAYEMPSEEG

SEQ ID NO: 125 - protein, 40 aa, Homo sapiens
  PEPTIDE (1)..(17): Clostridium tetani2 Th
  SITE (18)..(18): epsilon K as a spacer
  PEPTIDE (19)..(40): alpha-Synuclein 111-132
WVRDIIDDFTNESSQKTKGILEDMPVDPDNEAYEMPSEEG

SEQ ID NO: 126 - protein, 46 aa, Homo sapiens
  PEPTIDE (1)..(23): Diphtheria Th
  SITE (24)..(24): epsilon K as a spacer
  PEPTIDE (25)..(46): alpha-Synuclein 111-132
DSETADNLEKTVAALSILPGHGCKGILEDMPVDPDNEAYEMPSEEG

SEQ ID NO: 127 - protein, 44 aa, Homo sapiens
  PEPTIDE (1)..(21): Plasmodium falciparum Th
  SITE (22)..(22): epsilon K as a spacer
  PEPTIDE (23)..(44): alpha-Synuclein 111-132
DHEKKHAKMEKASSVFNVVNSKGILEDMPVDPDNEAYEMPSEEG

SEQ ID NO: 128 - protein, 40 aa, Homo sapiens
  PEPTIDE (1)..(17): Schistosoma mansoni Th
  SITE (18)..(18): epsilon K as a spacer
  PEPTIDE (19)..(40): alpha-Synuclein 111-132
KWFKTNAPNGVDEKHRHKGILEDMPVDPDNEAYEMPSEEG

SEQ ID NO: 129 - protein, 48 aa, Homo sapiens
  PEPTIDE (1)..(25): Cholera Toxin Th
  SITE (26)..(26): epsilon K as a spacer
  PEPTIDE (27)..(48): alpha-Synuclein 111-132
ALNIWDRFDVFCTLGATTGYLKGNSKGILEDMPVDPDNEAYEMPSEEG

SEQ ID NO: 130 - protein, 38 aa, Homo sapiens
  PEPTIDE (1)..(15): MvF2 Th
  SITE (16)..(16): epsilon K as a spacer
  PEPTIDE (17)..(38): alpha-Synuclein 111-132
ISEIKGVIVHKIEGIKGILEDMPVDPDNEAYEMPSEEG

SEQ ID NO: 131 - protein, 45 aa, Homo sapiens
  PEPTIDE (1)..(22): KKKMvF3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
  SITE (23)..(23): epsilon K as a spacer
  PEPTIDE (24)..(45): alpha-Synuclein 111-132
KKKISIXEIXXVIVXXIEXILFKGILEDMPVDPDNEAYEMPSEEG

SEQ ID NO: 132 - protein, 41 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
  SITE (19)..(19): epsilon K as a spacer
  PEPTIDE (20)..(41): alpha-Synuclein 111-132
XXXXXXXTXXXTXPXSXXKGILEDMPVDPDNEAYEMPSEEG

SEQ ID NO: 133 - protein, 41 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
  SITE (19)..(19): epsilon K as a spacer
  PEPTIDE (20)..(41): alpha-Synuclein 111-132
KKKXXXXTRIXTIXXXXDKGILEDMPVDPDNEAYEMPSEEG

SEQ ID NO: 134 - protein, 34 aa, Homo sapiens
  PEPTIDE (1)..(11): Influenza MP1_1 Th
  SITE (12)..(12): epsilon K as a spacer
  PEPTIDE (13)..(34): alpha-Synuclein 111-132
FVFTLTVPSERKGILEDMPVDPDNEAYEMPSEEG

SEQ ID NO: 135 - protein, 38 aa, Homo sapiens
  PEPTIDE (1)..(15): Influenza MP1_2 Th
  SITE (16)..(16): epsilon K as a spacer
  PEPTIDE (17)..(38): alpha-Synuclein 111-132
SGPLKAEIAQRLEDVKGILEDMPVDPDNEAYEMPSEEG

SEQ ID NO: 136 - protein, 32 aa, Homo sapiens
  PEPTIDE (1)..(9): Influenza NSP1 Th
  SITE (10)..(10): epsilon K as a spacer
  PEPTIDE (11)..(32): alpha-Synuclein 111-132
DRLRRDQKSKGILEDMPVDPDNEAYEMPSEEG

SEQ ID NO: 137 - protein, 42 aa, Homo sapiens
  PEPTIDE (1)..(19): EBV BHRF1 Th
  PEPTIDE (20)..(20): epsilon K as a spacer
  SITE (20)..(20): epsilon K as a spacer
  PEPTIDE (21)..(42): alpha-Synuclein 111-132
AGLTLSLLVICSYLFISRGKGILEDMPVDPDNEAYEMPSEEG

SEQ ID NO: 138 - protein, 38 aa, Homo sapiens
  PEPTIDE (1)..(15): Clostridium tetani TT1 Th
  SITE (16)..(16): epsilon K as a spacer
  PEPTIDE (17)..(38): alpha-Synuclein 111-132
QYIKANSKFIGITELKGILEDMPVDPDNEAYEMPSEEG

SEQ ID NO: 139 - protein, 43 aa, Homo sapiens
  PEPTIDE (1)..(20): EBV EBNA-1 Th
  SITE (21)..(21): epsilon K as a spacer
  PEPTIDE (22)..(43): alpha-Synuclein 111-132
PGPLRESIVCYFMVFLQTHIKGILEDMPVDPDNEAYEMPSEEG

SEQ ID NO: 140 - protein, 44 aa, Homo sapiens
  PEPTIDE (1)..(21): Clostridium tetani  TT2 Th
  SITE (22)..(22): epsilon K as a spacer
  PEPTIDE (23)..(44): alpha-Synuclein 111-132
FNNFTVSFWLRVPKVSASHLEKGILEDMPVDPDNEAYEMPSEEG

SEQ ID NO: 141 - protein, 39 aa, Homo sapiens
  PEPTIDE (1)..(16): Clostridium tetani TT3 Th
  SITE (17)..(17): epsilon K as a spacer
  PEPTIDE (18)..(39): alpha-Synuclein 111-132
KFIIKRYTPNNEIDSFKGILEDMPVDPDNEAYEMPSEEG

SEQ ID NO: 142 - protein, 39 aa, Homo sapiens
  PEPTIDE (1)..(16): Clostridium tetani  TT4 Th
  SITE (17)..(17): epsilon K as a spacer
  PEPTIDE (18)..(39): alpha-Synuclein 111-132
VSIDKFRIFCKALNPKKGILEDMPVDPDNEAYEMPSEEG

SEQ ID NO: 143 - protein, 41 aa, Homo sapiens
  PEPTIDE (1)..(18): EBV CP Th
  SITE (19)..(19): epsilon K as a spacer
  PEPTIDE (20)..(41): alpha-Synuclein 111-132
VPGLYSPCRAFFNKEELLKGILEDMPVDPDNEAYEMPSEEG

SEQ ID NO: 144 - protein, 37 aa, Homo sapiens
  PEPTIDE (1)..(14): HCMV IE1 Th
  SITE (15)..(15): epsilon K as a spacer
  PEPTIDE (16)..(37): alpha-Synuclein 111-132
DKREMWMACIKELHKGILEDMPVDPDNEAYEMPSEEG

SEQ ID NO: 145 - protein, 38 aa, Homo sapiens
  PEPTIDE (1)..(15): EBV GP340 Th
  SITE (16)..(16): epsilon K as a spacer
  PEPTIDE (17)..(38): alpha-Synuclein 111-132
TGHGARTSTEPTTDYKGILEDMPVDPDNEAYEMPSEEG

SEQ ID NO: 146 - protein, 36 aa, Homo sapiens
  PEPTIDE (1)..(13): EBV BPLF1 Th
  SITE (14)..(14): epsilon K as a spacer
  PEPTIDE (15)..(36): alpha-Synuclein 111-132
KELKRQYEKKLRQKGILEDMPVDPDNEAYEMPSEEG

SEQ ID NO: 147 - protein, 34 aa, Homo sapiens
  PEPTIDE (1)..(11): EBV EBNA-2 Th
  SITE (12)..(12): epsilon K as a spacer
  PEPTIDE (13)..(34): alpha-Synuclein 111-132
TVFYNIPPMPLKGILEDMPVDPDNEAYEMPSEEG

SEQ ID NO: 148 - protein, 4 aa, Homo sapiens
  PEPTIDE (1)..(4): epsilon K-KKK as spacer
  SITE (1)..(1): epsilon-K
KKKK

SEQ ID NO: 149 - DNA, 31 bp, Homo sapiens
  primer_bind (1)..(31): forward primer sequence for alpha-synuclein
cgggatccgatgtgtttatgaaaggtctgag

SEQ ID NO: 150 - DNA, 31 bp, Homo sapiens
  primer_bind (1)..(31): reverse primer for alpha-synuclein
ggaattccgatgtgtttatgaaaggtctgag

SEQ ID NO: 151 - DNA, 23 bp, Homo sapiens
  primer_bind (1)..(23): Primer sequences for mutant alpha-synuclein
tcatggtgtgaccaccgttgcag

SEQ ID NO: 152 - DNA, 21 bp, Homo sapiens
  primer_bind (1)..(21): reverse primer for mutant alpha-synuclein
accacgccttctttggttttg

SEQ ID NO: 153 - protein, 32 aa, Homo sapiens
  PEPTIDE (1)..(32): beta-synuclein 103-134
AEEPLIEPLMEPEGESYEDPPQEEYQEYEPEA

(19) United States
(12) Patent Application Publication
(10) Pub. No.: US 2021/0138049 A1
(43) Pub. Date: May 13, 2021

(54) PEPTIDE IMMUNOGENS FROM THE C-TERMINAL END OF ALPHA-SYNUCLEIN PROTEIN AND FORMULATIONS THEREOF FOR TREATMENT OF SYNUCLEINOPATHIES

(71) Applicants: UNITED NEUROSCIENCE, Grand Cayman (KY); UNS IP HOLDINGS, LLC, Dallas, TX (US)

(72) Inventor: Chang Yi WANG, Cold Spring Harbor, NY (US)

(21) Appl. No.: 16/623,205

(22) PCT Filed: Jun. 15, 2018
(86) PCT No.: PCT/US18/37938

§ 371 Date: Dec. 16, 2019

Related U.S. Application Data
(60) Provisional application No. 62/521,287, filed on Jun. 16, 2017.

(51) Int. Cl. A61K 39/00

(52) U.S. Cl. CPC A61K 39/0007; A61K 2039/55505; A61K 2039/55561; A61K 2039/6075; A61K 2039/6037; A61K 2039/6068; A61K 2039/55566

(57) ABSTRACT

The present disclosure is directed to alpha-synuclein (α-Syn) peptide immunogen constructs, compositions containing the constructs, antibodies elicited by the constructs, and methods for making and using the constructs and compositions thereof. The disclosed α-Syn peptide immunogen constructs contain a B cell epitope from α-Syn linked to a heterologous T helper cell (Th) epitope directly or through an optional heterologous spacer. The B cell epitope portion of the peptide immunogen constructs contain about 10 to about 25 amino acid residues of α-Syn, corresponding to the sequence from about the Glycine at position 111 (G111) to about the Asparagine at position 135 (D135) of full-length α-Syn. The α-Syn peptide immunogen constructs stimulate the generation of highly specific antibodies that are cross-reactive with the β-sheet of α-Syn as monomers, oligomers, and fibrils, but not the natural α-helix of α-Syn, offering therapeutic immune responses to hosts at risk for synucleinopathies.

The present application is a PCT International Application that claims the benefit of U.S. Provisional Application Ser. No. 62/521,287, filed Jun. 16, 2017, which is incorporated herein by reference in its entirety.

### FIELD OF THE INVENTION

This disclosure relates to peptide immunogen constructs based on the C-terminal end of alpha-synuclein (α-Syn) protein and formulations thereof for treatment of synucleinopathies.

### BACKGROUND OF THE INVENTION

Synuclein proteins (reviewed in website: en.wikipedia.org/wiki/Synuclein) are a family of soluble proteins common to vertebrates that are primarily expressed in neural tissue and in certain tumors. The synuclein family includes three known proteins: alpha-synuclein (reviewed in website: en.wikipedia.org/wiki/Alpha-synuclein), beta-synuclein (website: en.wikipedia.org/wiki/Beta-synuclein), and gamma-synuclein. All synucleins have in common a highly conserved alpha-helical lipid-binding motif with similarity to the class-A2 lipid-binding domains of the exchangeable apolipoproteins. Normal cellular functions have not been determined for any of the synuclein proteins, although some data suggest a role in the regulation of membrane stability and/or turnover.

The full-length alpha-synuclein protein (α-Syn) is a 140 amino acid protein (Accession No. NP_000336) and is encoded by the SNCA gene. At least three isoforms of α-Syn are produced through alternative splicing. The major form is the full-length protein. Other isoforms are α-Syn-126, which lacks residues 41-54 due to loss of exon 3; and α-Syn-112, which lacks residue 103-130 due to loss of exon 5.

The primary structure of α-Syn is usually divided into three distinct domains: (1) residues 1-60: an amphipathic N-terminal region dominated by four 11-residue repeats including the consensus sequence KTKEGV that has a structural alpha helix propensity similar to apolipoproteins-binding domains; (2) residues 61-95: a central hydrophobic region which includes the non-amyloid-β component (NAC) region that is involved in protein aggregation; and (3) residues 96-140: a highly acidic and proline-rich region which has no distinct structural propensity. The 35-amino acid α-Syn fragment of the NAC region was discovered to be present with Aβ in an amyloid-enriched fraction. NAC was later shown to be a fragment of its precursor protein, NACP, later determined to be the full-length human homologue of synuclein from the Pacific electric ray (Torpedo californica), now referred to as human α-Syn.

The use of high-resolution ion-mobility mass spectrometry (IMS-MS) on HPLC-purified α-Syn in vitro has shown α-Syn to be autoproteolytic (self-proteolytic), generating a variety of small molecular weight fragments upon incubation. The 14.46 kDa full-length protein was found to generate numerous smaller fragments, including a 12.16 kDa fragment (amino acids 14-133) and a 10.44 kDa fragment (amino acids 40-140) formed by C- and N-terminal truncations as well as a 7.27 kDa fragment (amino acids 72-140). The 7.27 kDa fragment, which contains the majority of the NAC region, has been shown to aggregate considerably faster than full-length α-Syn. It is possible that these autoproteolytic products play a role as intermediates or cofactors in the aggregation of α-Syn.

α-Syn is abundant in the human brain making up as much as 1% of all proteins in the cytosol of the brain and glial cells. α-Syn is widely expressed in the neocortex, hippocampus, dentate gyrus, olfactory bulb, striatum, thalamus and cerebellum. It is also highly expressed in hematopoietic cells including B-, T-, and NK cells as well as monocytes and platelets. Smaller amounts of α-Syn are found in the heart, muscles, and other tissues. In the brain, α-Syn is found mainly at the tips of nerve cells (neurons) in specialized structures called presynaptic terminals. Within these structures, α-Syn interacts with phospholipids and proteins. Presynaptic terminals release chemical messengers, called neurotransmitters, such as dopamine, from compartments known as synaptic vesicles. The release of neurotransmitters relays signals between neurons and is critical for normal brain function, including cognition.

α-Syn in solution is considered to be an intrinsically disordered protein, in that it lacks a single stable 3D structure. It has been shown that α-Syn significantly interacts with tubulin, and that α-Syn may have activity as a potential microtubule-associated protein, like tau. α-Syn has classically been considered to be an unstructured soluble protein, unmutated α-Syn forms a stably folded tetramer that resists aggregation. Nevertheless, α-Syn can aggregate to form insoluble fibrils in pathological conditions characterized by Lewy bodies. These disorders are known as synucleinopathies (reviewed in website: en.wikipedia.org/wiki/Synucleinopathies).

Synucleinopathies are a diverse group of neurodegenerative disorders that share a common pathologic characteristic: in neuropathologic examinations, characteristic lesions containing abnormal aggregates of insoluble α-Syn are present in selectively vulnerable populations of neurons and glial cells. The most common synucleinopathies include Lewy body disorders (LBDs) like Parkinson's disease (PD), Parkinson's disease with dementia (PDD) and dementia with Lewy bodies (DLB), as well as Multiple System Atrophy (MSA) or Neurodegeneration with Brain Iron Accumulation type I (NBIA Type I). The current treatment options for these diseases include symptomatic medications such as L-dopa, anticholinergic drugs as well as inhibitors of monoamine oxidase. However, all current treatment opportunities only lead to symptomatic alleviation but do not induce a long lasting disease modifying effect in patients.

LBDs are progressive neurodegenerative disorders characterized by tremor, rigidity, bradykinesia and by loss of dopaminergic neurons in the brain. In the case of DLB and PDD, signs also include cognitive impairment. Up to 2% of the population above 60 years of age in western countries develop the typical signs of PD/LBD. It appears that genetic susceptibility and environmental factors are involved in the development of the disease. Patients suffering from this disease develop characteristic intracellular inclusions, called Lewy bodies (LBs), in the cortical and subcortical areas of the brain especially for regions with high content of dopaminergic neurons or neuronal projections. In LBD, α-Syn accumulates in LBs throughout affected brain areas. Additionally, it could be demonstrated that single point mutations as well as duplications or multiplications in the α-Syn gene are associated with rare familial forms of parkinsonism.

Multiple System Atrophy (MSA) is a sporadic neurodegenerative disorder that is characterized by symptoms of L-DOPA-resistant parkinsonism, cerebellar ataxia, and dysautonomia. Patients suffer from multisystem neuronal loss would be affected in various brain areas including striatum, substantia nigra, cerebellum, pons, as well as the inferior olives and the spinal cord. MSA is characterized by α-Syn-positive glial cytoplasmic (GCI) and rare neuronal inclusions throughout the central nervous system.

Other rare disorders, such as various neuroaxonal dystrophies, also have α-Syn pathologies where α-Syn is the primary structural component of Lewy body fibrils. Occasionally, Lewy bodies contain tau protein; however, α-Syn and tau constitute two distinctive subsets of filaments in the same inclusion bodies. α-Syn pathology is also found in both sporadic and familial cases with Alzheimer's disease.

The aggregation mechanism of α-Syn is unclear. Monomeric α-Syn is natively unfolded in solution but can also bind to membranes in an α-helical form. The unfolded monomer can aggregate first into small oligomeric species that can be stabilized by β-sheet-like interactions and then into higher molecular weight insoluble fibrils. α-Syn exists as a mixture of unstructured, alpha-helix, and beta-sheet-rich conformers in equilibrium. Mutations or buffer conditions known to improve aggregation strongly increase the population of the beta conformer, thus suggesting this could be a conformation related to pathogenic aggregation. There is evidence of a structured intermediate rich in beta structure that can be the precursor of aggregation and, ultimately, Lewy bodies.

Several physiological factors may modify α-Syn leading to its formation of aggregates, including (1) phosphorylation by one or more kinases, (2) truncation through protease such as calpains; and (3) nitration through nitric oxide (NO) or other reactive nitrogen species that are present during inflammation. ER-Golgi transport, synaptic vesicles, mitochondria, lysosomes and other proteolytic machinery are some of the proposed cellular targets for α-Syn mediated toxicity due to such aggregation.

Among the strategies for treating synucleinopathies are compounds that inhibit aggregation of α-Syn. It has been shown that the small molecule cuminaldehyde inhibits fibrillation of α-Syn. In addition to small molecule therapies, a recent report suggests that α-Syn aggregates might be targeted by immunotherapy (reviewed by Lee J S and Lee S-J, 2016). However, this report points out several potential issues or problems that exist with developing an α-Syn immunotherapy, including (1) potential interference with normal physiological function of α-Syn; (2) difficulties in delivering an antibody drug to the brain parenchyma; and (3) efficacy of the immunotherapy.

As of this date, there is yet an unmet need to develop site-directed peptide immunogens and formulations thereof for cost effective treatment of patients suffering synucleinopathies.

### REFERENCES

1. “Alpha-synuclein,” Wikipedia, The Free Encyclopedia, website address: en.wikipedia.org/w/index.php?title=Alpha-synuclein&oldid=781366541 (accessed May 30, 2017).
2. “Synucleinopathies,” Wikipedia, The Free Encyclopedia, website address: en.wikipedia.org/w/index.php?title=Synucleinopathies&oldid=686287116 (accessed May 30, 2017).
3. “Beta-synuclein,” Wikipedia, The Free Encyclopedia, website address: en.wikipedia.org/w/index.php?title=Beta-synuclein&oldid=763171134 (accessed May 30, 2017).
4. “Synucleinopathies,” Wikipedia, The Free Encyclopedia, website address: en.wikipedia.org/w/index.php?title=Synucleinopathies&oldid=686287116 (accessed May 30, 2017).
5. LEE, J. S., et al., “Mechanism of Anti-α-Synuclein Immunotherapy”, J Mov Disord.; 9(1):14-19 (2016)
6. TRAGGIAI, E., et al. “An efficient method to make human monoclonal antibodies from memory B cells: potent neutralization of SARS coronavirus”, Nat Med.; 10(8):871-875 (2004)
7. WANG, C., et al. “Versatile Structures of α-Synuclein”, Front Mol Neurosci. 9:48 (2016)

### SUMMARY OF THE INVENTION

The present disclosure is directed to peptide immunogen constructs of the alpha-synuclein protein (α-Syn). The present disclosure is also directed to compositions containing the peptide immunogen constructs, methods of making and using the peptide immunogen constructs, and antibodies produced by the peptide immunogen constructs.

The disclosed peptide immunogen constructs contain a B cell epitope from α-Syn linked to a heterologous T helper cell (Th) epitope directly or through an optional heterologous spacer. The B cell epitope portion of the peptide immunogen constructs contains about 10 to about 25 amino acid residues from the C-terminal region of α-Syn, corresponding to the sequence from about the Glycine at amino acid position 111 (G111) to about the Asparagine at amino acid position 135 (D135) of full-length α-Syn (SEQ ID NO: 1). The heterologous Th epitope portion of the peptide immunogen constructs are derived from amino acid sequences derived from pathogenic proteins. The B cell epitope and Th epitope portions of the peptide immunogen constructs act together when administered to a host to stimulate the generation of antibodies that specifically recognize and bind to the α-Syn B cell epitope portion of the constructs.

In some embodiments, the α-Syn peptide immunogen construct comprises: (a) a B cell epitope comprising about 10 to about 25 amino acid residues from a C-terminal fragment of α-Syn corresponding to about amino acid G111 to about amino acid D135 of SEQ ID NO: 1; (b) a T helper epitope comprising an amino acid sequence selected from the group consisting of SEQ ID NOs: 70-98; and (c) an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 148), wherein the B cell epitope is covalently linked to the T helper epitope directly or through the optional heterologous spacer. In specific embodiments, the α-Syn peptide immunogen construct comprises an amino acid sequence selected from the group consisting of SEQ ID NOs: 107, 108, 111-113, and 115-147.

The present disclosure is also directed to compositions containing the disclosed peptide immunogen constructs, including pharmaceutical compositions. The disclosed pharmaceutical compositions are capable of eliciting an immune response and the production of antibodies against the disclosed peptide immunogen constructs in a host. The disclosed compositions can contain one or a mixture of more than one of the disclosed peptide immunogen constructs. In some embodiments, the compositions contain the disclosed peptide immunogen constructs together with additional components, including carriers, adjuvants, buffers, and other suitable reagents. In certain embodiments, the compositions contain the disclosed peptide immunogen constructs in the form of a stabilized immunostimulatory complex with a CpG oligomer that is optionally supplemented with an adjuvant.

In some embodiments, the compositions comprise an α-Syn peptide immunogen construct comprises an amino acid sequence selected from the group consisting of SEQ ID NOs: 107, 108, 111-113, 115-147. In certain embodiments, the composition is a pharmaceutical composition comprising an α-Syn peptide immunogen construct selected from the group consisting of SEQ ID NOs: 107, 108, 111-113, 115-147 and a pharmaceutically acceptable carrier or adjuvant.

The present disclosure is also directed to antibodies that are produced by a host that is immunized with the disclosed peptide immunogen constructs. The disclosed antibodies specifically recognize and bind to the α-Syn B cell epitope portion of the peptide immunogen constructs. The disclosed α-Syn antibodies have an unexpectedly high cross-reactivity to the β-sheet of α-Syn in the form of monomers, oligomers, or fibrils. Based on their unique characteristics and properties, the disclosed antibodies are capable of providing an immunotherapeutic approach to targeting, identifying, and treating synucleinopathies.

In specific embodiments, the antibody or epitope-binding fragment thereof specifically binds to the B cell epitope of the α-Syn peptide immunogen construct selected from the group consisting of SEQ ID NOs: 107, 108, 111-113, 115-147.

The present disclosure is also directed to methods of making and using the disclosed peptide immunogen constructs, antibodies, and compositions. The disclosed methods provide for the low cost manufacture and quality control of peptide immunogen constructs and compositions containing the constructs, which can be used in methods for preventing and treating synopathies.

The present disclosure also includes methods for treating and/or preventing synucleinopathies using the disclosed peptide immunogen constructs and/or antibodies directed against the peptide immunogen constructs. In some embodiments, the methods for treating and/or preventing synucleinopathies including administering to a host a composition containing a disclosed peptide immunogen construct. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to patients with synucleinopathies. The disclosed methods also include dosing regimens, dosage forms, and routes for administering the peptide immunogen constructs to a host at risk for, or with, synucleinopathies.

In various embodiments, methods of using the α-Syn peptide immunogen construct and/or antibodies elicited by the α-Syn peptide immunogen construct are described. In specific embodiments, the methods are for producing antibodies, inhibiting α-Syn aggregation, reducing the amount of α-Syn aggregates, and identifying α-Syn aggregates of different sizes are described. The various methods comprise administering a pharmacologically effective amount of the α-Syn peptide immunogen to a host in need thereof.

### BRIEF DESCRIPTION OF THE DRAWINGS

FIG. 1 is a graph showing the level of in vitro α-Syn aggregation after 6 days in the presence of antibodies directed against the C-terminal end of α-Syn (Samples 1-4) or in the presence of a vehicle control (Sample 5). Specifically, α-Syn aggregation was carried out in the presence of anti-α-Syn antibodies elicited by: α-Syn₁₁₁₋₁₃₂ (Sample 1); α-Syn₁₂₁₋₁₃₅ (Sample 2); α-Syn₁₂₃₋₁₃₅ (Sample 3); α-Syn₁₂₆₋₁₃₅ (Sample 4); or a vehicle control (Sample 5). The level of α-Syn aggregation was measured by Thioflavin-T (ThT) staining of the aggregates. Samples 1-4 were normalized against the vehicle control of Sample 5. The error bars represent the SEM (standard error of the mean) of each replicated studies.

FIG. 2 is a graph showing the level of dissociation of pre-formed in vitro α-Syn aggregates after incubating the aggregates for 3 days in the presence of antibodies directed against the C-terminal end of α-Syn (Samples 1-3) or a preimmune serum control (Sample 4). Specifically, the pre-formed α-Syn aggregates were incubated with anti-α-Syn antibodies elicited by: α-Syn₁₁₁₋₁₃₂ (Sample 1); α-Syn₁₂₆₋₁₃₅ (Sample 2); a combination of antibodies elicited by α-Syn₁₁₁₋₁₃₂ and α-Syn₁₂₆₋₁₃₅ (Sample 3); or a preimmune serum control (Sample 4). The level of α-Syn aggregation was measured by Thioflavin-T (ThT) staining of the aggregates. Samples 1-3 were normalized against the preimmune serum control of Sample 4. The error bars represent the SEM (standard error of the mean) of each replicated studies.

FIG. 3 is a graph showing the levels of α-Syn aggregation and α-Syn disaggregation in α-Syn-overexpressing PC12 cells incubated with nerve growth factor (NGF) in the presence of antibodies directed against the C-terminal end of α-Syn (Samples 1-4) or a vehicle control (Sample 5). Specifically, the PC12 cells were incubated with anti-α-Syn antibodies elicited by: α-Syn₁₁₁₋₁₃₂ (Sample 1); α-Syn₁₂₁₋₁₃₅ (Sample 2); α-Syn₁₂₃₋₁₃₅ (Sample 3); α-Syn₁₂₆₋₁₃₅ (Sample 4); or a vehicle control (Sample 5). Samples 1-4 were normalized against the vehicle control of Sample 5. The error bars represent the SD (standard deviation) of each triplicated studies.

FIG. 4 is a graph showing the levels of α-Syn aggregate-mediated release of TNF-α and IL-6 from cells incubated in the presence of antibodies directed against the C-terminal end of α-Syn (Samples 1-4) or a vehicle control (Sample 5). Specifically, microglia cells were incubated with anti-α-Syn antibodies elicited by: α-Syn₁₁₁₋₁₃₂ (Sample 1); α-Syn₁₂₁₋₁₃₅ (Sample 2); α-Syn₁₂₃₋₁₃₅ (Sample 3); α-Syn₁₂₆₋₁₃₅ (Sample 4); or a vehicle control (Sample 5). Samples 1-4 were normalized against the vehicle control of Sample 5. The error bars represent the SD (standard deviation) of each triplicated studies.

FIGS. 5A-5C are graphs that illustrate the effect of anti-α-Syn antibodies in an in vitro neurodegeneration model with exogenous, pre-formed α-Syn aggregates in NGF-induced neuronal-differentiated PC12 cells. FIG. 5A evaluates the neurite length of PC12 cells treated with NGF alone (dark solid line); NGF with exogenous pre-formed α-Syn aggregates (dotted line); NGF with preimmune sera (light solid line); and NGF with both exogenous pre-formed α-Syn aggregates and preimmune sera (dashed line). FIG. 5B evaluates the neurite length of PC12 cells treated with NGF along with vehicle (dark solid line); NGF with exogenous pre-formed α-Syn aggregates (dotted line); NGF with anti-α-Syn antibodies elicited by α-Syn₁₁₁₋₁₃₂ (SEQ ID NO:113) (light solid line); and NGF with both exogenous pre-formed α-Syn aggregates and anti-α-Syn antibodies elicited by α-Syn₁₁₁₋₁₃₂ (SEQ ID NO:113) (dashed line). FIG. 5C evaluates the neurite length of PC12 cells treated with NGF alone with vehicle (dark solid line); NGF with exogenous pre-formed α-Syn aggregates (dotted line); NGF with anti-α-Syn antibodies elicited by α-Syn₁₂₆₋₁₃₅ (SEQ ID NO:112) (light solid line); and NGF with both exogenous pre-formed α-Syn aggregates and anti-α-Syn antibodies elicited by α-Syn₁₂₆₋₁₃₅ (SEQ ID NO:112) (dashed line).

FIGS. 6A-6B are graphs that illustrate the effect of anti-α-Syn antibodies on cell number and neurite length in an in vitro neurodegeneration model using NGF-induced neuronal-differentiation wild-type α-Syn-overexpressing PC12 cells. Cells were treated with a vehicle control (Sample 1); anti-α-Syn antibodies elicited by α-Syn₁₀₁₋₁₃₂ (Sample 2), α-Syn₁₁₁₋₁₃₂ (Sample 3), α-Syn₁₂₁₋₁₃₅ (Sample 4), α-Syn₁₂₃₋₁₃₅ (Sample 5), α-Syn₁₂₆₋₁₃₅ (Sample 6), a combination of anti-α-Syn antibodies elicited by α-Syn₁₁₁₋₁₃₂ and α-Syn₁₂₆₋₁₃₅ (Sample 7); or a preimmune serum control (Sample 8). FIG. 6A evaluates each sample's respective protective effects on restoring the number of PC12 cells. FIG. 6B evaluates the neurite length of the cells treated with each sample. Samples 1-8 were normalized to NGF-induced neuronal-differentiated wild-type PC12 cells. A t-test was used for significance testing (a p-value less than 0.05 was defined as statistically significant and denoted with an asterisk (*)).

FIGS. 7A-7B illustrate the ability of anti-α-Syn antibodies to recognize and bind to α-Syn aggregates of different sizes by Western blot analysis. FIG. 7A is an image of a Western blot that compares a commercially available anti-α-Syn antibody, Syn211 (Lane 1); a preimmune serum control (Lane 2); an anti-α-Syn antibody elicited by Syn₁₁₁₋₁₃₂ (Lane 3); an anti-α-Syn antibody elicited by Syn₁₁₁₋₁₃₅ (Lane 4); an anti-α-Syn antibody elicited by Syn₁₂₁₋₁₃₅ (Lane 5); an anti-α-Syn antibody elicited by Syn₁₂₃₋₁₃₅ (Lane 6); and an anti-α-Syn antibody elicited by α-Syn₁₂₆₋₁₃₅ (Lane 7). FIG. 7B is a bar graph that shows the relative ability of each antibody to bind to α-Syn molecular complexes of various sizes (including monomers, dimers, trimers, tetramers, and oligomers). The chemiluminescent signals of the Western blot bands shown in FIG. 7A were quantified and reported in the bar graph of FIG. 7B.

FIGS. 8A-8C are dot blot images that illustrate that the antibodies directed against the C-terminal end of α-Syn only recognize and bind to different species of α-Syn (i.e., the α-helix monomers, β-sheet monomers, β-sheet oligomers and β-sheet fibrils) and not to the same species of other amyloidogenic proteins (i.e., Aβ1-42 and Tau441). FIG. 8A is a control sample showing that antibodies purified from preimmune serum from guinea pigs revealed no detectable level of any to all the protein species assayed. FIG. 8B evaluates the ability of an anti-α-Syn antibody elicited by α-Syn₁₁₁₋₁₃₂ (SEQ ID NO:113) to recognize and bind to different species of α-Syn, Aβ1-42, and Tau441 proteins. FIG. 8C evaluates the ability of an anti-α-Syn antibody elicited by α-Syn₁₂₆₋₁₃₅ (SEQ ID NO:112) to recognize and bind to different species of α-Syn, Aβ1-42, and Tau441 proteins.

FIG. 9 is a table that summarizes the relative binding affinities of antibodies directed against the C-terminal end of α-Syn to intracellular α-Syn in various PC12 cell lines, as measured by positive signals in an immunocytochemistry (ICC) study. Specifically, the relative binding affinities of anti-α-Syn antibodies elicited by α-Syn₁₁₁₋₁₃₂, α-Syn₁₂₁₋₁₃₅, α-Syn₁₂₆₋₁₃₅, or a preimmune serum control sample were evaluated in parental PC12 cells, mock-controlled PC12 cells, wild-type α-Syn-overexpressing PC12 cells, and A53T mutated α-Syn-overexpressing PC12 cells upon NGF treatment.

FIGS. 10A-10C illustrate that the antibodies directed against the C-terminal end of α-Syn only bind to α-Syn in the PD brain sections and not in healthy brain sections FIG. 10A shows the α-Syn peptide immunogen constructs-elicited α-Syn antibodies and the preimmune antibodies showed no detected immunoreactivity on a panel of normal human tissues including the brain sections. FIG. 10B shows the immunoreactivity of antibodies directed against the α-Syn aggregates in the PD thalamus sections as indicated by arrow head. FIG. 10C is a table reporting the immunoreactivity of antibodies directed against the C-terminal end of α-Syn and a preimmune serum control to α-Syn aggregates in the PD and also healthy brain sections, as determined by counting the positive stains under microscopical observation.

FIGS. 11A-11B are graphs showing the level of anti-α-Syn IgG in the serum of PD mouse models after three immunizations with adjuvant alone (open circle) or peptide immunogens containing α-Syn₁₁₁₋₁₃₂ (open square); α-Syn₁₂₆₋₁₃₅ (closed circle); or a combination of α-Syn₁₁₁₋₁₃₂ and α-Syn₁₂₆₋₁₃₅ (closed square). FIG. 11A shows the IgG levels in an MPP⁺ induced mouse model. FIG. 11B shows the IgG levels in a fibrillar α-Syn-inoculated mouse model.

FIGS. 12A-12B are graphs showing the level of α-Syn in the peripheral circulation of the PD mouse models after three immunizations with adjuvant alone (open circle) or peptide immunogens containing α-Syn₁₁₁₋₁₃₂ (open square); α-Syn₁₂₆₋₁₃₅ (closed circle); or a combination of α-Syn₁₁₁₋₁₃₂ and α-Syn₁₂₆₋₁₃₅ (closed square). FIG. 12A shows α-Syn levels in an MPP⁺ induced mouse model. FIG. 12B shows α-Syn levels in an fibrillar α-Syn-inoculated mouse model.

FIGS. 13A-13B show the level of oligomeric α-Syn in brain samples of an untreated healthy mouse model (lane 1) or PD mouse models (lanes 2-3) given three immunizations with either adjuvant alone (lane 2) or peptide immunogens containing α-Syn₁₁₁₋₁₃₂ (lane 3). Untreated Balb/c mice represent the healthy mouse model, while MPP+ induced mice represent the PD mouse models. FIG. 13A is a Western blot showing the level of oligomeric α-Syn, as well as GAPDH as a protein loading control, in the samples. FIG. 13B is a graph comparing the relative oligomeric α-Syn levels shown in the Western blot of FIG. 13A, after the protein levels were normalized with the GAPDH level, and the ratio of the untreated healthy mouse model lysate was further standardized to a level of 1.00 for comparison.

FIGS. 14A-14G show the level of oligomeric α-Syn and tyrosine hydroxylase in brain samples of an untreated healthy mouse model (lane 1) or PD mouse models (lanes 2-4) given three immunizations with either adjuvant alone (lane 2) or peptide immunogens containing α-Syn₁₁₁₋₁₃₂ (lane 3); or α-Syn₁₂₆₋₁₃₅ (lane 4). Untreated FVB mice represent the healthy mouse model, while fibrillar α-Syn inoculated mice represent the PD mouse models. FIG. 14A is a Western blot showing the level of oligomeric α-Syn and tyrosine hydroxylase, as well as GAPDH as a protein loading control, in lysates of the substantia nigra of the ipsilateral side. FIG. 14B is a graph comparing the relative oligomeric α-Syn levels shown in the Western blot of FIG. 14A, after the protein levels were normalized with the GAPDH level. FIG. 14C is a graph comparing the relative tyrosine hydroxylase protein levels shown in the Western blot of FIG. 14A, after the protein levels were normalized with the GAPDH level. FIG. 14D is a Western blot showing the level of oligomeric α-Syn, as well as GAPDH as a protein loading control, in lysates of the striatum of the ipsilateral side. FIG. 14E is a graph comparing the relative oligomeric α-Syn levels shown in the Western blot of FIG. 14C, after the protein levels were normalized with the GAPDH level. FIG. 14F is a Western blot showing the level of oligomeric α-Syn, as well as GAPDH as a protein loading control, in lysates of the striatum of the contralateral side. FIG. 14G is a graph comparing the relative oligomeric α-Syn levels shown in the Western blot of FIG. 14E, after the protein levels were normalized with the GAPDH level.

FIGS. 15A-15C are graphs evaluating motor function in mice measured by CatWalk™ XT in an healthy mouse models (lanes 1-2) treated with saline (lane 1) or adjuvant alone (lane 2); or PD mouse models (lanes 3-5) immunized with either adjuvant alone (lane 3) or peptide immunogens containing α-Syn₁₂₆₋₁₃₅ (lane 4) or α-Syn₁₁₁₋₁₃₂ (lane 5). A t-test was used for significance testing (a p-value less than 0.05 was defined as statistically significant and denoted with an asterisk “*”). FIG. 15A evaluates the left hindlimb stand(s) in the treated mice, where untreated FVB mice represent the healthy mouse model and fibrillar α-Syn inoculated mice represent the PD mouse models. FIG. 15B evaluates the run duration(s) in the treated mice, where untreated FVB mice represent the healthy mouse model and fibrillar α-Syn inoculated mice represent the PD mouse models. FIG. 15C evaluates the run duration(s) in the treated mice, where untreated Balb/c mice represent the healthy mouse model, while MPP+ induced mice represent the PD mouse models.

FIGS. 16A-1611. FIG. 16A shows that PD-021514 (α-Syn₈₅₋₁₄₀, wpi 08) recognizes with the highest affinity α-Syn strain fibrils. Good binding to the strain ribbons and fibrils-91 is observed. Poor binding to oligomers and fibrils-65. Poor binding to α-Syn monomer and to fibrils lacking the C-terminal 30 amino acid residues (Fib-110). FIG. 16B shows that PD-021522 (α-Syn₈₅₋₁₄₀, wpi 13) binds to all strains/oligomers, not to monomers. not observe clearly a concentration-dependent increase in the signal. The antibody binds to fibrils lacking the C-terminal 30 amino acid residues (Fib-110). The epitope is therefore not within this region. FIG. 16C shows that PD-100806 (α-Syn₁₂₆₋₁₃₅, wpi 09) binds to all strains, with highest affinity for ribbons. It binds native oligomeric α-Syn with lower efficiency. Nearly no binding to glutaraldehyde, dopamine cross-linked oligomers and to monomeric a-syn is observed. The antibody is probably directed against a-syn 30 C-terminal amino acid residues as it does not bind fibrils lacking the C-terminal 30 amino acid residues (Fib-110). FIG. 16 D shows that the commercial antibody Syn1 (clone 42, BD bioscience) binds to all α-Syn strains and to oligomers, except Glutaraldehyde cross-links. It also binds to monomeric asyn. Its epitope is described to span over residues 91 to 96/99. Consistent with that, it binds fibrils lacking the C-terminal 30 amino acid residues (Fib-110). FIG. 16E shows that PRX002 recognizes with slightly better affinity fibrillar α-Syn compared to monomeric α-Syn. FIG. 16F shows the control for background of antibodies generated in Guinea Pig. FIG. 16G shows the control for background of the antibody Syn1. FIG. 16H shows the control for background of the PRX002.

FIGS. 17A-17D IHC analysis of the specificity of UNS antibodies for α-Syn in the basal ganglia of patients with Dementia with Lewy Bodies (DLB). The average percentage area of α-Syn aggregates stained by each antibody (PD062220, PD062205, PD100806, and NCL-L-ASYN) was determined for a total area of 7.5 mm² in Putamen (FIG. 17A), Internal capsule (FIG. 17B), and Insula cortex (FIG. 17C). Representative microscope images from immunostaining in the putamen with each antibody is shown in FIG. 17D. The UNS antibodies detected a higher percentage area of α-Syn aggregates in the putamen (F(3,7)=1.550, p=0.284 by ANOVA), internal capsule (F(3,7)=1.356, p=0.332 by ANOVA) and insula cortex (F(3,8)=2.050, p=0.195 by ANOVA). P<0.05 (*); P<0.01 (**); P<0.001 (***). Data are shown as Mean+SD (error bars).

FIGS. 18A-18D IHC analysis of the specificity of UNS antibodies for α-Syn in the basal ganglia of patients with Parkinson's Disease (PD). The average percentage area of α-Syn aggregates stained by each antibody (PD062220, PD062205, PD100806, and NCL-L-ASYN) was determined for a total area of 7.5 mm² in Putamen (FIG. 18A), Internal capsule (FIG. 18B), and Insula cortex (FIG. 18C) of three PD cases. Representative microscope images from immunostaining is shown in FIG. 18D for the Putamen. The UNS antibodies detected a higher percentage area of α-Syn aggregates in the putamen (F(3,18)=4.152, p=0.047 by ANOVA), internal capsule (F(3,8)=1.995, p=0.1934 by ANOVA), and insula cortex (F(3,8)=0.4044, p=0.754 by ANOVA). A significantly higher percentage area of α-Syn was detected with PD100806 compared to NCL-L-ASYN (p=0.023 for PD100806 vs NCL-L-ASYN; n=3). P<0.05 (*); P<0.01 (**); P<0.001 (***). One-way ANOVA was followed by Dunnett test. Data are shown as Mean+SD (error bars).

FIGS. 19A-19C: IHC analysis of the specificity of UNS antibodies for α-Syn in the basal ganglia of patients with Multiple Systems Atrophy (MSA). The average percentage area of α-Syn aggregates stained by each antibody (PD062220, PD062205, PD100806, and NCL-L-ASYN) was determined for a total area of 7.5 mm² in Putamen (FIG. 19A) and Internal capsule (FIG. 19B) in three cases of MSA. No pathology was detected in the insula cortex of patients with MSA and hence was not quantified. The UNS antibodies detected a higher percentage area of α-Syn aggregates in the putamen (F(3,8)=1.56, p=0.273 by ANOVA) and internal capsule (F(3,8)=1.126, p=0.395 by ANOVA). Representative microscope images from immunostaining is shown in FIG. 19C for the putamen with each antibody is shown in C. P<0.05 (*); P<0.01 (**); P<0.001 (***). Data are shown as Mean+SD (error bars).

FIGS. 20A-20E IHC analysis of the specificity of UNS antibodies for α-Syn in the midbrain of patients with different synucleinopathies. The average percentage area of α-Syn aggregates stained by each antibody (PD062220, PD062205, PD100806, and NCL-L-ASYN) was determined for a total area of 7.5 mm² in the substantia nigra of patients with PD (FIG. 20A), DLB (FIG. 20B), and MSA (FIG. 20C). The percentage area stained by each antibody was compared to the diagnostic antibody, NCL-L-ASYN. The UNS antibodies detected a higher percentage area of α-Syn aggregates in the substantia nigra of patients with MSA (F(3,8)=0.830, p=0.51 by ANOVA); DLB (F(3,7)=2.493, p=0.144 by ANOVA) and PD (F(3,7)=0.189, p=0.900 by ANOVA). Representative microscope images from immunostaining with each antibody is shown in FIG. 20D (MSA) and FIG. 20E (DLB). P<0.05 (*); P<0.01 (**); P<0.001 (***). Data are shown as Mean+SD (error bars).

FIGS. 21A-21F IHC analysis of the specificity of UNS antibodies for α-Syn in the white and grey matter of Temporal Cortex of patients with different synucleinopathies. The average percentage area of α-Syn aggregates stained by each antibody (PD062220, PD062205, PD100806, and NCL-L-ASYN) was determined for a total area of 7.5 mm² in the Cortical grey matter and subcortical white matter of patients with PD (FIGS. 21A & 21D), DLB (FIGS. 21B & 21E) and MSA (FIGS. 21C & 21F). The percentage area stained by each antibody was compared to the diagnostic antibody, NCL-L-ASYN. P<0.05 (*); P<0.01 (**); P<0.001 (***). One-way ANOVA was followed by Dunnett test. Data are shown as Mean+SD (error bars).

FIGS. 22A-22C IHC analysis of the specificity of UNS antibodies for α-Syn in the cerebellum of patients with different synucleinopathies. The average percentage area of α-Syn aggregates stained by each antibody (PD062220, PD062205, PD100806, and NCL-L-ASYN) was determined for a total area of 7.5 mm² in the cerebellar white matter of patients with PD (FIG. 22A), DLB (FIG. 22B) and MSA (FIG. 22C). The UNS antibodies detected a higher percentage area of α-Syn aggregates in MSA (F(3,8)=0.929, p=0.469 by ANOVA); DLB (F(3,6)=1.426, p=0.325 by ANOVA) and PD (F(3,6)=2.509, p=0.157 by ANOVA). The percentage area stained by each antibody was compared to the diagnostic antibody, NCL-L-ASYN. P<0.05 (*); P<0.01 (**); P<0.001 (***). Data are shown as Mean+SD (error bars).

FIGS. 23A-23B Representative images of immunostaining of the substantia nigra (FIG. 23A) and putamen (FIG. 23B) from non-diseased control patient brains with each antibody. None of the UNS antibodies detected any α-Syn pathology, comparable to the NCL-L-ASYN diagnostic antibody.

FIGS. 24A-24D IHC analysis of the specificity of UNS antibodies for LBs in the Insula Cortex of the basal ganglia of patients with DLB or PD. The average percentage area of immuno-positive LBs detected with each antibody (PD062220, PD062205, PD100806, and NCL-L-ASYN) was determined for a total area of 7.5 mm² in the insula cortex of patients with PD (FIG. 24A), and DLB (FIG. 24B). The percentage area of LBs is presented as a proportion of the total α-Syn detected with each antibody. The UNS antibodies detected a lower proportion of LBs (or a higher proportion of LNs) in the insula cortex of patients with DLB (F(3,7)=0.836, p=0.516 by ANOVA) and PD (F(3,4)=0.913, p=0.510 by ANOVA). The percentage area stained by each antibody was compared to the diagnostic antibody, NCL-L-ASYN. Representative microscope images from immunostaining with each antibody is shown in FIG. 24C (PD) and FIG. 24D (DLB). P<0.05 (*); P<0.01 (**); P<0.001 (***). Data are shown as Mean+SD (error bars).

FIGS. 25A-25D IHC analysis of the specificity of UNS antibodies for LBs in the grey matter of the temporal cortex of patients with DLB or PD. The average percentage area of immuno-positive LBs detected with each antibody (PD062220, PD062205, PD100806, and NCL-L-ASYN) was determined for a total area of 7.5 mm² in the grey matter of patients with PD (FIG. 25A), and DLB (FIG. 25B). The percentage area of LBs is presented as a proportion of the total alpha-synuclein detected with each antibody. The UNS antibodies detected a lower proportion of LBs (or a higher proportion of LNs) in the grey matter of patients with PD (F(2,3)=1.983, p=0.282 by ANOVA) and DLB (F(3,7)=1.906, p=0.217 by ANOVA). The percentage area stained by each antibody was compared to the diagnostic antibody, NCL-L-ASYN. Representative microscope images from immunostaining with each antibody is shown in FIG. 25C (PD) and FIG. 25D (DLB). P<0.05 (*); P<0.01 (**); P<0.001 (***). Data are shown as Mean+SD (error bars).

FIGS. 26A-26B Representative images of immunostaining with UNS antibodies and NCL-L-ASYN in the substantia nigra of the midbrain of patients with DLB (FIG. 26A) of PD (FIG. 26B). There is a higher detection of LNs with UNS antibodies compared to NCL-L-ASYN.

FIG. 27A-27C Cell specific aggregation of α-Syn. Maximum projection overlaid confocal images of α-Syn aggregates from the basal ganglia and midbrain of human cases with PD (FIG. 27A), DLB (FIG. 27B), and MSA (FIG. 27C). α-Syn (PD062205, red) aggregates within neurones (HuD, green) in cases of PD and DLB but not MSA. α-Syn (PD062205) and HuD are labeled in the greyscale figures that are submitted with the application; however, color copies are available upon request. Scale Bars: 10 μM.

FIG. 28A-28C Cell specific aggregation of α-Syn. Maximum projection overlaid confocal images of α-Syn aggregates from human cases of PD (FIG. 28A), DLB (FIG. 28B), and MSA (FIG. 28C). α-Syn (PD062205, red) aggregates Rare located within oligodendrocytes (Olig2, green) in cases of MSA but not PD or DLB. α-Syn (PD062205) and Olig2 are labeled in the greyscale figures that are submitted with the application; however, color copies are available upon request. Scale Bars: 10 μM.

### DETAILED DESCRIPTION OF THE INVENTION

The present disclosure is directed to peptide immunogen constructs of the alpha-synuclein protein (α-Syn). The present disclosure is also directed to compositions containing the peptide immunogen constructs, methods of making and using the peptide immunogen constructs, and antibodies produced by the peptide immunogen constructs.

The disclosed peptide immunogen constructs contain a B cell epitope from α-Syn linked to a heterologous T helper cell (Th) epitope directly or through an optional heterologous spacer. The B cell epitope portion of the peptide immunogen constructs contain about 10 to about 25 amino acid residues from a C-terminal end of α-Syn, corresponding to the sequence from about the Glycine at amino acid position 111 (G111) to about the Asparagine at amino acid position 135 (D135) of full-length α-Syn (SEQ ID NO: 1). The heterologous Th epitope portion of the peptide immunogen constructs are derived from amino acid sequences derived from pathogenic proteins. The B cell epitope and Th epitope portions of the peptide immunogen constructs act together when administered to a host to stimulate the generation of antibodies that specifically recognize and bind to the α-Syn B cell epitope portion of the constructs.

The present disclosure is also directed to compositions containing the disclosed peptide immunogen constructs, including pharmaceutical compositions. The disclosed pharmaceutical compositions are capable of eliciting an immune response and the production of antibodies against the disclosed peptide immunogen constructs in a host. The disclosed compositions can contain one or a mixture of more than one of the disclosed peptide immunogen constructs. In some embodiments, the compositions contain the disclosed peptide immunogen constructs together with additional components, including carriers, adjuvants, buffers, and other suitable reagents. In certain embodiments, the compositions contain the disclosed peptide immunogen constructs in the form of a stabilized immunostimulatory complex with a CpG oligomer that is optionally supplemented with an adjuvant.

The present disclosure is also directed to antibodies that are produced by a host that is immunized with the disclosed peptide immunogen constructs. The disclosed antibodies specifically recognize and bind to the α-Syn B cell epitope portion of the peptide immunogen constructs. The disclosed α-Syn antibodies have an unexpectedly high cross-reactivity to the β-sheet of α-Syn in the form of monomers, oligomers, or fibrils. Based on their unique characteristics and properties, the disclosed antibodies are capable of providing an immunotherapeutic approach to targeting, identifying, and treating synucleinopathies.

The present disclosure is also directed to methods of making and using the disclosed peptide immunogen constructs, antibodies, and compositions. The disclosed methods provide for the low cost manufacture and quality control of peptide immunogen constructs and compositions containing the constructs, which can be used in methods for preventing and treating synopathies.

The present disclosure also includes methods for treating and/or preventing synucleinopathies using the disclosed peptide immunogen constructs and/or antibodies directed against the peptide immunogen constructs. In some embodiments, the methods for treating and/or preventing synucleinopathies including administering to a host a composition containing a disclosed peptide immunogen construct. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to patients with synucleinopathies. The disclosed methods also include dosing regimens, dosage forms, and routes for administering the peptide immunogen constructs to a host at risk for, or with, synucleinopathies.

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### α-Syn Peptide Immunogen Constructs

The present disclosure provides peptide immunogen constructs containing a B cell epitope from α-Syn covalently linked to a heterologous T helper cell (Th) epitope directly or through an optional heterologous spacer.

The phrase “α-Syn peptide immunogen construct”, as used herein, refers to a peptide containing (a) a B cell epitope having about 10 to about 25 amino acid residues from the C-terminal end of α-Syn, corresponding to the sequence from about the glycine at amino acid position 111 (G111) to about the asparagine at amino acid position 135 (D135) of full-length α-Syn (SEQ ID NO: 1); (b) a heterologous Th epitope; and (c) an optional heterologous spacer.

In certain embodiments, the peptide immunogen construct can be represented by the formulae:

(Th)ₘ-(A)ₙ-(α-Syn C-terminal fragment)-X

or

(α-Syn C-terminal fragment)-(A)ₙ-(Th)ₘ-X

wherein

Th is a heterologous T helper epitope;

A is a heterologous spacer;

(α-Syn C-terminal fragment) is a B cell epitope having about 10 to about 25 amino acid residues from the C-terminal end of α-Syn;

X is an α-COOH or α-CONH₂ of an amino acid;

m is from 1 to about 4; and

n is from 0 to about 10.

The various components of the disclosed α-Syn peptide immunogen construct are described below.

a. α-Syn and α-Syn C-Terminal Fragments

The term “α-Syn”, “alpha-synuclein”, “α-synuclein”, and the like, as used herein, refers to (a) the full-length α-Syn protein and/or (b) fragments thereof from any organism that expresses α-Syn. α-Syn features an extreme conformational diversity, which adapts to different conditions in the states of membrane binding, cytosol, and amyloid aggregation and fulfills versatile functions. In some embodiments, the α-Syn protein is from human. In certain embodiments, the full-length human α-Syn protein has 140 amino acids (Accession No. NP_000336) (SEQ ID NO: 1).

The phrase “C-terminal region” or “C-terminal end” of α-Syn, as used herein, refers to any amino acid sequence from the carboxyl-terminal portion of α-Syn. In certain embodiments, the C-terminal region or C-terminal end of α-Syn relates to the amino acid sequence between residues 96-140, or fragments thereof, of α-Syn. The C-terminal region of α-Syn is rich in prolines and negatively charged residues, which are common characteristics found in intrinsically disordered proteins to maintain solubility. The C-terminal region of α-Syn is generally present in a random coil structure due to its low hydrophobicity and high net negative charge. In vitro studies have revealed that α-Syn aggregation can be induced by reduction of pH which neutralizes these negative charges.

The phrase “α-Syn C-terminal fragment” or “B cell epitope from the C-terminal end of α-Syn”, as used herein, refers to a portion of the full-length α-Syn sequence that includes about 10 to about 25 amino acid residues from the C-terminal end of α-Syn, corresponding to the sequence from about the glycine at amino acid position 111 (G111) to about the asparagine at amino acid position 135 (D135) of full-length α-Syn. The α-Syn C-terminal fragment is also referred to herein as the α-Syn G111-D135 peptide and fragments thereof. The various α-Syn C-terminal fragments described herein are referred to by their amino acid positions in relation to the full-length sequence of α-Syn represented by SEQ ID NO: 1.

The amino acid sequences of the α-Syn C-terminal fragments used in the α-Syn peptide immunogen constructs were selected based on a number of design rationales. Several of these rationales include employing an α-Syn peptide sequence that:
(i) does not share significant sequence homology with beta-synuclein (β-Syn) to avoid generating antibodies that are cross-reactive with β-Syn, since β-Syn can bind to α-Syn and prevent its aggregation;
(ii) is devoid of an autologous T helper epitope within α-Syn to prevent autologous T cell activation which could lead to inflammation of the brain resulting in meningococcal encephalitis as previously reported in clinical trials using AN1792 vaccine targeting Aβ1-42 for treatment of Alzheimer's Disease;
(iii) is contained within a region of α-Syn that is susceptible to conformational changes from its native form;
(iv) is non-immunogenic on its own, since it is a self-molecule;
(v) can be rendered immunogenic by a protein carrier or a potent T helper epitope(s);
(vi) when rendered immunogenic and administered to a host:
   (a) elicits high titer antibodies directed against the α-Syn peptide sequence (B cell epitope) and not against the protein carrier or potent T helper epitope(s);
   (b) elicits high titer antibodies that react with the denatured β-sheet of α-Syn, in the form of monomers, oligomers, or fibrils, to allow such antibodies to prevent α-Syn from aggregating, cause any aggregates of α-Syn to disaggregate, and result in the removal of toxic α-Syn oligomers, aggregates, and/or fibrils, thus reducing or preventing α-Syn aggregate load inside the brain;
   (c) does not elicit antibodies that are reactive with native α-Syn, which would pose a high safety concern, since native α-Syn is a major cellular protein with wide tissue distribution.

In consideration of these design rationales, the C-terminal region of α-Syn was chosen as the target for peptide immunogen design. In addition, the C-terminal region of α-Syn was selected because, based on its structural characteristics, this region seemed to be the most susceptible to modulation by antibody or other physical factors compared to other regions of α-Syn.

Assessment of numerous peptide sequences derived from α-Syn, as described further in the Examples, led to the identification and selection of multiple α-Syn peptides that satisfy the design rationales described above. Specifically, the sequences that satisfy the design rationales include peptides having about 10 to about 25 amino acid residues from the C-terminal region of α-Syn, corresponding to the sequence from about the glycine at amino acid position 111 (G111) to about the asparagine at amino acid position 135 (D135) of full-length α-Syn.

In some embodiments, the α-Syn C-terminal fragment is the 25 amino acid α-Syn G111-D135 peptide represented by SEQ ID NO: 12. In other embodiments, the α-Syn C-terminal fragment contains about 10 contiguous amino acids of the α-Syn G111-D135 peptide represented by SEQ ID NO: 12. In certain embodiments, the α-Syn C-terminal fragment contains 10, 11, 12, 13, 14, 15, 16, 17, 18, 19, 20, 21, 22, 23, 24, or 25 contiguous amino acids of the α-Syn G111-D135 peptide represented by SEQ ID NO: 12. In specific embodiments, the α-Syn C-terminal fragment has an amino acid sequence represented by SEQ ID NOs: 12-15, 17, or 49-64, as shown in Table 1.

The α-Syn C-terminal fragment of the present disclosure also includes immunologically functional analogues or homologues of the α-Syn G111-D135 peptide, and fragments thereof. Functional immunological analogues or homologues of α-Syn G111-D135 peptide and fragments thereof include variants that retain substantially the same immunogenicity as the original peptide. Immunologically functional analogues can have a conservative substitution in an amino acid position; a change in overall charge; a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

Immunologically functional analogues include amino acid sequences that comprise conservative substitutions, additions, deletions, or insertions from one to about four amino acid residues that elicit immune responses that are cross-reactive with the α-Syn G111-D135 peptide. The conservative substitutions, additions, and insertions can be accomplished with natural or non-natural amino acids. Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-Aminocaproic acid (Aca; 6-Aminohexanoic acid), hydroxyproline, mercaptopropionic acid (MPA), 3-nitro-tyrosine, pyroglutamic acid, and the like. Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

In one embodiment, the functional immunological analogue of a particular peptide contains the same amino acid sequence as the original peptide and further includes three lysine residues (Lys-Lys-Lys) added to the amino terminus of the α-Syn G111-D135 peptide and fragments thereof B cell epitope peptide. In this embodiment, the inclusion of three lysine residues to the original peptide sequence changes the overall charge of the original peptide, but does not alter the function of the original peptide.

In certain embodiments, a functional analogue of the α-Syn C-terminal fragment has at least 50% identity to the original amino acid sequence. In other embodiments, the functional analogue has at least 80% identity to the original amino acid sequence. In yet other embodiments, the functional analogue has at least 85% identity to the original amino acid sequence. In still other embodiments, the functional analogue has at least 90% or at least 95% identity to the original amino acid sequence.

b. Heterologous T Helper Cell Epitopes (Th Epitopes)

The present disclosure provides peptide immunogen constructs containing a B cell epitope from α-Syn covalently linked to a heterologous T helper cell (Th) epitope directly or through an optional heterologous spacer.

The heterologous Th epitope in the α-Syn peptide immunogen construct enhances the immunogenicity of the α-Syn C-terminal fragment, which facilitates the production of specific high titer antibodies directed against the optimized target B cell epitope (i.e., the α-Syn C-terminal fragment) through rational design.

The term “heterologous”, as used herein, refers to an amino acid sequence that is derived from an amino acid sequence that is not part of, or homologous with, the wild-type sequence of α-Syn. Thus, a heterologous Th epitope is a Th epitope derived from an amino acid sequence that is not naturally found in α-Syn (i.e., the Th epitope is not autologous to α-Syn). Since the Th epitope is heterologous to α-Syn, the natural amino acid sequence of α-Syn is not extended in either the N-terminal or C-terminal directions when the heterologous Th epitope is covalently linked to the α-Syn C-terminal fragment.

The heterologous Th epitope of the present disclosure can be any Th epitope that does not have an amino acid sequence naturally found in α-Syn. The Th epitope can have an amino acid sequence derived from any species (e.g., human, pig, cattle, dog, rat, mouse, guinea pigs, etc.). The Th epitope can also have promiscuous binding motifs to WIC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e. little, if any, of the antibodies generated by the α-Syn peptide immunogen constructs will be directed towards the Th epitope, thus allowing a very focused immune response directed to the targeted B cell epitope of the α-Syn C-terminal fragment.

Th epitopes of the present disclosure include, but are not limited to, amino acid sequences derived from foreign pathogens, as exemplified in Table 2 (SEQ ID NOs: 70-98). Further, Th epitopes include idealized artificial Th epitopes and combinatorial idealized artificial Th epitopes (e.g., SEQ ID NOs: 71 and 78-84). The heterologous Th epitope peptides presented as a combinatorial sequence (e.g., SEQ ID NOs: 79-82), contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 79-82 which are shown in Table 2. Th epitope peptides of the present invention provide broad reactivity and immunogenicity to animals and patients from genetically diverse populations.

α-Syn peptide immunogen constructs comprising Th epitopes are produced simultaneously in a single solid-phase peptide synthesis in tandem with the α-Syn C-terminal fragment. Th epitopes also include immunological analogues of Th epitopes. Immunological Th analogues include immune-enhancing analogs, cross-reactive analogues and segments of any of these Th epitopes that are sufficient to enhance or stimulate an immune response to the α-Syn C-terminal fragments.

Functional immunologically analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the α-Syn C-terminal fragments. Table 2 identifies another variation of a functional analogue for Th epitope peptide. In particular, SEQ ID NOs: 71 and 78 of MvF1 and MvF2 Th are functional analogues of SEQ ID NOs: 81 and 83 of MvF4 and MvF5 in that they differ in the amino acid frame by the deletion (SEQ ID NOs: 71 and 78) or the inclusion (SEQ ID NOs: 81 and 83) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 71, 78, 79, 81, and 83) and from Hepatitis Surface protein HBsAg 1-3 Ths (SEQ ID NOs: 80, 82, and 84).

The Th epitope in the α-Syn peptide immunogen construct can be covalently linked at either N- or C-terminal end of the α-Syn C-terminal peptide. In some embodiments, the Th epitope is covalently linked to the N-terminal end of the α-Syn C-terminal peptide. In other embodiments, the Th epitope is covalently linked to the C-terminal end of the α-Syn C-terminal peptide. In certain embodiments, more than one Th epitope is covalently linked to the α-Syn C-terminal fragment. When more than one Th epitope is linked to the α-Syn C-terminal fragment, each Th epitope can have the same amino acid sequence or different amino acid sequences. In addition, when more than one Th epitope is linked to the α-Syn C-terminal fragment, the Th epitopes can be arranged in any order. For example, the Th epitopes can be consecutively linked to the N-terminal end of the α-Syn C-terminal fragment, or consecutively linked to the C-terminal end of the α-Syn C-terminal fragment, or a Th epitope can be covalently linked to the N-terminal end of the α-Syn C-terminal fragment while a separate Th epitope is covalently linked to the C-terminal end of the α-Syn C-terminal fragment. There is no limitation in the arrangement of the Th epitopes in relation to the α-Syn C-terminal fragment.

In some embodiments, the Th epitope is covalently linked to the α-Syn C-terminal fragment directly. In other embodiments, the Th epitope is covalently linked to the α-Syn C-terminal fragment through a heterologous spacer described in further detail below.

c. Heterologous Spacer

The disclosed α-Syn peptide immunogen constructs optionally contain a heterologous spacer that covalently links the B cell epitope from α-Syn to the heterologous T helper cell (Th) epitope.

As discussed above, the term “heterologous”, refers to an amino acid sequence that is derived from an amino acid sequence that is not part of, or homologous with, the wild-type sequence of α-Syn. Thus, the natural amino acid sequence of α-Syn is not extended in either the N-terminal or C-terminal directions when the heterologous spacer is covalently linked to the B cell epitope from α-Syn because the spacer is heterologous to the α-Syn sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the α-Syn peptide immunogen construct. Structurally, the spacer provides a physical separation of the Th epitope from the B cell epitope of the α-Syn C-terminal fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 148), where Xaa is any amino acid, and preferably aspartic acid.

The spacer can also provide functional features to the α-Syn peptide immunogen construct. For example, the spacer can be designed to change the overall charge of the α-Syn peptide immunogen construct, which can affect the solubility of the peptide immunogen construct. Additionally, changing the overall charge of the α-Syn peptide immunogen construct can affect the ability of the peptide immunogen construct to associate with other compounds and reagents. As discussed in further detail below, the α-Syn peptide immunogen construct can be formed into a stable immunostimulatory complex with a highly charged oligonucleotide, such as CpG oligomers through electrostatic association. The overall charge of the α-Syn peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the α-Syn peptide immunogen construct can be covalently linked at either N- or C-terminal end of the Th epitope and the α-Syn C-terminal peptide. In some embodiments, the spacer is covalently linked to the C-terminal end of the Th epitope and to the N-terminal end of the α-Syn C-terminal peptide. In other embodiments, the spacer is covalently linked to the C-terminal end of the α-Syn C-terminal peptide and to the N-terminal end of the Th epitope. In certain embodiments, more than one spacer can be used, for example, when more than one Th epitope is present in the peptide immunogen construct. When more than one spacer is used, each spacer can be the same as each other or different. Additionally, when more than one Th epitope is present in the peptide immunogen construct, the Th epitopes can be separated with a spacer, which can be the same as, or different from, the spacer used to separate the Th epitope from the B cell epitope. There is no limitation in the arrangement of the spacer in relation to the Th epitope or the α-Syn C-terminal fragment.

In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N)Lys, or ε-N-Lys-Lys-Lys-Lys (SEQ ID NO: 148).

d. Specific Embodiments of the α-Syn Peptide Immunogen Construct

The α-Syn peptide immunogen construct can be represented by the formulae:

(Th)ₘ-(A)ₙ-(α-Syn C-terminal fragment)-X

or

(α-Syn C-terminal fragment)-(A)ₙ-(Th)ₘ-X

wherein

Th is a heterologous T helper epitope;

A is a heterologous spacer;

(α-Syn C-terminal fragment) is a B cell epitope having about 10 to about 25 amino acid residues from the C-terminal end of α-Syn;

X is an α-COOH or α-CONH₂ of an amino acid;

m is from 1 to about 4; and

n is from 0 to about 10.

In certain embodiments, the heterologous Th epitope in the α-Syn peptide immunogen construct has an amino acid sequence selected from any of SEQ ID NOs: 70-98, or combinations thereof, shown in Table 2. In specific embodiments, the Th epitope has an amino acid sequence selected from any of SEQ ID NOs: 78-84. In certain embodiments, the α-Syn peptide immunogen construct contains more than one Th epitope.

In certain embodiments, the optional heterologous spacer is selected from any of Lys-, Gly-, Lys-Lys-Lys-, (α, ε-N)Lys, ε-N-Lys-Lys-Lys-Lys (SEQ ID NO: 148), and combinations thereof. In specific embodiments, the heterologous spacer is ε-N-Lys-Lys-Lys-Lys (SEQ ID NO: 148).

In certain embodiments, the α-Syn C-terminal fragment has about 10 to about 25 amino acid residues from the C-terminal end of α-Syn, corresponding to the sequence from about the glycine at amino acid position 111 (G111) to about the asparagine at amino acid position 135 (D135) of full-length α-Syn. In specific embodiments, the α-Syn C-terminal fragment has an amino acid sequence represented by SEQ ID NOs: 12-15, 17, or 49-64, as shown in Table 1.

In certain embodiments, the α-Syn peptide immunogen construct has an amino acid sequence selected from any of SEQ ID NOs: 107-108, 111-113, and 115-147, as shown in Table 3. In specific embodiments, the α-Syn peptide immunogen construct has an amino acid sequence selected from any of SEQ ID NOs: 107-108 and 111-113.

### Compositions

The present disclosure also provides compositions comprising the disclosed α-Syn peptide immunogen construct.

a. Peptide Compositions

Compositions containing a disclosed α-Syn peptide immunogen construct can be in liquid or solid form. Liquid compositions can include water, buffers, solvents, salts, and/or any other acceptable reagent that does not alter the structural or functional properties of the α-Syn peptide immunogen construct. Peptide compositions can contain one or more of the disclosed α-Syn peptide immunogen constructs.

b. Pharmaceutical Compositions

The present disclosure is also directed to pharmaceutical compositions containing the disclosed α-Syn peptide immunogen construct.

Pharmaceutical compositions can contain carriers and/or other additives in a pharmaceutically acceptable delivery system. Accordingly, pharmaceutical compositions can contain a pharmaceutically effective amount of an α-Syn peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the α-Syn peptide immunogen construct without having any specific antigenic effect itself. Adjuvants used in the pharmaceutical composition can include oils, aluminum salts, virosomes, aluminum phosphate (e.g. ADJU-PHOS®), aluminum hydroxide (e.g. ALHYDROGEL®), liposyn, saponin, squalene, L121, Emulsigen®, monophosphoryl lipid A (MPL), QS21, ISA 35, ISA 206, ISA50V, ISA51, ISA 720, as well as the other adjuvants and emulsifiers.

In some embodiments, the pharmaceutical composition contains Montanide™ ISA 51 (an oil adjuvant composition comprised of vegetable oil and mannide oleate for production of water-in-oil emulsions), Tween® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e. w/o/w) emulsion with Emulsigen or Emulsigen D as the adjuvant.

Pharmaceutical compositions can be formulated as immediate release or for sustained release formulations. Additionally the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the α-Syn peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can be formulated as immediate release or for sustained release formulations. Additionally the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can also formulated in a suitable dosage unit form. In some embodiments, the pharmaceutical composition contains from about 0.5 μg to about 1 mg of the α-Syn peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one α-Syn peptide immunogen construct. A pharmaceutical composition containing a mixture of more than one α-Syn peptide immunogen construct to allow for synergistic enhancement of the immunoefficacy of the constructs. Pharmaceutical compositions containing more than one α-Syn peptide immunogen construct can be more effective in a larger genetic population due to a broad WIC class II coverage thus provide an improved immune response to the α-Syn peptide immunogen constructs.

In some embodiments, the pharmaceutical composition contains an α-Syn peptide immunogen construct selected from SEQ ID NOs: 107-108, 111-113, 115-147, as well as homologues, analogues and/or combinations thereof. In specific embodiments, pharmaceutical compositions contain an α-Syn peptide immunogen construct selected from SEQ ID NOs: 107-108, 111-113, and any combination thereof.

Pharmaceutical compositions containing an α-Syn peptide immunogen construct can be used to elicit an immune response and produce antibodies in a host upon administration.

c. Immunostimulatory Complexes

The present disclosure is also directed to pharmaceutical compositions containing an α-Syn peptide immunogen construct in the form of an immunostimulatory complex with a CpG oligonucleotide. Such immunostimulatory complexes are specifically adapted to act as an adjuvant and as a peptide immunogen stabilizer. The immunostimulatory complexes are in the form of a particulate, which can efficiently present the α-Syn peptide immunogen to the cells of the immune system to produce an immune response. The immunostimulatory complexes may be formulated as a suspension for parenteral administration. The immunostimulatory complexes may also be formulated in the form of w/o emulsions, as a suspension in combination with a mineral salt or with an in-situ gelling polymer for the efficient delivery of the α-Syn peptide immunogen to the cells of the immune system of a host following parenteral administration. The immunostimulatory complexes are capable of producing an immune response toward the β-sheet of α-Syn (e.g. FIGS. 8A, 8B, and 8C of Example 13) with protective/therapeutic benefit.

The stabilized immunostimulatory complex can be formed by complexing an α-Syn peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the α-Syn peptide immunogen construct is designed to contain a cationic portion that is positively charged at a pH in the range of 5.0 to 8.0. The net charge on the cationic portion of the α-Syn peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the α-Syn peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the α-Syn peptide immunogen construct is the heterologous spacer. In certain embodiments, the cationic portion of the α-Syn peptide immunogen construct has a charge of +4 when the spacer sequence is (α, ε-N)Lys, ε-N-Lys-Lys-Lys-Lys (SEQ ID NO: 148).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X¹ is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T.

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

### Antibodies

The present disclosure also provides antibodies elicited by the α-Syn peptide immunogen construct.

The α-Syn C-terminal fragments having about 10 to about 25 amino acid residues from the C-terminal end of α-Syn, corresponding to the sequence from about the glycine at amino acid position 111 (G111) to about the asparagine at amino acid position 135 (D135) of full-length α-Syn are non- or weakly-immunogenic by themselves. However, the disclosed α-Syn peptide immunogen constructs, comprising an α-Syn C-terminal fragment, heterologous Th epitope, and optional heterologous spacer, are capable of eliciting an immune response and the production of antibodies when administered to a host. The design of the α-Syn peptide immunogen constructs can break tolerance to self α-Syn and elicit the production of site-specific antibodies that recognize conformational, not linear, epitopes.

Surprisingly, antibodies produced by the α-Syn peptide immunogen constructs do not bind to the natural alpha-helix of α-Syn monomer in its native form. Instead, the antibodies produced by the α-Syn peptide immunogen constructs recognize and bind to the denatured β-sheet of α-Syn in the forms of monomers, oligomers and fibrils. Additionally, the antibodies produced by the α-Syn peptide immunogen constructs do not bind to similar structures of other amyloidogenic proteins (i.e., Aβ1-42 and Tau441). Thus, the specific design of the α-Syn peptide immunogen construct (comprising an α-Syn C-terminal fragment, heterologous Th epitope, and optional heterologous spacer) appears to have changed the conformation of the versatile α-Syn C-terminal fragments to allow β-sheet like conformation.

Extensive comparisons of antibodies derived from the immune sera from animals immunized with the α-Syn peptide immunogen constructs were made in many functional assays. These comparisons demonstrated the ability of the antibodies to bind to α-Syn in nerve growth factor (NGF) treated PC12 cells with high specificity only to β-sheet monomers and oligomers of α-Syn and not to other species of amyloidogenic proteins (see Example 9).

Antibodies elicited by the α-Syn peptide immunogen constructs surprisingly can prevent aggregation of α-Syn (anti-aggregation activity) and can disassociate preformed α-Syn aggregates (disaggregation activity). Additionally, the antibodies surprisingly can reduce microglial cell induced TNF-alpha and IL6 production, which indicates that these antibodies can effectively reduce α-Syn aggregate or fibril-mediated microglial activation. These antibodies were also found to reduce neurodegeneration triggered both by exogenous α-Syn aggregates and by endogenous α-Syn aggregates in α-Syn-overexpressing cells. Furthermore, such antibodies recognize and bind specifically to pathological α-Syn oligomeric aggregates or fibrils, but do not react to non-pathological α-Syn. Specifically, the antibodies react with Lewy bodies from brain sections taken from patients with Parkinson's disease of alpha Synucleinopathies, but not with normal human tissues.

It was also surprisingly found that two Parkinson mouse models (a MPP+ induced mouse model and a fibrilla α-Syn-inoculated mouse model) that were administered compositions containing the α-Syn peptide immunogen constructs (a) produced antibodies that were highly cross-reactive with the β-sheet of α-Syn, (b) had a reduction in α-Syn serum levels, (c) had a reduction in oligomeric α-Syn levels in the brain, and (d) had a reduction of neuropathology leading to recovery of motor function.

The resulting immune responses from animals immunized with α-Syn peptide immunogen constructs of the present invention demonstrated the ability of the constructs to produce potent site-directed antibodies that are reactive with the denatured β-sheet of α-Syn in the forms of monomers, oligomers and fibrils and not the random coil structure of the C-terminal α-Syn in its native form.

### In Vitro Functional Assays

Antibodies produced by the α-Syn peptide immunogen constructs can be used in in vitro functional assays. These functional assays include, but are not limited to:
(a) inhibition in vitro of recombinant α-Syn aggregation; and disaggregate preformed recombinant α-Syn aggregates (see Example 8);
(b) inhibition in vitro of cellular α-Syn aggregation, and dissociation of preformed α-Syn aggregates inside cells (see Example 9);
(c) reduction of microglial TNF-alpha and IL6 secretion (see Example 10);
(d) reduction of neurodegeneration triggered by exogeneous α-Syn aggregates (see Example 11);
(e) reduction of neurodegeneration in α-Syn overexpressing cells (see Example 12);
(f) in vivo proof of efficacy in fibrillary α-Syn-innoculated- and MPP+-induced-Parkinson's Disease model in mice showing reduction in serum α-Syn level, reduction in oligomeric α-Syn level in brain, reduction in neuropathology and recovery of motor activities (see Example 15).

### Methods

The present disclosure is also directed to methods for making and using the α-Syn peptide immunogen constructs, compositions, and pharmaceutical compositions.

a. Methods for Manufacturing the α-Syn Peptide Immunogen Construct

The α-Syn peptide immunogen constructs of this disclosure can be made by chemical synthesis methods well known to the ordinarily skilled artisan (see, e.g., Fields et al., Chapter 3 in Synthetic Peptides: A User's Guide, ed. Grant, W. H. Freeman & Co., New York, N.Y., 1992, p. 77). The α-Syn peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of α-Syn peptide immunogen constructs comprising combinatorial library peptides for Th epitopes can be accomplished by providing a mixture of alternative amino acids for coupling at a given variable position.

After complete assembly of the desired α-Syn peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art.

The quality of peptides produced by this chemical process can be controlled and defined and, as a result, reproducibility of α-Syn peptide immunogen constructs, immunogenicity, and yield can be assured. Detailed description of the manufacturing of the α-Syn peptide immunogen construct through solid phase peptide synthesis is shown in Example 1.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs. Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The α-Syn peptide immunogen constructs can also be made using recombinant DNA technology including nucleic acid molecules, vectors, and/or host cells. As such, nucleic acid molecules encoding the α-Syn peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the α-Syn peptide immunogen construct and immunologically functional analogues of the α-Syn G111-D135 fragment derived peptide immunogen constructs. For example, methods can include a step of incubating a host cell containing an expression vector containing a nucleic acid molecule encoding an α-Syn peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the Immunostimulatory complexes comprising α-Syn peptide immunogen constructs and CpG oligodeoxynucleotide (ODN) molecule. Stabilized immunostimulatory complexes (ISC) are derived from a cationic portion of the α-Syn peptide immunogen construct and a polyanionic CpG ODN molecule. The self-assembling system is driven by electrostatic neutralization of charge. Stoichiometry of the molar charge ratio of cationic portion of the α-Syn peptide immunogen construct to anionic oligomer determines extent of association. The non-covalent electrostatic association of α-Syn peptide immunogen construct and CpG ODN is a completely reproducible process. The peptide/CpG ODN immunostimulatory complex aggregates, which facilitate presentation to the “professional” antigen-presenting cells (APC) of the immune system thus further enhancing of the immunogenicity of the complexes. These complexes are easily characterized for quality control during manufacturing. The peptide/CpG ISC are well tolerated in vivo. This novel particulate system comprising CpG ODN and α-Syn G111-D135 fragment derived peptide immunogen constructs was designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen-presenting cells (APC). Consequently, such formulations are novel conceptually and offer potential advantages by promoting the stimulation of immune responses by alternative mechanisms.

c. Methods for the Manufacturing Pharmaceutical Compositions

Various exemplary embodiments also encompass pharmaceutical compositions containing α-Syn peptide immunogen constructs. In certain embodiments, the pharmaceutical compositions employ water in oil emulsions and in suspension with mineral salts.

In order for a pharmaceutical composition to be used by a large population and with prevention of α-Syn aggregation also being part of the goal for administration, safety becomes another important factor for consideration. Despite the use of water-in-oil emulsions in humans for many formulations in clinical trials, Alum remains the major adjuvant for use in formulations due to its safety. Alum or its mineral salts Aluminum phosphate (ADJUPHOS) are, therefore, frequently used as adjuvants in preparation for clinical applications.

d. Methods Using Pharmaceutical Compositions

The present disclosure also includes methods of using pharmaceutical compositions containing α-Syn peptide immunogen constructs.

In certain embodiments, the pharmaceutical compositions containing α-Syn peptide immunogen constructs can be used for:
(a) inhibiting α-Syn aggregation in a host;
(b) inducing disaggregate of preformed α-Syn aggregates in a host;
(c) reducing microglial TNF-alpha and IL6 secretion in a host;
(d) reducing neurodegeneration triggered by exogeneous α-Syn aggregates in a host;
(e) reducing neurodegeneration in α-Syn overexpressing cells;
(f) reducing serum α-Syn levels in a host;
(g) reducing oligomeric α-Syn level in the brain of a host;
(h) reducing neuropathology and recovery of motor activities in a host; and the like.

The above methods comprise administering a pharmaceutical composition comprising a pharmacologically effective amount of an α-Syn peptide immunogen construct to a host in need thereof.

### Specific Embodiments

Specific embodiments of the present invention include, but are not limited to, the following:
(1) An alpha-synuclein (α-Syn) peptide immunogen construct comprising:
   a B cell epitope comprising about 10 to about 25 amino acid residues from a C-terminal fragment of α-Syn corresponding to about amino acid G111 to about amino acid D135 of SEQ ID NO: 1;
   a T helper epitope comprising an amino acid sequence selected from the group consisting of SEQ ID NOs: 70-98; and
   an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 148),
   wherein the B cell epitope is covalently linked to the T helper epitope directly or through the optional heterologous spacer.
(2) The α-Syn peptide immunogen construct of (1), wherein the B cell epitope is selected from the group consisting of SEQ ID NOs: 12-15, 17, and 49-63.
(3) The α-Syn peptide immunogen construct of (1), wherein the T helper epitope is selected from the group consisting of SEQ ID NOs: 81, 83, and 84.
(4) The α-Syn peptide immunogen construct of (1), wherein the optional heterologous spacer is (α, ε-N)Lys or ε-N-Lys-Lys-Lys-Lys (SEQ ID NO: 148).
(5) The α-Syn peptide immunogen construct of (1), wherein the T helper epitope is covalently linked to the amino terminus of the B cell epitope.
(6) The α-Syn peptide immunogen construct of (1), wherein the T helper epitope is covalently linked to the amino terminus of the B cell epitope through the optional heterologous spacer.
(7) The α-Syn peptide immunogen construct of (1) comprising the following formula:

(Th)ₘ-(A)ₙ-(α-Syn C-terminal fragment)-X

or

(α-Syn C-terminal fragment)-(A)ₙ-(Th)ₘ-X
wherein
Th is the T helper epitope;
A is the heterologous spacer;
(α-Syn C-terminal fragment) is the B cell epitope;
X is an α-COOH or α-CONH₂ of an amino acid;
m is from 1 to about 4; and
n is from 1 to about 10.
(8) The α-Syn peptide immunogen construct of (1), comprising the amino acid sequence selected from the group consisting of SEQ ID NOs: 107, 108, 111-113, and 115-147.
(9) The α-Syn peptide immunogen construct of (1), comprising the amino acid sequence selected from the group consisting of SEQ ID NOs: 107, 108, and 111-113.
(10) A composition comprising the α-Syn peptide immunogen construct of (1).
(11) A composition comprising more than one α-Syn peptide immunogen construct of (1).
(12) The composition of (11), wherein the α-Syn peptide immunogen constructs have amino acid sequences of SEQ ID NOs: 112 and 113.
(13) A pharmaceutical composition comprising the α-Syn peptide immunogen construct of (1) and a pharmaceutically acceptable delivery vehicle and/or adjuvant.
(14) The pharmaceutical composition of (13), wherein
   a. the α-Syn peptide immunogen construct is selected from the group consisting of SEQ ID NOs: 107, 108, 111-113, and 115-147; and
   b. the adjuvant is a mineral salt of aluminum selected from the group consisting of Al(OH)₃ or AlPO₄.
(15) The pharmaceutical composition of (13), wherein
   a. the α-Syn peptide immunogen construct is selected from the group consisting of SEQ ID NOs: 107, 108, 111-113, and 115-147; and
   b. the α-Syn peptide immunogen construct is mixed with an CpG oligodeoxynucleotide (ODN) to form a stabilized immunostimulatory complex.
(16) An isolated antibody or epitope-binding fragment thereof that specifically binds to the B cell epitope of the α-Syn peptide immunogen construct of (1).
(17) The isolated antibody or epitope-binding fragment thereof according to (16) bound to the α-Syn peptide immunogen construct.
(18) An isolated antibody or epitope-biding fragment thereof that specifically binds to the B cell epitope of the α-Syn peptide immunogen construct of (9).
(19) A composition comprising the isolated antibody or epitope-binding fragment thereof according to (16).
(20) A composition comprising the isolated antibody or epitope-binding fragment thereof according to (18).
(21) The composition of (20), comprising a mixture of
   a. an isolated antibody or epitope-binding fragment thereof that specifically binds to the B cell epitope of SEQ ID NO: 112; and
   b. an isolated antibody or epitope-binding fragment thereof that specifically binds to the B cell epitope of SEQ ID NO: 113.
(22) A method of producing antibodies that recognize α-Syn in a host comprising administering to the host a composition comprising the α-Syn peptide immunogen of (1) and a delivery vehicle and/or adjuvant.
(23) A method of inhibiting α-Syn aggregation in an animal comprising administering a pharmacologically effective amount of the α-Syn peptide immunogen of (1) to the animal.
(24) A method of reducing the amount of α-Syn aggregates in an animal comprising administering a pharmacologically effective amount of the α-Syn peptide immunogen of (1) to the animal.
(25) A method of identifying α-Syn aggregates of different sizes in a biological sample comprising:
   a. exposing the biological sample to the antibody or epitope-binding fragment thereof according to (16) under conditions that allow the antibody or epitope-binding fragment thereof to bind to the α-Syn aggregates; and
   b. detecting the amount of the antibody or epitope-binding fragment thereof bound to the α-Syn aggregates in the biological sample.

A detailed description of the procedures used is provided in the following Examples.

### Example 1

### Synthesis of Alpha Synuclein Related Peptides and Preparation of Formulations Thereof

a. Synthesis of α-Syn C-Terminal Fragments

Methods for synthesizing designer α-Syn C-terminal fragments that were included in the development effort of α-Syn peptide immunogen constructs are described. The peptides were synthesized in small-scale amounts that are useful for serological assays, laboratory pilot and field studies, as well as large-scale (kilogram) amounts, which are useful for industrial/commercial production of pharmaceutical compositions. A large repertoire of α-Syn related antigenic peptides having sequences with lengths from approximately 10 to 40 amino acids were designed for the screening and selection of the most optimal peptide constructs for use in an efficacious α-Syn peptide immunogen construct.

Representative full length α-Syn (SEQ ID NO:1) and β-Syn (SEQ ID No: 2), α-Syn segments such as α-Syn₁₁₁₋₁₃₂, α-Sy₁₂₆₋₁₃₅, 10-mer peptides etc. employed for epitope mapping in various serological assays are identified in Table 1 (SEQ ID NOs: 1 and 3 to 69). Selected α-Syn fragments were made into α-Syn peptide immunogen constructs by synthetically linking to a carefully designed helper T cell (Th) epitope derived from pathogen proteins including Measles Virus Fusion protein (MVF), Hepatitis B Surface Antigen protein (HBsAg) influenza, Clostridum tetani, and Epstein-Barr virus (EBV) identified in Table 2 (SEQ ID NOs: 70-98). The Th epitopes were used either in a single sequence (SEQ ID NOs: 70-78 and 83-98) or a combinatorial library (SEQ ID NOs: 79-82) to enhance the immunogenicity of their respective α-Syn peptide immunogen constructs.

Representative α-Syn peptide immunogen constructs selected from over 100 peptide constructs are identified in Table 3 (SEQ ID NOs: 99-147). All peptides used for immunogenicity studies or related serological tests for detection and/or measurement of anti-α-Syn antibodies were synthesized on a small scale using F-moc chemistry by peptide synthesizers of Applied BioSystems Models 430A, 431 and/or 433. Each peptide was produced by an independent synthesis on a solid-phase support, with F-moc protection at the N-terminus and side chain protecting groups of trifunctional amino acids. Completed peptides were cleaved from the solid support and side chain protecting groups were removed by 90% Trifluoroacetic acid (TFA). Synthetic peptide preparations were evaluated by Matrix-Assisted Laser Desorption/Ionization-Time-Of-Flight (MALDI-TOF) Mass Spectrometry to ensure correct amino acid content. Each synthetic peptide was also evaluated by Reverse Phase HPLC (RP-HPLC) to confirm the synthesis profile and concentration of the preparation. Despite rigorous control of the synthesis process (including stepwise monitoring the coupling efficiency), peptide analogues were also produced due to unintended events during elongation cycles, including amino acid insertion, deletion, substitution, and premature termination. Thus, synthesized preparations typically included multiple peptide analogues along with the targeted peptide. Despite the inclusion of such unintended peptide analogues, the resulting synthesized peptide preparations were nevertheless suitable for use in immunological applications including immunodiagnosis (as antibody capture antigens) and pharmaceutical compositions (as peptide immunogens). Typically, such peptide analogues, either intentionally designed or generated through synthetic process as a mixture of byproducts, are frequently as effective as a purified preparation of the desired peptide, as long as a discerning QC procedure is developed to monitor both the manufacturing process and the product evaluation process to guarantee the reproducibility and efficacy of the final product employing these peptides. Large scale peptide syntheses in the multi-hundred to kilo gram quantities were conducted on a customized automated peptide synthesizer UBI2003 or the like at 15 mmole to 50 mmole scale. For active ingredients used in the final pharmaceutical composition for clinical trials, α-Syn peptide constructs were purified by preparative RP-HPLC under a shallow elution gradient and characterized by MALDI-TOF mass spectrometry, amino acid analysis and RP-HPLC for purity and identity.

b. Preparation of Compositions Containing α-Syn Peptide Immunogen Constructs

Formulations employing water in oil emulsions and in suspension with mineral salts were prepared. In order for a pharmaceutical composition designed to be used by a large population and with prevention also being part of the goal for administration, safety becomes another important factor for consideration. Despite the use of water-in-oil emulsions in humans for many pharmaceutical compositions in clinical trials, Alum remains the major adjuvant for use in pharmaceutical composition due to its safety. Alum or its mineral salts ADJUPHOS (Aluminum phosphate) are therefore frequently used as adjuvants in preparation for clinical applications.

Briefly, the formulations specified in each of the study groups described below generally contained all types of designer the α-Syn peptide immunogen constructs. Over 100 designer α-Syn peptide immunogen constructs were initially evaluated in guinea pigs for their relative immunogenicity with the corresponding α-Syn peptide representative of the immunogen's B epitope peptide and also for assessment of serological cross-reactivities amongst the varying homologous peptides by ELISA assays with plates coated with different peptides selected from those with SEQ ID NOs: 1-153.

The α-Syn peptide immunogen constructs were prepared (i) in a water-in-oil emulsion with Seppic Montanide™ ISA 51 as the approved oil for human use, or (ii) mixed with mineral salts ADJUPHOS (Aluminum phosphate) or ALHYDROGEL (Alum), at varying amounts of peptide constructs, as specified. Compositions were typically prepared by dissolving the α-Syn peptide immunogen constructs in water at about 20 to 800 μg/mL and formulated with Montanide™ ISA 51 into water-in-oil emulsions (1:1 in volume) or with mineral salts or ALHYDROGEL (Alum) (1:1 in volume). The compositions were kept at room temperature for about 30 min and mixed by vortex for about 10 to 15 seconds prior to immunization. Some animals were immunized with 2 to 3 doses of a specific composition, which were administered at time 0 (prime) and 3 week post initial immunization (wpi) (booster), optionally 5 or 6 wpi for a second boost, by intramuscular route. These immunized animals were then tested with selected B epitope peptide(s) to evaluate the immunogenicity of the various α-Syn peptide immunogen constructs present in the formulation as well as their cross-reactivity with related target peptides or proteins. Those α-Syn peptide immunogen constructs with potent immunogenicity in the initial screening in guinea pigs were then further tested in both water-in-oil emulsion, mineral salts, and alum-based formulations in primates for dosing regimens over a specified period as dictated by the immunizations protocols.

Only the most promising α-Syn peptide immunogen constructs were further assessed extensively prior to being incorporated into final formulations for immunogenicity, duration, toxicity and efficacy studies in GLP guided preclinical studies in preparation for submission of an Investigational New Drug application and clinical trials in patients with synucleinopathies.

### Example 2

### Preparation of Recombinant Alpha Synuclein Protein

Cloning of α-Syn gene into pGEX-4T1 vector was previously described in Neurotoxicology and teratology 2004, 26 (3): 397-406. The target sequence (SEQ ID NOs: 1) was inserted into pGEX-4T1 vector between BamHI and XhoI restriction sites. The fragment was generated by polymerase chain reaction (PCR) using KAPA HiFi DNA polymerase (Kapa Biosystems, Inc., Woburn, Mass., USA). Primer sequences are as follows: forward primer, 5′-cgggatccgatgtgtttatgaaaggtctgag-3′ (SEQ ID NO: 149); reverse primer, 5′-ggaattccgatgtgtttatgaaaggtctgag-3′ (SEQ ID NO: 150). The PCR condition was as follows: denaturation at 94° C. for 1 min followed by 30 cycles of denaturation at 94° C. for 15 s, annealing at 60° C. for 30 s and extension at 68° C. for 2 min, and terminated after additional 5 min at 68° C. Site-directed mutagenesis of A53T α-Syn was performed using the Q5 Site-Directed Mutagenesis Kit (New England BioLabs, Beverly, Mass., USA). Primer sequences for mutant α-Syn are as follows: forward primer, 5′-tcatggtgtgaccaccgttgcag-3′ (SEQ ID NO: 151); reverse primer, 5′-accacgccttattggttttg-3′ (SEQ ID NO: 152).

The α-Syn cloned into pGEX-4T1 GST vector was transformed to E. coli BL21 (DE3) for protein expression. E. coli was cultured in the LB broth at 37° C. and Isopropyl β-D-1-thiogalactopyranoside (IPTG) was added to a final concentration of 4 mM when OD600 reached 0.8. After 4 hr incubation, the cells were collected by centrifugation at 5,000×g for 20 min at 4° C. The collected cells were resuspended in PBS, disrupted by sonication on ice and then centrifuged at 5,000×g for 20 min. The supernatant fraction was loaded onto a Glutathione Sepharose-4B column (GE Healthcare) equilibrated with PBS. After three times washing with PBS, 1 mL thrombin (20 U/mL in PBS) was added for overnight digestion at 4° C. to release GST from the fusion protein. Tag-free α-Syn were then eluted, with the thrombin subsequently removed by HiTrap Benzamidine FF column (GE Healthcare). The dialyzed α-Syn was frozen immediately at −80° C. Purified α-Syn with 14 kDa MW were identified by western blotting with anti-α-Syn antibody (1:2000, Millipore, targeting α-Syn₁₁₁₋₁₃₁) after separation by 10% SDS-PAGE.

### Example 3

### Serological Assays and Reagents

Serological assays and reagents for evaluating functional immunogenicity of the synthetic peptide constructs and formulations thereof are described in details below.

a. Peptide-Based ELISA Tests for Antibody Specificity Analysis

ELISA assays for evaluating immune serum samples described in the following Examples were developed and described below. The wells of 96-well plates were coated individually for 1 hour at 37° C. with 100 μL of target peptide α-Syn fragments A85-A140, A91-A140, A101-A140, A111-A140, D121-A140, E126-A140, K97-D135, G101-D135, G111-D135, D121-D135, E123-D135, E126-D135, G101-132, and G111-G132 peptide (SEQ ID NOs: 4-17), at 2 μg/mL (unless noted otherwise), in 10 mM NaHCO₃ buffer, pH 9.5 (unless noted otherwise).

b. Assessment of Antibody Reactivity Towards Th Peptide by Th Peptide Based ELISA Tests

The peptide (SEQ ID Nos: 70-98)-coated wells were incubated with 250 μL of 3% by weight of gelatin in PBS in 37° C. for 1 hour to block non-specific protein binding sites, followed by three washes with PBS containing 0.05% by volume of TWEEN® 20 and dried. Sera to be analyzed were diluted 1:20 (unless noted otherwise) with PBS containing 20% by volume normal goat serum, 1% by weight gelatin and 0.05% by volume TWEEN® 20. One hundred microliters (100 μL) of the diluted specimens (e.g., serum, plasma) were added to each of the wells and allowed to react for 60 minutes at 37° C. The wells were then washed six times with 0.05% by volume TWEEN® 20 in PBS in order to remove unbound antibodies. Horseradish peroxidase (HRP)-conjugated species (e.g., mouse, guinea pig, or human) specific goat anti-IgG, was used as a labeled tracer to bind with the antibody/peptide antigen complex formed in positive wells. One hundred microliters of the peroxidase-labeled goat anti-IgG, at a pre-titered optimal dilution and in 1% by volume normal goat serum with 0.05% by volume TWEEN® 20 in PBS, was added to each well and incubated at 37° C. for another 30 minutes. The wells were washed six times with 0.05% by volume TWEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0M H₂SO₄ and absorbance at 450 nm (A₄₅₀) determined. For the determination of antibody titers of the immunized animals that received the various α-Syn derived peptide immunogens, 10-fold serial dilutions of sera from 1:100 to 1:10,000 were tested, and the titer of a tested serum, expressed as Log₁₀, was calculated by linear regression analysis of the A₄₅₀ with the cutoff A₄₅₀ set at 0.5.

c. Fine Specificity Analysis and Epitope Mapping to α-Syn Fragments by B Cell Epitope Cluster 10-Mer Peptide-Based ELISA Tests

Fine specificity analyses of anti-α-Syn antibodies in immunized hosts were determined by epitope mapping. Briefly, the wells of 96-well plates were coated with individual α-Syn 10-mer peptides (SEQ ID NOs: 18 to 69) at 0.5 μg per 0.1 mL per well and then 100 μL serum samples (1:100 dilution in PBS) were incubated in 10-mer plate wells in duplicate following the steps of the antibody ELISA method described above. The B cell epitope of the α-Syn peptide immunogen construct and related fine specificity analyses of immune sera's anti-α-Syn antibodies in immunized hosts were tested also with corresponding α-Syn peptides (SEQ ID No:99, 102, 108, 110, 112, 113) or its fragment without the spacer and Th sequences, or with β-Syn (SEQ ID NO: 153) for additional reactivity and specificity confirmation.

d. Immunogenicity Evaluation

Preimmune and immune serum samples from animals were collected according to experimental immunization protocols and heated at 56° C. for 30 minutes to inactivate serum complement factors. Following the administration of the pharmaceutical composition, blood samples were obtained according to protocols and their immunogenicity against specific target site(s) evaluated. Serially diluted sera were tested and positive titers were expressed as Log₁₀ of the reciprocal dilution. Immunogenicity of a particular pharmaceutical composition is assessed by its ability to elicit high titer B cell antibody response directed against the desired epitope specificity within the target antigen while maintaining a low to negligible antibody reactivity towards the “Helper T cell epitopes” employed to provide enhancement of the desired B cell responses.

e. Immunoassay for α-Syn Level in Mouse Immune Sera

Serum α-Syn levels in mice receiving α-Syn derived peptide immunogens were measured by a sandwich ELISA (Cloud-clon, SEB222Mu) using anti-α-Syn antibodies as capture antibody and biotin-labeled anti-α-Syn antibody as detection antibody. Briefly, the antibody was immobilized on 96-well plates at 100 ng/well in coating buffer (15 mM Na₂CO₃, 35 mM NaHCO₃, pH 9.6) and incubated at 4° C. overnight. Coated wells were blocked by 200 μL/well of assay diluents (0.5% BSA, 0.05% TWEEN®-20, 0.02% ProClin 300 in PBS) at room temperature for 1 hour. Plates were washed 3 times with 200 μL/well of wash buffer (PBS with 0.05% TWEEN®-20). Purified recombinant α-Syn was used to generate a standard curve (range 156 to 1250 ng/mL by 2-fold serial dilution) in assay diluent with 5% mouse sera. 50 μL of the diluted sera (1:20) and standards were added to coated wells. The incubation was carried out at room temperature for 1 hour. All wells were aspirated and washed 6 times with 200 μL/well of wash buffer. The captured human α-Syn was incubated with 100 μL of detection antibody solution (50 ng/ml of biotin labeled HP6029 in assay diluent) at room temperature for 1 hour. Then, the bound biotin-HP6029 was detected using streptavidin poly-HRP (1:10,000 dilution, Thermo Pierce) for 1 hour (100 μL/well). All wells were aspirated and washed 6 times with 200 μL/well of wash buffer and the reaction was stopped by addition of 100 μL/well of 1M H₂SO₄. The standard curve was created by using the SoftMax Pro software (Molecular Devices) to generate a four parameter logistic curve-fit and used to calculate the concentrations of α-Syn in all tested samples. Student t tests were used to compare data by using the Prism software.

f. Preparation of α-Syn Aggregates with Recombinant α-Syn

To prepare aggregated α-Syn, the purified wile-type or A53T-mutated α-Syn [0.1 μg/μL in 100 μL, PBS/KCl aggregation buffer (2.5 mM MgCl₂, 50 mM HEPES and 150 mM KCl in 1×PBS, pH 7.4)] was incubated at 37° C. in 1.5 mL Eppendorf tubes for 7 days in a Thermomixer (Eppendorf) without shaking. Aggregated α-Syn was immediately frozen at −80° C. for later use.

g. Purification of Anti-α-Syn Antibodies

Anti-α-Syn Antibodies were purified from sera collected at 3 to 15 weeks post-injection (WPI) of guinea pigs immunized with α-Syn peptide immunogen constructs containing peptides of different sequences (SEQ ID NOs: 99-121) by using an affinity column (Thermo Scientific, Rockford). Briefly, after buffer (0.1 M phosphate and 0.15 M sodium chloride, pH 7.2) equilibration, 400 μL of serum was added into the Nab Protein G Spin column followed by end-over-end mixing for 10 min and centrifugation at 5,800×g for 1 min. The column was washed with binding buffer (400 μL) for three times. Subsequently, elution buffer (400 μL, 0.1 M glycine pH 2.0) was added into the spin column to elute the antibodies after centrifuging at 5,800×g for 1 min. The eluted antibodies were mixed with neutralization buffer (400 μL, 0.1 M Tris pH 8.0) and the concentrations of these purified antibodies were measured by using Nan-Drop at OD280, with BSA (bovine serum albumin) as the standard.

h. Specificity of Anti-α-Syn Antibodies Purified from Guinea Pig Antisera Immunized with Different α-Syn Peptide Immunogen Constructs of Different Sizes

Western blot was used to screen anti-α-Syn antibodies purified from guinea pig antisera immunized with different α-Syn peptide immunogen constructs for the binding specificity to α-Syn molecular complex of different sizes. 20 μM of α-Syn were separated on 12% Tris-glycine SDS-PAGE and transferred to nitrocellulose (NC) membrane before photo-induced cross-linking (PICUP) treatment. The membrane was incubated with anti-α-Syn antibodies purified from guinea pigs antisera at 1 μg/mL, and then incubated with donkey anti-guinea pig antibody conjugated HRP (706-035-148, Jackson). The blot was visualized with chemiluminescence reagent Western Lightning ECL Pro (PerkinElmer). As the result, the monomeric α-Syn (Mw 14,460 Da) was blotted around the size of 14 kDa, while dimer, trimer, or oligomers had their molecular weights several folds greater than the monomeric α-Syn size of 14 kDa. The commercial antibody which is able to detect various oligomeric species such as dimers, trimers, and larger oligomers, Syn211 (Abcam), was employed as a positive control.

i. Dot Blot Assay with Different Species of Amyloidogenic Proteins

Preparation of α-helix monomers, β-sheet monomers, β-sheet oligomers, and β-sheet fibrils of Aβ1-42, Tau, and α-Syn are described as follows.
1. Aβ₁₋₄₂ α-helix monomers: 20 μg of Aβ₁₋₄₂ β-sheet monomers (50 μL) was added in 1×PBS containing with 20% trifluoroacetic acid and 20% hexafluoroisopropanol (10 μL) and incubated at 4° C. for 24 hrs to form the α-helix monomers.
2. Aβ₁₋₄₂ β-sheet monomers: 60 μg of Aβ₁₋₄₂ in 120 μL 1×PBS containing 5% TFA aggregated at 37° C. for 24 hrs was transferred onto a 10 kDa cut-off filter (Millipore) to recover the β-sheet monomers.
3. Aβ₁₋₄₂ β-sheet oligomers: 60 μg of Aβ₁₋₄₂ in 120 μL 1×PBS aggregated at 37° C. for 3 days was sonicated on ice and transferred onto 10 and 30 kDa cut-off filters (Millipore) to recover the β-sheet oligomeric fibrils of less than 35 kDa.
4. Aβ₁₋₄₂ β-sheet fibrils: 60 μg of Aβ₁₋₄₂ in 120 μL 1×PBS aggregated at 37° C. for 3 days was sonicated on ice and transferred onto 30 kDa cut-off filters (Millipore) to isolate the β-sheet fibrils.
5. α-Syn α-helix monomers: 40 μg of freshly prepared α-Syn was dissolved in cold 100 μL 1×PBS at 4° C. and immediately transferred onto a 10 kDa cut-off filter (Millipore) to recover the α-helix monomer.
6. α-Syn β-sheet monomers: 40 μg of α-Syn incubated in 100 μL PBS/KCl buffer at 37° C. for 24 hrs was transferred onto a 10 kDa cut-off filter (Millpore) to recover the β-sheet monomers.
7. α-Syn β-sheet oligomers: 40 μg of α-Syn aggregated in 100 μL PBS/KCl buffer at 37° C. for 8 days was sonicated on ice and then transferred onto 30 and 100 kDa cut-off filters to recover the β-sheet oligomers.
8. α-Syn β-sheet fibrils: 40 μg of α-Syn aggregated in 100 μL PBS/KCl buffer at 37° C. for 8 days was sonicated on ice and then transferred onto 30 and 100 kDa cut-off filters to isolate the β-sheet fibrils. Tau441 α-helix monomers: 60 μg of Tau prepared in 100 μL 1×PBS at 4° C. was transferred onto a 100 kDa cut-off to recover the α-helix monomers.
9. Tau441 β-sheet monomers: 60 μg of Tau aggregated in 100 μL 1×PBS with 10 unit/mL heparin at 25° C. for 48 hrs was transferred onto a 100 kDa cut-off filter at 4° C. to recover the β-sheet monomers.
10. Tau441 β-sheet oligomers: 60 μg of Tau aggregated in 100 μL 1×PBS with 10 unit/mL heparin at 37° C. for 48 hrs was transferred onto 100 and 300 kDa cut-off filters (Pall) at 4° C. to recover the β-sheet oligomers.
11. Tau441 β-sheet fibrils: 60 μg of Tau aggregated in 100 μL 1×PBS with 10 unit/mL heparin at 37° C. for 6 days was transferred onto 300 kDa cut-off filters (Pall) at 4° C. to isolate the β-sheet fibrils.

These monomers and oligomers were verified by Thioflavin-T (ThT, Sigma) fluorescence or PAGE (polyacrylamide gel electrophoresis). The concentrations of the amyloidogenic proteins were measured by Nano-Drop with commercial amyloidogenic Aβ₁₋₄₂ stock as the standard. These monomers and oligomers were spotted individually onto PVDF membranes with the amount of 3 μg for Aβ₁₋₄₂, 4 μg for α-Syn, and 7 μg for Tau. The membranes were incubated with the anti-α-Syn antibodies purified from guinea pigs antisera (1:1000 dilution) as primary antibody, followed by hybridization with the anti-guinea pig HRP-conjugated secondary antibody (1:5000; Vector Laboratories). The membranes were treated with Luminata Western HRP Substrates (Bio-Rad, Hercules, Calif., USA) and the signals were detected with a ChemiDoc-It 810 digital image system (UVP Inc., Upland, Calif., USA).

j. Binding Specificity to Aggregated α-Syn in α-Syn-Overexpressing PC12 Cells Upon Nerve Growth Factor (NGF) Treatment

Immunocytochemistry (ICC) with anti-α-Syn antibodies purified from guinea pigs antisera collected at 8 or 9 WPI on parental-PC12, mock-controlled PC12 and α-Syn-overexpressing PC12 cells after NGF treatment were performed to evaluate the binding affinity of the antibodies elicited after immunization. The cell nuclei were counterstained with DAPI (4′,6-diamidino-2-phenylindole). Photographs were taken with a fluorescence microscope, and the ratio of the number of positively stained cells against the total number of cells were categorically scored with −, +, ++ and +++, representing <1%, 1-15%, 16-50%, >50%.

### Example 4

### Cells and Animals Used in Immunogenicity and Efficacy Studies

a. α-Syn-Overexpressing PC12 Cells:

The pZD/X0L-L-α-Syn plasmid was constructed by inserting the cDNA sequence encoding full-length human wild-type α-Syn or A53T mutated α-Syn into the pZD/XOL-L vector with CMV promotor. The constructs were transfected into PC12 cells using Lipofectamine LTX transfection reagent (Invitrogen, Carlsbad, Calif., USA) according to manufacturer's procedure. 2.5 μL of the transfection mixture, 500 μL of Opti-MEM medium 2.5 μL PLUS reagent, and 8.75 μL lipofectamine LTX were mixed and then incubated for 25 mins at room temperature. After replacing the culture medium with 1.5 mL of RMPI 1640 growth medium, 500 μL of the transfection mixture was added directly to each well followed by incubations at 37° C. for one day. The transfection efficiency was confirmed with PCR and western blotting.

b. Guinea Pigs:

Immunogenicity studies were conducted in mature, naïve, adult male and female Duncan-Hartley guinea pigs (300-350 g/BW). The experiments utilized at least 3 Guinea pigs per group. Protocols involving Duncan-Hartley guinea pigs (8-12 weeks of age; Covance Research Laboratories, Denver, Pa., USA), were performed under approved IACUC applications at the contracted animal facility as well as at UBI, as sponsor.

c. Fibrillar α-Syn-Inoculated Parkinson Mice Model:

FVB female mice (weight ranging 25-30 g) were maintained on a 12-hr light: 12-hr dark cycle, and animal care was in accordance with AAALAC approved guidelines. Fibrillar α-Syn was prepared by incubating α-Syn peptides (5 mg/mL) at 37° C. in 0.1% NaN₃-containing PBS/high KCl buffer without shaking for 7 days. Fibrillization was monitored by measuring ThT fluorescence and the confirmation was made when the signal increased more than 3-fold of the original α-Syn monomer. Western blotting was also used to validate the aggregation of α-Syn prior to the inoculation into unilateral substantia nigra (anterior-posterior; −3.0 mm; medial-lateral: −1.3 mm; dorsal-ventral: −4.7 mm from the bregma and dura) and dorsal neostriatum (anterior-posterior; +0.2 mm; medial-lateral: −2 mm; dorsal-ventral: −3.2 mm from the bregma and dura) of the isoflurane anesthetized animals.

d. MPP+ Induced Parkinson Mice Model:

Balb/c female mice (weight ranging 18-20 g) were maintained on a 12-hr light: 12-hr dark cycle, and animal care was in accordance with AAALAC approved guidelines. MPP+ iodide (Sigma, St. Luis, Mo.) was dissolved in saline and injected with 10 μl of solution containing 18 μg of MPP+ iodide (0.8 mg/kg) into the unilateral ventricle of the anesthetized animal. The stereotaxic coordinates of injection site were: bregma −1.0 mm, lateral 1.0 mm, depth 2.0 mm.

### Example 5

### Design Rationale, Screening, Identification and Optimization of Multi-Component Pharmaceutical Compositions Incorporating Alpha Synuclein Peptide Immunogen Constructs

a. Design History

Each α-Syn peptide immunogen construct or immunotherapeutic product requires its own design focus and approach based on the specific disease mechanism and the target protein(s) required for intervention. The targets that designs are modeled after can include cellular proteins involved in a disease pathway or an infectious agent in which several proteins from the pathogen may be involved. The process from research to commercialization is very long typically requires one or more decades to accomplish.

An extensive process of serological validation is required once the target molecule is selected. Identification and distribution of the B cell and T cell epitopes within the target molecule is important to the molecular α-Syn peptide immunogen construct design. Once the target B cell epitope is recognized, consecutive pilot immunogenicity studies in small animals are conducted to evaluate the functional properties of the antibodies elicited by the pharmaceutical compositions of the designer peptides. Such serological application is then carried out in animals of the target species for further validation of the α-Syn peptide immunogen construct immunogenicity and functional properties of the elicited antibodies. All studies are conducted in multiple parallel groups with sera collected from the immunized hosts for evaluation. Early immunogenicity studies in the target species or in non-human primate in the case of human pharmaceutical compositions, are also carried out to further validate the immunogenicity and direction of the design. Target peptides are then prepared in varying mixtures to evaluate subtle difference in functional property related to the respective interactions among peptide constructs when used in combinations to prepare for respective formulation designs. After additional evaluations, the final peptide constructs, peptide compositions and formulations thereof, along with the respective physical parameters of the formulations are established leading to the final product development process.

b. Design and Validation of α-Syn Derived Peptide Immunogen Constructs for Pharmaceutical Compositions with Potential to Treat Patients with Synucleinopathies

In order to generate the most potent peptide constructs for incorporation into the pharmaceutical compositions, a large repertoire of promiscuous T helper epitopes derived from various pathogens or artificially T helper epitopes further designed from Measles Virus Fusion (MVF) protein sequence or Hepatitis B Surface Antigen (HBsAg) protein were made into immunogenicity studies in guinea pigs. A representative study of α-Syn₁₂₆₋₁₄₀, α-Syn₁₂₁₋₁₄₀, α-Syn₁₁₁₋₁₄₀, α-Syn₁₀₁₋₁₄₀, α-Syn₉₁₋₁₄₀, α-Syn₈₅₋₁₄₀, α-Syn₁₂₁₋₁₃₅, α-Syn₁₁₁₋₁₃₅, α-Syn₁₀₁₋₁₃₅, α-Syn₉₇₋₁₃₅, α-Syn₁₂₃₋₁₃₅, α-Syn₁₂₆₋₁₃₅, α-Syn₁₁₁₋₁₃₂, and α-Syn₁₀₁₋₁₃₂ derived peptide constructs as shown in Table 3 (SEQ ID NOs: 99 to 121) where α-Syn peptide was linked through εK and/or KKK as spacer(s) with individual promiscuous T helper epitopes.

i) Selection of C-Terminal Part of α-Syn as Target for Peptide Immunogen Design.

α-Syn is an intrinsically disordered protein. It consists of 140 amino acids and is divided into three regions. The N-terminal region (residues 1-60) is capable of forming an amphipathic helix which is a typical conformation for membrane recognition and association. The central region containing residues 61-95 is well known as the non-amyloid β component (NAC) firstly identified in AD senile plaques. This region features a high propensity to form a β-rich conformation and is highly aggregation-prone. Different types of post-translational modifications within this region show distinct effects on modulating α-Syn aggregation. The C-terminal region with residues 96-140 is rich of proline and negatively charged residues which is a common characteristic found in intrinsically disordered proteins to maintaining solubility. This C-terminal domain is present in a random coil structure due to its low hydrophobicity and high net negative charge. In vitro studies have revealed that α-Syn aggregation can be induced by reduction of pH which neutralizes these negative charges. α-Syn features an extreme conformational diversity, which adapts to different conditions in the states of membrane binding, cytosol, and amyloid aggregation and fulfills versatile functions. Upon much consideration, the C-terminal random coil and intrinsically disordered region, important for the protein to maintain solubility, was selected as the target for peptide immunogen design as this region would be most susceptible to modulation by antibody or other physical factors than the N-terminal amphipathic helix and the central β-rich conformation regions.

ii) Identification of Autologous Th Epitopes for Exclusion in α-Syn B Epitope Design.

Preliminary immunogenicity analysis confirmed the presence of helper T cell epitope(s) structure feature in the C-terminus of α-Syn where deletion of peptide sequence from N-terminus of the α-Syn sequence rendered α-Syn₁₂₆₋₁₄₀ (SEQ ID NO: 9), α-Syn₁₂₁₋₁₄₀ (SEQ ID NO: 8), α-Syn₁₁₁₋₁₄₀ (SEQ ID NO: 7) peptides totally non-immunogenic whereas some modest immunogenicity was observed with α-Syn₁₀₁₋₁₄₀ (SEQ ID NO: 6), α-Syn₉₁₋₁₄₀ (SEQ ID NO: 5), and α-Syn₈₅₋₁₄₀ (SEQ ID NO: 4) peptides (Table 4) indicative of presence of potential autologous Th like structure within the C-terminal sequence. Inclusion of such sequence in the B epitope(s) design could potentially cause brain inflammation upon booster immunization due to activation of autologous T cells, as in the previous of AN1792 for Alzheimer's disease vaccine. This finding therefore requires us to design α-Syn peptide immunogen constructs with B cell epitope(s) beginning at Amino Acid residue G111 so as to avoid any chance of including autologous T cell epitope(s) in the B epitope design.

iii) Ranking of the Heterologous T Helper Epitopes and their Inclusion in the α-Syn Peptide Immunogen Constructs Design to Restore and Enhance the Immunogenicity of the Selected α-Syn B Epitope Peptide.

Table 2 lists a total of 29 heterologous Th epitopes (SEQ ID NOs: 70-98) which had been tested within our group for their relative potency in multispecies, from mice, rats, guinea pigs, baboons, macaques etc., to enhance B cell epitope immunogenicity. As shown in Table 5, UBITh1 (SEQ ID NO: 83) and UBITh2 (SEQ ID NO: 84) T cell epitopes derived from MvF protein can both potentiate the nonimmunogenic α-Syn101-140 (SEQ ID NO: 6) peptide to strong and moderate immunogenicity respectively. Extensive testing of multiple α-Syn derived peptide immunogen constructs had been executed to allow ranking of relative immunogenicity amongst these immunogen constructs. Similar immunopotentiating activity is found with UBITh3 (SEQ ID NO: 81) when it is covalently linked through a spacer to various C-terminal α-Syn peptides (SEQ ID NOs: 4 to 9) as illustrated in Table 6 when tested in an ELISA with plate coated with a long α-Syn peptide A91-A140 (SEQ ID NO: 5).

iv) Assessment of Immunogenicity of C-Terminal α-Syn Peptide Immunogen Constructs for their Antibody Reactivities with Corresponding α-Syn and β-Syn.

The synuclein family includes three known proteins: α-Syn, β-Syn, and gamma-synuclein. All synucleins have in common a highly conserved alpha-helical lipid-binding motif with similarity to the class-A2 lipid-binding domains of the exchangeable apolipoproteins. β-Syn is highly homologous to α-Syn. β-Syn is suggested to be an inhibitor of α-Syn aggregation, which occurs in neurodegenerative diseases such as Parkinson's disease. Thus, β-Syn may protect the central nervous system from the neurotoxic effects of α-Syn. It is therefore preferable to have the α-Syn peptide immunogen constructs to elicit antibodies that preferentially react with α-Synuclein and not the corresponding aggregation protective β-Syn. When testing the six peptide immunogen constructs all with C-terminus ending with A140, all of the antibodies derived from the immune sera of these constructs showed significant crossreactivity with the corresponding size β-Syn as shown in Table 6. Upon a close scrutiny of the sequence homology between α-Syn and β-Syn (SEQ ID NOs:1 and 2), the sequence corresponding to the C-terminus five amino acids YEPEA were shown to be identical between the two proteins. It is, therefore, desirable to design B epitope(s) excluding the sequence containing these YEPEA five amino acids. The finding from immunogenicity studies shown in Table 6 thus led to deletion of YEPEA (Y136 to A140) in our B epitope(s) design. Upon incorporation of spacer sequence and, for example, the artificial T-helper peptide UBITh1 (SEQ ID NO:83) into the α-Syn peptide immunogen construct design employing B cell epitope sequences excluding YEPEA tail as shown by the α-Syn peptide immunogens (SEQ ID NOs: 107-114) in Table 7, all became highly immunogenic when assessed on a long α-Syn peptide K97-A140 (SEQ ID NO:110). None of the immune sera reacted with β-Syn. Taken data obtained from Tables 6 and 7, the B epitope design for peptide immunogen constructs would therefore be limited to α-Syn G111 to D135 and fragments thereof.

v) Antibodies Elicited by αSyn Peptide Immunogen Constructs Reacted Exclusively with Beta-Sheet Monomer, Oligomer or Fibril but not the α-Helix Monomer.

Although we had employed sound rationales in our design of α-Syn peptide immunogens, it was surprising to find that the antibodies generated from the designed α-Syn peptide immunogen constructs with B epitopes having their sequences beginning at G111 and ending at D135 or fragments thereof, the elicited antibodies are reactive specifically with β-sheet α-Syn monomer, oligomer, and fibril; and not reactive with β-sheet Aβ₁₋₄₂ or Tau1-441 therefore offering the ideal α-Syn peptide immunogen construct candidates as shown representatively by α-Syn peptide immunogen constructs (SEQ ID Nos: 112 and 113) in FIG. 8.

vi) Broadening of MHC Coverage by Using α-Syn Derived Peptide Immunogen Constructs with Different Promiscuous T Helper Epitopes.

When designing a pharmaceutical composition to treat patients of diverse genetic background, it is important to allow the design to cover maximal population with diverse genetic background. It was therefore explored for synergistic immunogenicity effect of α-Syn derived peptide immunogen constructs for such a combination. Since promiscuous T helper epitopes derived from MVF or HBsAg represent amongst the most potent ones to provide such immunogenicity enhancement, combination of peptide constructs containing the a helper T epitope was therefore designed for such exploration. A mixture of two peptide immunogen constructs with the same B epitopes was found to elicit a respectable immune response when compared to that elicited by the respective individual peptide construct.

### Example 6

### Focused Antibody Response Elicited by α-Syn Peptide Immunogen Constructs to the Targeted B Cell Epitope Only

It is well known that all carrier proteins (e.g. Keyhole Limpet Hemocyanin (KLH) or other carrier proteins such as Diphtheria toxoid (DT) and Tetanus Toxoid (TT) proteins) used to potentiate an immune response directed against the targeted B cell epitope peptide by chemical conjugation of such B cell epitope peptide to the respective carrier protein will elicit more than 90% of the antibodies directed against the potentiating carrier protein and less than 10% of the antibodies directed again the targeted B cell epitope in immunized hosts. It is therefore of interest to assess the specificity of the α-Syn peptide immunogen constructs of the present invention. A series of eight α-Syn peptide immunogen constructs (SEQ ID NOs: 107 to 114) with B cell epitopes of varying lengths that are linked through a spacer sequence to the heterologous T cell epitope UBITh1 (SEQ ID NO: 83) were prepared for immunogenicity assessment. The UBITh1 (T helper peptide used for B epitope immunopotentiation) was coated to the plates and the guinea pig immune sera were employed to test for cross reactivities with the UBITh1 peptide used for immunopotentiation. In contrast to the high immunogenicity of these constructs towards the corresponding targeted B epitopes as illustrated by the high titers of antibodies generated towards the B epitope(s) as shown in Tables 6 and 7, most, if not all, of the immune sera were found non-reactive to the UBITh1 peptide as shown in Table 8.

In summary, simple immunogen design incorporating target B cell epitope linked to carefully selected T helper epitope allows the generation of a focused and clean immune response targeted only to the α-Syn B cell epitope. For pharmaceutical composition design, the more specific the immune response it generates, the higher safety profile it provides for the composition. The α-Syn peptide immunogen constructs of this instant invention is thus highly specific yet highly potent against its target.

### Example 7

### Epitope Mapping for Fine Specificity Analysis by Immune Sera (9 WPI) Against Various Alpha-Synuclein Peptide Immunogen Constructs

In a fine epitope mapping study (Table 9) to determine the antibody binding site(s) to specific residues within the α-Syn C-terminal region, 52 overlapping 10-mer (SEQ ID Nos: 18 to 69) were synthesized, which cover α-Syn amino acid sequence of (K80-A140). Two longer peptides of (97-135, SEQ ID No: 10) and (111-132, SEQ ID No: 17) were employed as positive control. These 10-mer peptides and two longer peptides were individually coated onto 96-well microtiter plate wells as solid-phase immunoabsorbents. The pooled guinea pig antisera were added with 1:100 dilution in specimen diluent buffer to the plate wells coated with 10-mer peptide at 2.0 μg/mL and then incubated for one hour at 37° C. After washing the plate wells with wash buffer, the horseradish peroxidase-conjugated Protein A/G is added and incubated for 30 min. After washing with PBS again, the substrate is added to the wells for measurement of absorbance at 450 nm by ELISA plate reader, which the samples were analyzed in duplicate. The binding of antisera with the corresponding long α-Syn peptide of the B epitope immunogen construct represents the maximal binding.

As shown in Table 9, the pooled 9 wpi guinea pig immune sera obtained respectively from six α-Syn peptide immunogen constructs [(K97-D135, SEQ ID No: 110), (G111-D135, SEQ ID No: 108), (G111-G132, SEQ ID No: 113), (E126-D135, SEQ ID No: 112), (G101-A140, SEQ ID No: 104) and (E126-A140, SEQ ID No: 99)] were selected for fine epitope mapping. These six B epitope fragments of varying lengths fully cover 97-140 sequence of α-Synuclein C-terminal region. ELISA results showed that all six immune sera reacted strongly with the representative α-Syn long peptide (97-135, SEQ ID No: 10). For the 10-mer fine epitope mapping study, the results revealed an immunogenic epitope covering around the region from AA114 to 125 (peptides 114-123, 115-124, 116-125 of SEQ ID Nos: 52, 53 and 54) and a highly immunogenic region at the C-terminal end represented by the peptide 131-140 (SEQ ID NO: 69). Interestingly, most of the immune sera derived from the C-terminal α-Syn peptide immunogen constructs elicited antibodies that recognize, not linear, but conformational epitopes except for one which is located at the α-Syn C-terminus with the sequence of EGYQDYEPEA (SEQ ID NO: 69) and responsible for the cross-reactivity with β-Syn protein.

This epitope mapping finding was less expected but correlated well with the finding that these antibodies derived from the α-Syn peptide immunogen constructs as represented by α-Syn 111-132 (SEQ ID NO: 113) and α-Syn 126-135 (SEQ ID NO: 112) from the C-terminal random coil region of α-Syn that are linked to an heterologous Th epitope structure leading to a conformational structure resembled by a denatured β-sheet of α-Syn, and non-crossreactive with the native α-helix of α-Syn.

### Example 8

### Antibodies Elicited by α-Syn Peptide Immunogen Constructs and Formulations Thereof: Anti-Aggregation and Dis-Aggregation Effects on Recombinant Alpha Synuclein Protein

We evaluated the effects of α-Syn peptide immunogen constructs in in vitro anti-aggregation assays and disaggregation assays by using anti-α-Syn antibodies purified from guinea pig antisera on recombinant α-Syn.

a. Inhibition of α-Syn Aggregation

An initial screening assay of different anti-α-Syn antibodies purified from guinea pigs immunized with different α-Syn peptide immunogen constructs for potential anti-aggregation ability was conducted by quantifying the level of changes of α-Syn aggregations by thioflavin T measurement as described in Example 3. Recombinant α-Syn prepared in PBS at 100 μM were further incubated in 40 μL PBS/KCl buffer (2.5 mM MgCl2, 50 mM HEPES and 150 mM KCl in 1×PBS, pH 7.4) at concentration of 5 μM in a 384-well plate for 6 days to trigger the aggregation. Different concentrations (0.05, 0.5, or 5 μg/mL) of anti-α-Syn antibodies purified from guinea pigs antisera immunized with different α-Syn peptide immunogen constructs, collected at different time points were added in the incubation mixture to evaluate the respectively effects on inhibiting the aggregation of α-Syn. By the end of incubation, the aggregation level was determined using the ThT assays. The readings obtained from each test run were normalized by taking the aggregation level in the Vehicle Control as 100% and taking the readings obtained in the absence of α-Syn as 0%.

As summarized in Table 10, three anti-α-Syn antibodies elicited by α-Syn₁₁₁₋₁₃₂, α-Syn₁₂₁₋₁₃₅, or α-Syn₁₂₆₋₁₃₅ collected at 9 WPI and beyond revealed more potent and concentration-dependent inhibitions on α-Syn aggregation. Of all the anti-α-Syn antibodies assayed, four selected antibodies which were elicited by α-Syn₁₁₁₋₁₃₂ (SEQ ID NO:113), α-Syn₁₂₁₋₁₃₅ (SEQ ID NO:107), α-Syn₁₂₃₋₁₃₅ (SEQ ID NO:111), or α-Syn₁₂₆₋₁₃₅ (SEQ ID NO:112) (collected at 9 WPI) demonstrated an inhibitory effect on α-Syn aggregation by nearly 40% (FIG. 1) compared to aggregation level of the Vehicle Control as 100%.

b. Disassociation of Pre-Formed α-Syn Aggregates

From the above studies it was noted that anti-α-Syn antibodies purified from guinea pigs antisera immunized with certain α-Syn peptide immunogen constructs possessed the effects on inhibition of α-Syn aggregation. To further evaluate if the antibodies elicited by the α-Syn peptide immunogen constructs remained effective in disassociating pre-formed α-Syn aggregates, in vitro disaggregation assays by using anti-α-Syn antibodies purified from guinea pigs antisera were conducted.

The α-Syn was aggregated in 200 μL PBS/KCl buffer at concentration of 5 μM for 3 days. After centrifugation (13,000×g, 4° C., 30 mins), the α-Syn aggregates were harvested and confirmed with the ThT assays. The pre-formed α-Syn aggregates were then incubated in 100 μL PBS/KCl buffer with or without the anti-α-Syn antibodies purified from guinea pigs antisera (5 μg/mL) for 3 days. After incubation, the aggregates were collected after centrifugation of 13,000×g at 4° C. for 30 mins and then quantified with the ThT assay as described in Example 3. The residual α-Syn aggregates after spontaneous disassociations in the Vehicle Control was normalized to 100%.

Two selected anti-α-Syn antibodies which were elicited by α-Syn₁₁₁₋₁₃₂ (SEQ ID NO:113) or α-Syn₁₂₆₋₁₃₅ (SEQ ID NO:112), and the combination of α-Syn₁₁₁₋₁₃₂ (SEQ ID NO:113) elicited and α-Syn₁₂₆₋₁₃₅ (SEQ ID NO:112) elicited anti-α-Syn antibodies were tested in this in vitro disaggregation assay. As a result, anti-α-Syn antibodies elicited by α-Syn₁₂₆₋₁₃₅ (SEQ ID NO:112) and α-Syn₁₁₁₋₁₃₂ (SEQ ID NO:113) demonstrated a disassociating effect on pre-formed α-Syn aggregates by nearly 50% when compared to the Vehicle Control as 100%, while the other anti-α-Syn antibodies and the antibodies purified from pre-immunized animals failed to show the comparable effects (FIG. 2).

### Example 9

### Antibodies Elicited by α-Syn Peptide Immunogen Constructs and Formulations Thereof: Anti-Aggregation and Dis-Aggregation Effects on α-Syn Aggregation Kinetics in α-Syn-Overexpressing Cells

It is known that α-Syn aggregation accelerates during neuronal differentiation. In order to assess the effects of the α-Syn peptide immunogen constructs on either inhibiting α-Syn aggregation or disassociating pre-formed α-Syn aggregates in a cell-based condition, anti-α-Syn antibodies purified from guinea pigs antisera immunized with different α-Syn peptide immunogen constructs were evaluated with the NGF-treated, neuronal-differentiating α-Syn-overexpressing PC12 cells-based anti-aggregation assays and disaggregation assays.

a. Inhibition of α-Syn Aggregation

α-Syn-overexpressing PC12 cells were seeded onto poly-D-lysine pre-coated 96-well plates and then treated with nerve growth factor (NGF) (100 ng/mL) along with anti-α-Syn antibodies purified from guinea pigs immunized with different α-Syn peptide immunogen constructs (0 or 0.5 μg/mL) for 4 days in order to validate the anti-aggregation activities.

The treated cells were lysed and 20 μg of cell lysates were separated by SDS-PAGE and then detected with α-Syn antibody (BD). The amount of detected α-Syn signals in higher molecular weight region was quantified and then normalized to Vehicle Control group as 100%. As shown in FIG. 3, inhibitory effects on the amount of aggregated α-Syn up to 80 to 90% were observed among all four selected anti-α-Syn antibodies elicited by α-Syn₁₁₁₋₁₃₂ (SEQ ID NO:113), α-Syn₁₂₁₋₁₃₅ (SEQ ID NO:107), α-Syn₁₂₃₋₁₃₅ (SEQ ID NO:111), or α-Syn₁₂₆₋₁₃₅ (SEQ ID NO:112) α-Syn peptide immunogen constructs, compared to the amount of aggregated α-Syn in Vehicle Control.

b. Disassociation of Pre-Formed α-Syn Aggregates

In order to validate the disaggregation activities on pre-formed α-Syn aggregates, α-Syn-overexpressing PC12 cells were treated with NGF (100 ng/mL) for 3 days for neuronal differentiation to initiate the aggregation of α-Syn, before further treated with anti-α-Syn antibodies purified from guinea pigs immunized with different α-Syn peptide immunogen constructs (0 or 0.5 μg/mL) for another 4 days.

The treated cells were lysed and 20 μg of cell lysates were separated by SDS-PAGE and then detected with α-Syn antibody (BD). The amount of detected α-Syn signals in higher molecular weight region was quantified and then normalized to Vehicle Control group as 100%. As also shown in FIG. 3, 50 to 60% decrease in the amount of aggregated α-Syn was observed in anti-α-Syn antibodies elicited by α-Syn₁₂₁₋₁₃₅ (SEQ ID NO:107), α-Syn₁₂₃₋₁₃₅ (SEQ ID O:111), or α-Syn₁₂₆₋₁₃₅ (SEQ ID NO:112) peptide immunogen constructs, while anti-α-Syn antibodies elicited by α-Syn₁₁₁₋₁₃₂ (SEQ ID NO:113) demonstrated a higher than 90% decrease in the amount of aggregated α-Syn.

### Example 10

### Antibodies Elicited by α-Syn Peptide Immunogen Constructs and Formulations Thereof: Effect on Reduction of Microglial TNF-α and IL-6 Secretion

It is believed that nigral neuronal damage releases aggregated α-Syn into substantia nigra, which activates microglia with production of proinflammatory mediators, thereby leading to persistent and progressive nigral neurodegeneration in PD. To assess the effects in reducing microglia activation by anti-α-Syn antibodies purified from guinea pigs immunized with different α-Syn peptide immunogen constructs, the amount of proinflammatory mediators, TNF-α (tumor necrosis factor alpha) and IL-6 (interleukin-6), released by microglias upon treatment with α-Syn aggregates in the presence or absence of different anti-α-Syn antibodies were measured.

Murine BV2 cells or human SVG p12 cells were seeded at 5,000 cells/well in RPMI 1640 medium supplemented with 1% FBS. The cells were treated with 1 μM α-Syn and incubated at 37° C., 5% CO₂ in a humidified atmosphere for 24 hrs. After which, the culture medium was collected, centrifuged, and the supernatants were isolated. The concentrations of IL-6 secreted by BV2 cells and TNF-α secreted by SVG p12 cells in the supernatants were analyzed in triplicate by using mouse IL-6 or human TNF-α mouse ELISA kits (Thermofisher), respectively. The signal was normalized to Vehicle Control as 100%.

The data showed that the anti-α-Syn antibodies elicited by α-Syn₁₁₁₋₁₃₂ (SEQ ID NO:113) and α-Syn₁₂₃₋₁₃₅ (SEQ ID NO:111) reduced the α-Syn aggregates-mediated TNF-α release by SVG p12 cells by 30 to 50%, while the anti-α-Syn antibodies elicited by α-Syn₁₂₃₋₁₃₅ (SEQ ID NO:111) reduced the IL-6 release by SVGP12 cells by around 30% (FIG. 4). The results indicated that the anti-α-Syn antibodies elicited by α-Syn₁₂₃₋₁₃₅ (SEQ ID NO:111) were more potent than the other anti-α-Syn antibodies tested in mitigating α-Syn aggregates-mediated microglial activation.

### Example 11

### Antibodies Elicited by α-Syn Peptide Immunogen Constructs and Formulations Thereof: Effect on Reduction of Neurodegeneration Triggered by Exogenous Alpha Synuclein

In order to assess the neuroprotective effects of anti-α-Syn antibodies purified from guinea pig antisera immunized with different α-Syn peptide immunogen constructs, an in vitro neurodegeneration model with exogenous, pre-formed α-Syn aggregates on NGF-treated, neuronal-differentiated PC12 cells was adopted.

PC12 cells were treated with NGF (100 ng/mL) for 6 days to induce neuronal differentiation. The morphology of the neuronal-differentiated cells were confirmed and analyzed by InCell high-content Image analysis system (GE Healthcare). The neurotrophic effects of NGF reflected on neurite outgrowth and the number of neuronal-differentiated cells were quantified. The levels of neurite outgrowth and the number of neuronal-differentiated cells were shown in percentages (mean±SEM) after normalization. The neurite length of PC12 cells with and without NGF treatment were taken as 100% and 0%, respectively. The number of neuronal-differentiated PC12 cells upon 6 days of NGF treatment was normalized to 100%.

Neurodegeneration was observed by adding exogenous, pre-formed α-Syn aggregates onto the neuronal-differentiated PC12 cells. In the presence of pre-formed α-Syn aggregates, the neurite length was shortened and the number of cells was decreased in the neuronal-differentiated PC12 cells. This α-Syn aggregates-driven neurodegeneration was proportional to the amount of exogenous α-Syn aggregates added, and could be blocked by curcumin, widely known for its neuroprotective effects against neurotoxicity of α-Syn aggregates, in a concentration dependent manner. The commercially available anti-α-Syn antibodies (BD bioscience), but not the antibodies purified from naïve guinea pigs, attenuated the α-Syn aggregates-driven neurodegeneration. This model was adopted as a screening platform to identify which anti-α-Syn antibodies purified from guinea pig antisera immunized with different α-Syn peptide immunogen constructs possessed the neuroprotective effects in restoring the neurite growth as well as neuronal survival in a concentration-dependent manner (Tables 11 and 12).

As a result, the anti-α-Syn antibodies purified from guinea pig antisera immunized with more than half of the different α-Syn peptide immunogen constructs restored the neurite growth concentration-dependently (Table 11), and the anti-α-Syn antibodies purified from guinea pig antisera immunized with almost all of the different α-Syn peptide immunogen constructs protected neuronal-differentiated PC12 cells from α-Syn aggregates-triggered neuronal death (Table 12). Taken the two different parameters together, it was found that nearly one third of the anti-α-Syn antibodies assayed possessed the effects on both the neurite length and the survival of cells against the neurotoxicity of α-Syn aggregates. The anti-neurodegenerative effects of the anti-α-Syn antibodies elicited by α-Syn₁₁₁₋₁₃₂ (SEQ ID NO:113), α-Syn₁₂₆₋₁₃₅ (SEQ ID NO:112), and the preimmune antibodies from naïve guinea pigs were observed and quantified for the length of neurites and the number of cells with calcein AM (Life Technologies), a fluorescent live-cell labeling dye. It was shown that in the neurite-rich neuronal-differentiated PC12 cells, anti-α-Syn antibodies elicited by α-Syn₁₁₁₋₁₃₂ (SEQ ID NO:113) (FIG. 5B) and α-Syn₁₂₆₋₁₃₅ (SEQ ID NO:112) (FIG. 5C), but not preimmune antibodies purified from naïve guinea pigs (FIG. 5A), exhibited protective effects on α-Syn aggregates-mediated shortening of neurite length.

### Example 12

### Antibodies Elicited by α-Syn Peptide Immunogen Constructs and Formulations Thereof: Effect on Reduction of Neurodegeneration in α-Syn Overexpressing Cells

In order to assess the neuroprotective effects of anti-α-Syn antibodies purified from guinea pig antisera immunized with different α-Syn peptide immunogen constructs, in vitro neurodegeneration models with wild-type α-Syn-overexpressing PC12 cells and A53T mutated α-Syn-overexpressing PC12 cells were adopted.

After incubation with NGF, the mock-controlled cells (transfected with plasmid vector) developed long neurite extension and increased in cell numbers similarly to the parental wild-type PC12 cells, while the wild-type α-Syn-overexpressing PC12 cells and the A53T mutated α-Syn-overexpressing PC12 cells failed to develop comparable neurite extension or increase in cell numbers, confirming the neurodegenerative effects accompanied with aggregated α-Syn upon NGF treatment. In characterization of overexpressed α-Syn in the wild-type α-Syn-overexpressing PC12 cells upon NGF treatment, western blotting and ThT assays was carried out with the cell lysates of the wild-type α-Syn-overexpressing PC12 cells after NGF treatment. The western blotting result demonstrated that overexpressed α-Syn in the cell lysates of the wild-type α-Syn-overexpressing PC12 cells upon NGF treatment was oligomeric, and the ThT assay results indicated that α-Syn in the cell lysate of the wild-type α-Syn-overexpressing PC12 cells upon NGF treatment were of β-sheet structure (i.e., elevated ThT fluorescence signals). Compared to the western blotting and ThT assay results of the wild-type α-Syn-overexpressing PC12 cells without NGF treatment, it was suggested that an α-helix-to-β-sheet structural transition of overexpressed α-Syn occurred upon NGF-induced neuronal differentiation, which might subsequently bring forth the neurodegenerative effects of the β-sheet oligomeric α-Syn. In addition, compared to the wild-type α-Syn-overexpressing PC12 cells, overexpressed A53T mutated α-Syn resulted in stronger neurodegenerative effects reflected in both shortened neurite length and decreased number of cells upon NGF treatment, indicating that A53T mutated α-Syn triggered stronger neurodegenerative effects than wild-type α-Syn in the α-Syn-overexpressing PC12 cells.

Anti-α-Syn antibodies elicited by α-Syn₁₀₁₋₁₃₂ (SEQ ID NO:114), α-Syn₁₁₁₋₁₃₂ (SEQ ID NO:113), α-Syn₁₂₁₋₁₃₅ (SEQ ID NO:107), α-Syn₁₂₃₋₁₃₅ (SEQ ID NO:111), or α-Syn₁₂₆₋₁₃₅ (SEQ ID NO:112), and the combination of anti-α-Syn antibodies elicited by α-Syn₁₁₁₋₁₃₂ (SEQ ID NO:113) and α-Syn₁₂₆₋₁₃₅ (SEQ ID NO:112) were assayed by the in vitro neurodegeneration model with wild-type α-Syn-overexpressing PC12 cells to evaluate their individual protective effects against neurodegeneration. The wild-type α-Syn-overexpressing PC12 cells were treated with NGF for 3 days to initiate the neuronal differentiation, before being incubated with both the anti-α-Syn antibodies (of a final concentration of 5 μg/mL) and NGF for additional 3 days. The microscopical observation of the cells by the end of the incubation period revealed restored neurite length and increased number of cells upon co-incubation with the selected anti-α-Syn antibodies, compared to the Vehicle Control. Quantification of the neurite length and the number of cells was made with the readings of the parental PC12 cells treated with NGF for 6 days normalized to 100%. As a result, anti-α-Syn antibodies elicited by α-Syn₁₀₁₋₁₃₂ (SEQ ID NO:114), α-Syn₁₁₁₋₁₃₂ (SEQ ID NO:113), or α-Syn₁₂₃₋₁₃₅ (SEQ ID NO:111), and the combination of anti-α-Syn antibodies elicited by α-Syn₁₁₁₋₁₃₂ (SEQ ID NO:113) and α-Syn₁₂₆₋₁₃₅ (SEQ ID NO:112) showed significantly larger number of cells, while anti-α-Syn antibodies elicited by α-Syn₁₀₁₋₁₃₂ (SEQ ID NO:114), α-Syn₁₁₁₋₁₃₂ (SEQ ID NO:113), α-Syn₁₂₃₋₁₃₅ (SEQ ID NO:111), or α-Syn₁₂₆₋₁₃₅ (SEQ ID NO:112), and the combination of anti-α-Syn antibodies elicited by α-Syn₁₁₁₋₁₃₂ (SEQ ID NO:115) and α-Syn₁₂₆₋₁₃₅ (SEQ ID NO:114) showed significant longer neurite length, when compared to the Vehicle Control (FIGS. 6A and 6B).

### Example 13

### Antibodies Elicited by α-Syn Peptide Immunogen Constructs and Formulations Thereof: Specificity to Beta-Sheet Oligomeric and Fibrillar Alpha Synuclein Protein

In order to better characterize the specificity of anti-α-Syn antibodies purified from guinea pig antisera immunized with different α-Syn peptide immunogen constructs, a series of in vitro assays were conducted on different sizes of the α-Syn molecular complex, different amyloidogenic proteins including α-Syn, Aβ, and tau protein, and aggregated α-Syn in α-Syn-overexpressing PC12 cells upon NGF treatment.

a. Specificity to Larger α-Syn Molecular Complexes

Western blotting of α-Syn molecular complexes of different sizes was carried out using anti-α-Syn antibodies purified from guinea pig antisera immunized with different α-Syn peptide immunogen constructs as primary antibodies. The results showed that all anti-α-Syn antibodies reacted strongly with α-Syn molecular complexes of larger sizes, including dimers, trimers, tetramers, and oligomers, in addition to the smaller-sized monomeric α-Syn. When compared to the commercially available anti-α-Syn antibody, Syn211 (Abcam), anti-α-Syn antibodies elicited by α-Syn₁₁₁₋₁₃₂ (SEQ ID NO:113), α-Syn₁₂₁₋₁₃₅ (SEQ ID NO:107), α-Syn₁₂₃₋₁₃₅ (SEQ ID NO:111) and α-Syn₁₂₆₋₁₃₅ (SEQ ID NO:112) demonstrated a higher ratio of the signal of α-Syn molecular complexes of larger sizes (including dimers, trimers, tetramers, and oligomers) to the signal of the smaller-sized monomeric α-Syn (FIGS. 7A and 7B), suggesting the anti-α-Syn antibodies possessed specificity to larger α-Syn molecular complexes.

b. Specificity to α-Syn Among Different Amyloidogenic Proteins

Dot blot assays with different species (i.e., the α-helix monomers, β-sheet monomers, β-sheet oligomers and β-sheet fibrils) of different amyloidogenic proteins (i.e., α-Syn, Aβ₁₋₄₂ and Tau441) prepared as described in Example 3 were carried out using anti-α-Syn antibodies purified from guinea pig antisera immunized with different α-Syn peptide immunogen constructs as primary antibodies. The resulted showed that the anti-α-Syn antibodies elicited by α-Syn₁₂₆₋₁₃₅ (SEQ ID NO:112) and α-Syn₁₁₁₋₁₃₂ (SEQ ID NO:113) reacted specifically to all the β-sheet forms (monomeric, oligomeric and fibrillar species) of α-Syn, but not to the α-helix monomers (FIGS. 8A, 8B, and 8C). Moreover, the anti-α-Syn antibodies elicited by α-Syn₁₂₆₋₁₃₅ (SEQ ID NO:112) and α-Syn₁₁₁₋₁₃₂ (SEQ ID NO:113) reacted more strongly to the β-sheet fibrils and the β-sheet oligomers of α-Syn than to the β-sheet monomers of α-Syn. In contrast, the anti-α-Syn antibodies elicited by α-Syn₁₂₆₋₁₃₅ (SEQ ID NO:112) and α-Syn₁₁₁₋₁₃₂ (SEQ ID NO:113) showed no detected reactivity to β-Syn or the different species (i.e., the α-helix monomers, β-sheet monomers, β-sheet oligomers and β-sheet fibrils) of amyloidogenic proteins Aβ₁₋₄₂ and Tau441 (FIGS. 8A, 8B, and 8C). The findings suggested that the anti-α-Syn antibodies elicited by α-Syn₁₂₆₋₁₃₅ (SEQ ID NO:112) and α-Syn₁₁₁₋₁₃₂ (SEQ ID NO:113) possessed the specificity to α-Syn of β-sheet monomeric, β-sheet oligomeric and β-sheet fibrillar forms.

c. Binding Specificity to Aggregated α-Syn in α-Syn-Overexpressing PC12 Cells Upon NGF Treatment

Immunocytochemistry (ICC) with anti-α-Syn antibodies purified from guinea pigs antisera immunized with different α-Syn peptide immunogen constructs was carried out on parental PC12 cells, mock-controlled PC12 cells, wild-type α-Syn-overexpressing PC12 cells, and A53T mutated α-Syn-overexpressing PC12 cells to evaluate the binding affinity of the antibodies to aggregated α-Syn upon NGF treatment, as described in Example 3. As the quantification result showed in FIG. 9, anti-α-Syn antibodies elicited by α-Syn₁₁₁₋₁₃₂ (SEQ ID NO:113), α-Syn₁₂₁₋₁₃₅ (SEQ ID NO:107), or α-Syn₁₂₆₋₁₃₅ (SEQ ID NO:112) demonstrated a stronger reactivity in the wild-type α-Syn-overexpressing PC12 cells and the A53T mutated α-Syn-overexpressing PC12 cells than in the parental PC12 cells or the mock-controlled PC12 cells upon NGF treatment. As the overexpressed α-Syn aggregation was induced upon NGF treatment, the findings suggest that the anti-α-Syn antibodies elicited by α-Syn₁₁₁₋₁₃₂ (SEQ ID NO:113), α-Syn₁₂₁₋₁₃₅ (SEQ ID NO:107), or α-Syn₁₂₆₋₁₃₅ (SEQ ID NO:112) possessed the specificity to aggregated α-Syn in the wild-type α-Syn-overexpressing PC12 cells and A53T mutated α-Syn-overexpressing PC12 cells upon NGF treatment.

### Example 14

### Immunohistochemical Staining of Human Brain with Parkinson's Disease for Assessment of Tissue Specificity of the α-Syn Peptide Immunogen Constructs and Formulations Thereof

Immunohistopathology study using preimmune, anti-α-Syn antibodies elicited by α-Syn₁₂₆₋₁₃₅ (SEQ ID NO:112) or α-Syn₁₁₁₋₁₃₂ (SEQ ID NO:113), and the 1:1 combination of both anti-α-Syn antibodies was performed on the normal human tissues in order to monitor for specificity and undesirable antibody autoreactivities. The panel of human tissues (Pantomics) was deparaffinized with xylene, rehydrated in ethanol, and then treated with 0.25% trypsin solution with 0.5% CaCl₂ in PBS for 30 min and incubated in 1% hydrogen peroxide in methanol to block endogenous peroxidase activity followed by incubation with 10% Block Ace (Sigma) in PBS, before the anti-α-Syn antibodies from guinea pigs immunized with α-Syn₁₂₆₋₁₃₅ (SEQ ID NO:112) or α-Syn₁₁₁₋₁₃₂ (SEQ ID NO:113) and the 1:1 combination of both antibodies (1:300 dilution) were applied. The sections were developed with 3-3′diaminobenzidine (DAB) and were counter-stained with hematoxylin before being examined microscopically. In contrast to the positive reactivity of the commercial anti-α-Syn antibody (BD, 610708), the anti-α-Syn antibodies purified from guinea pigs immunized with α-Syn₁₂₆₋₁₃₅ (SEQ ID NO:112) or α-Syn₁₁₁₋₁₃₂ (SEQ ID NO:113) and the 1:1 combination of both antibodies showed negative reactivity to normal human tissues, which was compatible to the pattern of the preimmune antibodies from naïve guinea pigs (FIG. 10A). Another immunohistopathology study using preimmune, anti-α-Syn antibodies elicited by α-Syn₁₂₆₋₁₃₅ (SEQ ID NO:112) or α-Syn₁₁₁₋₁₃₂ (SEQ ID NO:113), and the 1:1 combination of both anti-α-Syn antibodies was performed to test their reactivity with human Parkinson's disease brain. Tissue sections of three regions (i.e., cerebellum, corpus callosum and thalamus) (BioChain) were assayed. As a result, anti-α-Syn antibodies elicited by α-Syn₁₂₆₋₁₃₅ (SEQ ID NO:112) or α-Syn₁₁₁₋₁₃₂ (SEQ ID NO:113), and the 1:1 combination of both anti-α-Syn antibodies showed positive reactivity (with pointing arrows) to the PD brain sections in all three regions, in comparison with the negative reactivity in health brain sections (FIGS. 10B and 10C). Quantification of the reactivity to the α-Syn aggregates in the PD brain sections was done by counting the positive stains under microscopical observation. The results showed that anti-α-Syn antibodies elicited by α-Syn₁₂₆₋₁₃₅ (SEQ ID NO:112) or α-Syn₁₁₁₋₁₃₂ (SEQ ID NO:113), and the 1:1 combination of both anti-α-Syn antibodies had strong positivity in the PD brain sections, compared to the healthy human brain sections. And of the three different anti-α-Syn antibodies assayed, the antibodies elicited by α-Syn₁₁₁₋₁₃₂ (SEQ ID NO:113) had the strongest immunoreactivity to the α-Syn aggregates in the PD brain sections.

### Example 15

### Proof of Efficacy for the α-Syn Peptide Immunogen Constructs and Formulations Thereof in Animal Models

a. Immunization and Blood/Brain Tissue Collection

Parkinson' Disease (PD) mouse models were established as described in Example 4. Two weeks after MPP⁺ injection, or 7 weeks after fibrillar α-Syn-inoculation, mice were randomly divided into to three groups including UBITh1-linked α-Syn₁₁₁₋₁₃₂ (SEQ ID NO:113) peptide, UBITh1-linked α-Syn₁₂₆₋₁₃₅ (SEQ ID NO: 112) peptide, and the combination of both peptides, in addition to the Adjuvant Group (immunized with the adjuvants and solvent used in the preparation of the compositions (ISA 51 VG, CpG3, 0.2% TWEEN®-80)). Intramuscular (IM) immunization were administrated for three times with an interval of 3 weeks, at the dose of 40 μg. The administration and blood collection schedules were carried out according to the Table 13.

At each time point, 200 μL of blood was drawn via facial vein blood sampling. Blood dripping from the punctured submandibular vein was collected into a microtube and the serum was prepared by centrifugation at 300 rpm for 10 minutes. After animal sacrifice, brain tissue samples were collected for western blotting.

b. Immune Response in PD Model Mice Receiving Compositions Containing of α-Syn₁₁₁₋₁₃₂ (SEQ ID NO: 113) or/and α-Syn₁₂₆₋₁₃₅ (SEQ ID NO: 112) Peptide Immunogen Constructs

Pooled serum samples of each treatment group were diluted in 1% BSA (in PBST) and then applied to the ELISA plate coated with 200 μL of α-Syn full length peptide (Cloud-clone) in 0.1 M sodium bicarbonate (α-Syn concentration 4.4 μg/μl, pH 9.6). After 2 hours of incubation at room temperature and three washes with PBST, 100 μl of HRP-conjugated anti-mouse IgG antibody diluted in 1:3000 with 1% BSA were added to react for 2 hours at room temperature. After which, the plates were washed three times with PBST and incubated with 100 μl of 3,3,5,5-tetramethylbenzidine (TMB) for 10 minutes in dark. 100 μL of 2M H₂SO₄ was then applied and incubated for 15 to 30 minutes before the optical density (OD) value at 450 nm was measured with SpectraMax i3x Multi-Mode Detection (Molecular Devices).

The two PD murine models immunized with the α-Syn₁₁₁₋₁₃₂ (SEQ ID NO:113) formulated, the α-Syn₁₂₆₋₁₃₅ (SEQ ID NO: 112) formulated, or the combination of both peptide immunogen constructs had anti-α-Syn antibody optical density (OD) value greater than 3.0 after the second immunization, which remained elevated by the time of study termination at 15 or 19 weeks post-initial immunization, in the MPP⁺ induced model (FIG. 11A) or fibrillar α-Syn-inoculated model (FIG. 11B), respectively, while adjuvant-administered animals did not elicit measurable anti-α-Syn immune response.

It is noted that in the fibrillar α-Syn-inoculated model, the α-Syn₁₁₁₋₁₃₂ construct elicited stronger immune response than the α-Syn₁₂₆₋₁₃₅ construct (FIG. 11B), while the difference in immunogenicity wasn't observed in the MPP⁺ induced model (FIG. 11A).

c. Reduction in Serum α-Syn Level

The α-Syn levels of the pooled serum from animals of each group were assayed using ELISA kit (SEB222Mu, USCN) which could detect both alpha helix and β-sheet α-Syn described in Example 3.

The α-Syn quantitative ELISA was to test whether the anti-α-Syn antibody response of the immunized groups was associated with a reduced amount of peripheral α-Syn when compared to the untreated animals. It was shown that immunization with the α-Syn₁₂₆₋₁₃₅ (SEQ ID NO:112), α-Syn₁₁₁₋₁₃₂ (SEQ ID NO:113), or the combination of these constructs had decreased optical density (OD) values of α-Syn levels when compared to the adjuvant-administered animals, in both MPP⁺ induced model (FIG. 12A) and fibrillar α-Syn-inoculated model (FIG. 12B). The results suggested that with the generation of anti-α-Syn antibody response upon immunization with α-Syn peptide immunogen constructs, the amount of α-Syn in the peripheral circulation was decreased accordingly.

d. Reduction in Oligomeric α-Syn Level in Brain

After animal sacrifice, brain tissue samples were collected for western blotting. For MPP+ induced mice, the brain was removed and homogenized, while for the fibrillar α-Syn-inoculated mice, the striatum and substantia nigra regions were isolated first and then homogenized. The brain tissue lysate was prepared by adding lysis buffer (Amresco) and 1× proteinase inhibitor (Roche) into the homogenate. The lysate was then separated by 10% SDS-PAGE (sodium dodecyl sulphate-polyacrylamide gel electrophoresis), transferred onto polyvinylidene difluoride (PVDF) membrane, and incubated overnight with 5% milk in PBS. To detect the abundance of dopaminergic neurons, the membranes were incubated with anti-tyrosine hydroxylase antibody (dilution 1:1000, Abcam), followed by hybridized with the goat anti-rabbit IgG (H+L) HRP-conjugated secondary antibody (1:5000 dilution, Jackson Immunoresearch). For visualization, Luminata Western HRP Substrates was used and the resulted signal was captured with ChemiDoc-It 810 digital image system. Quantification of oligomeric α-Syn level was done by normalized with the GAPDH level, and the ratio of non-lesioned lysate was further standardized to 100% for comparison.

In the MPP⁺ induced model, the reduction in the oligomeric α-Syn fraction was shown in the animals immunized with α-Syn₁₁₁₋₁₃₂ peptide immunogen construct (FIG. 13A). Similarly, in the fibrillar α-Syn-inoculated mice, western blotting with lysates of the substantia nigra and also striatum of the ipsilateral side as the fibrillar α-Syn-inoculation (FIGS. 14A and 14D) and with the lysates of the striatum of the contralateral side of fibrillar α-Syn-inoculation (FIG. 14F) showed that the up to 2- to 3-fold increased oligomeric α-Syn level seen in the adjuvant control mice was mitigated after treatment with α-Syn₁₁₁₋₁₃₂ (SEQ ID NO:113)-formulated and with the α-Syn₁₂₆₋₁₃₅ (SEQ ID NO:112) construct. Quantification of the western blotting results was presented in FIGS. 13B, 14B, 14C, 14D and 14G.

e. Reduction in Neuropathology

For the fibrillar α-Syn-inoculated mice, the substantia nigra regions were isolated first and then homogenized. The tissue lysate was prepared by adding lysis buffer (Amresco) and 1× proteinase inhibitor (Roche) into the homogenate. The lysate was then separated by 10% SDS-PAGE (sodium dodecyl sulphate-polyacrylamide gel electrophoresis), transferred onto polyvinylidene difluoride (PVDF) membrane, and incubated overnight with 5% milk in PBS. To detect the abundance of dopaminergic neurons, the membranes were incubated with anti-tyrosine hydroxylase antibody (dilution 1:1000, Abcam), followed by hybridized with the goat anti-rabbit IgG (H+L) HRP-conjugated secondary antibody (1:5000 dilution, Jackson Immunoresearch). For visualization, Luminata Western HRP Substrates was used and the resulted signal was captured with ChemiDoc-It 810 digital image system. The expression level of α-Syn was standardized to GAPDH (glyceraldehyde 3-phosphate dehydrogenase) used as the protein loading control.

The results demonstrated that immunization with the α-Syn₁₁₁₋₁₃₂ construct restored the amount of tyrosine hydroxylase to a level equivalent to that of non-lesioned normal animals (FIGS. 14C-14D), suggesting the neuroprotective effect of the α-Syn peptide immunogen constructs against the neurotoxicity associated with aggregated α-Syn inoculated to the mice.

f. Recovery of Motor Activities

The CatWalk™ XT (Noldus information Technology, Wageningen, Netherlands) is a video-based analysis system used to objectively measure various aspects of footfalls in a dynamic manner, based on the position, pressure, and surface area of each footfall. All mice were trained to cross the runway in a consistent manner at least three times a day before experimentation. A successful run was defined as an animal ran through the runway without interruption or hesitation, and mice that failed the training were excluded from the study.

An average of 5 crossings of each mouse was analyzed. Since the fibrillar α-Syn inoculation was performed on the right brain, left hind feet stand time was considered a reference parameter, alone with the run duration.

In the fibrillar α-Syn-inoculated model, significant difference in the measurement of Left Hindlimb Stand time was seen after treatment with compositions containing α-Syn₁₂₆₋₁₃₅ (SEQ ID NO:112) or α-Syn₁₁₁₋₁₃₂ (SEQ ID NO:113) (FIG. 15A). Meanwhile, in both fibrillar α-Syn-inoculated model and MPP+ induced model, significant difference in the measurement of Run Duration was seen after treatment with compositions containing α-Syn₁₁₁₋₁₃₂ (SEQ ID NO:113) (FIGS. 15B and 15C). The results suggested an association of treatment with α-Syn₁₂₆₋₁₃₅ (SEQ ID NO:112)-formulated or α-Syn₁₁₁₋₁₃₂ (SEQ ID NO:113)-formulated α-Syn peptide immunogen constructs and the improvement in the motor functions of the two PD mouse models.

### Example 16

### Reactivities of Antibodies Generated by the α-Syn Peptide Immunogen Constructs with Different α-Syn Strains Found in Neurodegenerative Disease

α-Syn drives Parkinson's and other synucleinopathies. The α-Syn protein is able to form distinct types of aggregates that have different sizes and structures, and different effects on cells, so that each of these diseases is driven by one or more different types of aggregate. Differently shaped α-Syn aggregates can cause different patterns of damage in the brain and can even cause distinct brain diseases. This study was designed to assess how the antibodies generated by the α-Syn peptide immunogen constructs interact with different α-Syn strains found in neurodegenerative diseases.

Dr. Ronald Melki was a collaborator of this study. Serval distinct types of α-Syn aggregates were produced in the lab that include (a) fibril—a long, twisted, zippered-together strand of α-Syn proteins; (b) ribbon—a broader, flatter structure, and (c) α-Syn oligomers (O550), dopamine stabilized (ODA) and glutaraldehyde stabilized (OGA) oligomers.

Antibodies generated in guinea pigs by various α-Syn peptide immunogen constructs of disclosed herein were tested for their relative affinities. Representative samples from PD-021514 (α-Syn₈₅₋₁₄₀, wpi 08), PD-021522 (α-Syn₈₅₋₁₄₀, wpi 13), PD-100806 (α-Syn₁₂₆₋₁₃₅, wpi 09), PRX002, and a commercial monoclonal antibody Syn1 (clone 42) were tested on distinct α-Syn assemblies including: fibrils, ribbons, fibrils 65, fibrils 91, fibrils 110, on fibrillar assembly pathway α-Syn oligomers (O550), dopamine stabilized (ODA) and glutaraldehyde stabilized (OGA) oligomers, along with a control monomer using a filter trap assay.

### Methods and Materials

a. Assembly of α-Syn into Fibrils and Ribbons

For fibril formation, soluble WT α-Syn was incubated in buffer A (50 mM Tris-HCl, pH 7.5, 150 mM KCl) at 37° C. under continuous shaking in an Eppendorf Thermomixer set at 600 r.p.m. Assembly was monitored continuously in a Cary Eclipse spectrofluorimeter (Varian Inc., Palo Alto, Calif., USA) in the presence of Thioflavin T (15 μM) in 1×1 cm cuvettes under agitation (100 r.p.m.) using a magnetic stir bar (6×3 mm) with an excitation wavelength set at 440 nm and emissions wavelengths set at 440 nm and 480 nm, and an averaging time of 1 s. For ribbon formation, WT α-Syn was dialysed 16 h against 1,000 volume of buffer B (5 mM Tris-HCl pH 7.5) at 4° C., then incubated at 37° C. under continuous shaking in an Eppendorf Thermomixer set at 600 r.p.m. Assembly was monitored by the measurement of the scattered light at 440 nm. Alternatively, the amount of protein remaining in the supernatant after sedimentation at 35,000×g was determined by measurement of the absorbance at 280 nm in a Hewlett Packard 8453 diode array spectrophotometer. The nature of the oligomeric species was assessed using a Jeol 1400 (Jeol Ltd.) TEM following adsorption of the samples onto carbon-coated 200-mesh grids and negative staining with 1% uranyl acetate. The images were recorded with a Gatan Orius CCD camera (Gatan). The ability of α-Syn assemblies to bind Congo red was assessed as follows: α-Syn fibrils and ribbons were incubated for 1 h with 100 μM Congo Red (Sigma-Aldrich, St Louis, Mo., USA) in 20 mM Tris buffer (pH 7.5). The polymers were then sedimented at 20° C. in a TL100 Tabletop Beckman ultracentrifuge (Beckman Instruments, Inc., Fullerton, Calif., USA) at 25,000 g for 30 min. The pellets were washed four times using an equal volume of water. Following resuspension of the pellets an aliquot was placed on a glass coverslip and imaged immediately or allowed to dry. Samples were viewed in bright field and cross-polarized light by polarization microscopy using a Leica (MZ12.5) microscope equipped with cross-polarizers (Leica Microsystems, Ltd., Heerbrugg, Switzerland).

b. Determination of α-Syn Fibril and Ribbon Concentrations

The length heterogeneity of α-Syn fibrils and ribbons was reduced by sonication for 20 min on ice in 2-ml Eppendorf tubes in a VialTweeter powered by an ultrasonic processor UIS250v (250 W, 2.4 kHz; Hielscher Ultrasonic, Teltow, Germany) set at 75% amplitude, 0.5 s pulses. The sedimentation velocities of α-Syn fibrils and ribbons were measured. The sedimentation boundaries were analysed with Sedfit software, using the least squares boundary modelling ls-g*(s), which is best suited for heterogeneous mixtures of large particles. This yielded a distribution of particles with sedimentation coefficients ranging from 50 to 150 S for α-Syn ribbons, from 100 to 1,000 S for α-Syn fibrils, centered on species that have sedimentation coefficient of ˜90 S and 375 S for α-Syn ribbons and fibrils, respectively, corresponding to particles that have a molecular weight of ˜11,500 kDa, for example, made of ˜800 α-Syn molecules (12,000 kDa/14.5 kDa) for α-Syn ribbons, ˜102,000 kDa, for example, made of ˜7,000 α-Syn molecules (10,2000, kDa/14.5 kDa) for α-Syn fibrils. Thus, at the working concentration of 20 μM, the overall particle concentrations of α-Syn ribbons and fibrils are 20 μM/˜800=˜0.02 μM, 20 μM/˜7,000=˜0.003 μM for α-Syn ribbons and fibrils, respectively, given that 100% of α-Syn is assembled in ribbons or fibrils at a steady state, as 100% of the protein is found in the pellet fraction upon centrifugation of the samples.

c. Assessment of the Affinity of Endobodies on Different α-Syn Fibrils and Ribbons

The affinity of antibodies generated by α-Syn peptide immunogen constructs disclosed herein were evaluated for distinct α-Syn assemblies using a filter trap assay with antibody as a reference. The α-Syn assemblies (fibrils, ribbons, fibrils 65, fibrils 91, fibrils 110, on fibrillar assembly pathway α-Syn oligomers (0550), dopamine stabilized (ODA) and glutaraldehyde stabilized (OGA) oligomers) are described in Bousset L. et al., 2013 Nat Commun 4:2575; Makky A. et al., 2016 Sci Rep 6:37970; and Pieri L. et al, 2016 Sci. Rep 6:24526. A control monomeric α-Syn was also used.

Increasing amounts of fibrillar, oligomeric or monomeric α-Syn, in the range from 20 pg to 200 ng, were spotted on nitrocellulose filters using a slot blot filtration apparatus. The filters were then blocked with skimmed milk, incubated with PRX002 or Syn1 antibody or the test GP antibodies of this disclosure at the indicated dilution. After extensive washing, a second anti-human or anti-Guinea pig IgG-HRP was used for detection of primary antibody binding profiles. A control with the secondary antibody only was also tested. Super Signal ECL (Pierce #34096) was used on the blots and the blots were then imaged on a BioRad imager (Chemidoc MP imaging system/BioRad imagelab software). The exposure time and the dynamic range are indicated on FIGS. 16A-16H. A human brain homogenate from a DLB case was spotted on the membrane in this set of measurements.

d. Results

The affinity of guinae pig (GP) antibodies PD-021514 (α-Syn₈₅₋₁₄₀, wpi 08), PD-021522 (α-Syn₈₅₋₁₄₀, wpi 13), PD-100806 (α-Syn₁₂₆₋₁₃₅, wpi 09) from immunized GPs, PRX002 and the commercial antibodies Syn1 (clone 42) was compared for distinct α-Syn assemblies using a filter trap assay. The α-Syn assemblies used included fibrils, ribbons, fibrils 65, fibrils 91, fibrils 110, on fibrillar assembly pathway α-Syn oligomers (O550), dopamine stabilized (ODA) and glutaraldehyde stabilized (OGA) oligomers, along with a control monomeric α-Syn.

FIGS. 16A-16H show that the reference antibody, PRX002, recognizes with slightly better affinity for fibrillar α-Syn, when compared to monomeric α-Syn; whereas PD-100806 and PD-021514, both directed against an α-Syn₁₂₆₋₁₃₅ peptide construct of this disclosure, have a much higher affinity for fibrillar α-Syn compared to monomeric α-Syn indicating that both have a preferential binding to fibrillar α-Syn. The affinities of PRX002 toward oligomeric and fibrillar α-Syn were found to be similar. Syn1 monoclonal antibody bound to fibrillar α-Syn as well as oligomeric and monomeric α-Syn without much differentiating preference.

### Example 17

### Immunohistochemistry Study for Antibodies Derived from α-Syn Peptide Immunogen Constructs with Brain Sections of Patients with Parkinson's Disease (PD), Multiple Systems Atrophy (MSA) and Demensia with Lewy Bodies (DLB)

Antibodies obtained from immunization of guinea pigs with a representative α-Syn₁₂₆₋₁₃₅ peptide immunogen construct of this invention were used in an immunochemical study to characterize their ability to bind to α-Syn present in brain sections from patients with alpha-synucleinopathies. The study was conducted in collaboration with Prof. Roxana Carare. The ability of the antibodies to bind α-Syn present in brain sections obtained from PD, LBD, and MSA patients was assessed. Healthy tissues were included in the study as a negative control. NCL-L-ASYN, a commercially available monoclonal antibody used for the post-mortem diagnosis of alpha-synucleinopathies, was included as a positive control. This investigation provides evidence of positive immunoreactivity of antibodies directed against α-Syn₁₂₆₋₁₃₅ peptide immunogen construct on tissue sections from human PD, LBD, and MSA patient brains. Binding was specifically seen in patient brains with synucleopathies but not in non-patient brains with the binding being more pronounced with the test antibodies than with the commercial diagnostic antibody.

### Methods and Materials

a. Description of Reagents Used and their Suppliers

Antibodies obtained from immunization in guinea pigs with a representative α-Syn₁₂₆₋₁₃₅ peptide immunogen construct were used at 1:100 dilution. PD062220-09-1-2-Syn; PD062205-09-1-2-Syn; PD100806-09-1-2-Syn were provided by United NeuroScience (UNS), NCL-L-ASYN (mouse monoclonal antibody used at 1:100 dilution) was provided by Leica Biosystems, HuD(E-I) (Mouse monoclonal antibody at 1:100 dilution) was provided by Santa Cruz Biotechnology, Olig2 (Rabbit antibodies at 1:100 dilution) was provided by Millipore, Alexa Flour 594 (Goat-anti-guinea pig at 1:200 dilution), Alexa Flour 488 (Goat-anti-mouse at 1:200), and Alexa Flour 488 (Goat-rabbit at 1:200 dilution) were provided by Molecular Probes life technologies.

b. Human Brain Tissue

Sections of μm thickness were obtained from the UCL brain bank were used in this study. All samples were collected and prepared in accordance with the National Research Ethics Service approved protocols.

Tissue was obtained from subjects (Table 15) with primary α-Syn pathology including multiple systems atrophy (MSA; n=3), Dementia with Lewy bodies (DLB; n=3) and Parkinson's disease (PD; n=3). Subjects were diagnosed post-mortem according to published criteria**.

c. Immunohistochemistry of Human Subjects of Synucleinopathies

Immunohistochemistry (IHC) on human subjects of three different synucleinopathies (MSA, DLB, and PD) was conducted in order to quantitatively compare the specificity for α-Syn aggregates of the three antibodies manufactured by United Neuroscience (UNS). The specificity of the UNS antibodies (PD062220, PD062205, and PD100806) for α-Syn aggregates was compared to the specificity of a commercially available diagnostic antibody (NCL-L-ASYN). Antibody specificity was analysed in the following four brain regions in each patient subject and disease type (1) Putamen, Internal Capsule, and Insula Cortex; (2) MidBrain: Substantia Nigra; (3) Temporal Cortex: Cortical Grey Matter; and (4) Cerebellum: Subcortical White Matter; Cerebellar White Matter.

These brain regions are known to be affected by α-Syn aggregation in varying degrees and at various stages of the disease progression in each disease type. Generally the basal ganglia and midbrain are affected early in DLB, PD, and MSA and also have the highest aggregate burden. The temporal cortex and cerebellum are affected at later stages of the disease with very little cerebellar aggregates present in PD and DLB. Negative controls (using no primary antibody) were run alongside each IHC protocol to confirm the absence of non-specific binding of the secondary antibody. Paraffin embedded slides were dewaxed in a 60° C. oven for 15-20 minutes and then immersed in Xylene I & II for 5 mins each. The tissue was rehydrated in 4 dilutions of IMS from 100% to 50% for 5 min each. The tissue was washed 3 times for 5 mins in 1×PBS and subsequently incubated for 3 mins in 100% formic acid for antigen retrieval. The tissue was washed thoroughly with 1×PBS before quenching endogenous peroxidase activity with 3% H₂O₂ for 10 min. The tissue was allowed to cool and washed a further 3 times in 1×PBS (5 min each) before microwaving in citrate buffer (15 mM tris sodium citrate, TWEEN, pH6) at medium heat for 25 min in order to ensure equal microwaving per run, 3 racks of slides in 3 containers were included each time. Slides were allowed to cool and were washed three times in 1×PBS (5 mins) prior to blocking non-specific binding sites with 15% normal goat serum. The tissue was incubated in primary antibody (1:100 in 0.1% TBS/t) overnight at 4° C. The tissue was washed 3×5 min in 1×PBS and incubated for 1 hr (RT) in a biotinylated secondary antibody. ABC solution was prepared 30 min prior to its application. After washing the tissue 3×5 min 1×PBS it was incubated in ABC for lhr at RT. VIP peroxidase substrate was prepared using ImmPACTVIP peroxidase kit as detailed in manufactures instructions. VIP peroxidase substrate was added for 7 min at RT and washed in dH₂O. Prior to mounting in DPX the tissue was dehydrated for 2 min each in IMS 50%, 70%, 95%, 100%, 100% and Xylenes I & II. For double immunofluorescent staining, the tissue was not quenched with 3% H₂O₂ prior to application of primary antibody. After application of the first primary and equivalent secondary antibodies the tissue was blocked with 15% normal goat serum for 30 min and incubated with the second primary and secondary antibody as described previously. After the final application of fluorescently tagged secondary antibodies, the tissue was incubated in 1% Sudan Black for 5 min to quench autofluorescence, washed in 0.1% TBS/T, and immediately mounted in mowiol cituflour. Fluorescently stained tissue was stored at 4° C. until imaged.

d. Image Analysis and Statistics

Slides were scanned for analysis at ×20 objective using either an Olympus VS110 high throughput Virtual Microscopy System or Olympus dot Slide Virtual Microscopy System. Thirty images (each 500 μm²) were captured from the scanned image using Olympus VS software from equivalent areas of each region from each subject (see FIGS. 17A-17D, 18A-18D, 19A-19C, 20A-20E, 21A-21F, 22A-22C, 24A-24D and 25A-25D). This allowed analysis of a total area of 7.5 mm² in each brain region. ImageJ version Fiji windows-64 software was used for the quantitative analysis of α-Syn immunoreactivity of each image.

For analysis of the total amount of α-Syn detected by each antibody, immunoreactivities were reported as a percentage of the total area of the image. The threshold applied for the selection of α-Syn positive immunoreactivity was adjusted for each brain region analysed in order to account for differences in background staining that could affect the results. The average percentage area covered by α-Syn positive aggregates was calculated for each antibody and brain region analysed.

For analysis of the relative specificity of each antibody for LBs or LNs, Fiji software was used to quantify the immunoreactivity of LBs based on parameters of size and circularity to distinguish them from LNs (see FIGS. 24A-24D, 25A-25D, and 26A-26B). Brain regions with distinct morphology of LBs and LNs were selected for this analysis to avoid false positives and included the insula cortex of the basal ganglia and cortical grey matter of the temporal cortex. LB immunoreactivity was expressed as a percentage of the total α-Syn immunoreactivity.

Statistical analysis was conducted using GraphPad Prism v7.01 software and are reported as mean+SD (unless otherwise specified). Results were analysed with a One-Way Analysis of Variance (ANOVA) followed by post hoc analysis with Dunnett corrections, where applicable. Differences were considered as significant when p<0.05 (*). Numbers (n) refer to the number of subjects used for each experiment.

Qualitative analysis of the location of α-Syn within neurones or glia was achieved by double-immunofluorescence staining as described previously. Slides were viewed with a Leica SP8 laser scanning confocal microscope. Maximal projections overlay images were obtained at ×40 objective in series. These images comprised a series of z-slide images stacked together with both color channels overlaid to show their relative positions.

e. α-Syn₁₂₆₋₁₃₅ Antibodies Detected a Different Pattern of α-Syn Aggregates, Compared to NCL-L-ASYN

The cell type and subcellular localisation of α-Syn aggregates vary between the different synucleinopathies. MSA is characterised by glial cytoplasmic inclusions (GCI), whereas in DLB and PD α-Syn aggregation occurs within neurone cell bodies (LBs) and axonal processes (LNs). Analysis of the percentage area stained enabled the quantification of the total α-Syn aggregates detected by each antibody. However, this did not take into account differences in the type or subcellular location of the aggregates detected. The distinct pattern of α-Syn aggregates within cell bodies and neurites in cases of PD and DLB enabled the relative sensitivity of UNS antibodies of this disclosure to these different types of α-Syn aggregates to be quantified.

In order to investigate this, the proportion of aggregates detected within cell bodies was estimated for each antibody in cases of DLB and PD. Using FIJI software, aggregates within cell bodies were selected based on their size and circularity. The average percentage area of cell-body aggregates was then calculated as a proportion of the total α-Syn detected and the results are shown in FIGS. 24A-24D and 25A-25D. The difference in the percentage area of total and cell-body α-Syn was attributed to axonal aggregates of α-Syn (LNs) based on qualitative analysis of the tissue. A decrease in the proportion of cell body α-Syn detection reciprocates an increase in LN detection. This analysis was conducted in the grey matter of the temporal cortex and insula cortex because these regions exhibited both LB and LN like pathology. LNs were very sparse and spread unevenly across the putamen and capsule and hence these regions of the basal ganglia were not selected for this analysis. A similar correlation was observed in the substantia nigra of the midbrain (FIGS. 26A and 26B) with UNS antibodies of this disclosure detecting higher levels of LNs compared to NCL-L-ASYN in DLB and PD. However, due to the complex morphology of the LNs and LBs it was not possible to reliably distinguish and quantify these with the same method.

The results in FIGS. 24A-24D show that, of the total α-Syn detected by each antibody, the proportion of aggregates detected within cell-bodies was decreased with UNS antibodies compared to NCL-L-ASYN. This means that the ratio of cell body inclusions to LNs was reduced, and a higher proportion of LNs was detected with UNS antibodies. Of the UNS antibodies, PD062205 was consistent between DLB and PD in detecting high proportions of LNs in the insula cortex (FIGS. 17A-17D and 18A-18D). In contrast, all the α-Syn₁₂₆₋₁₃₅ antibodies detected higher proportions of cell-body aggregates compared to NCL-L-ASYN in the temporal cortex grey matter of DLB and PD cases (FIG. 25A-25B).

f. Aggregation of α-Syn is Cell Type Specific

α-Syn containing aggregates are the characteristic pathogenic hallmark of the synucleinopathies including MSA, DLB, and PD. While α-Syn aggregation is the primary causative protein in synucleinopathies, the pattern of aggregation and cell-types that are susceptible to aggregate formation differ between specific disease sub-types. Clinical characterisation of MSA, DLB, and PD has described the accumulation of α-Syn within the cell bodies and neritic processes of neurones in both DLB and PD but in MSA it is found mainly within glia cells and oligodendrocytes.

In order to establish the selectivity of α-Syn₁₂₆₋₁₃₅ antibodies for cell-specific α-Syn aggregates, double-immunofluorescence was performed using PD062205 and markers for either neurones (HuD) or oligodendrocytes (Olig2).

The results in FIG. 27A-27C show that α-Syn, detected by PD062205, co-localizes within neuronal cell bodies in the basal ganglia and midbrain (regions of high pathology) in PD and DLB, but not MSA. Using markers for oligodendrocytes (Olig2), FIGS. 28A-28C show that in MSA, but not PD or DL, α-Syn aggregates within glia cells. These results demonstrate that the α-Syn₁₂₆₋₁₃₅ antibodies are consistent with clinical characterization of these synucleinopathies and confirm the specificity of these antibodies for pathological aggregates of α-Syn.

### Results

a. Quantitative Analysis of Antibodies Derived from Immunization in Guinea Pigs with a Representative α-Syn₁₂₆₋₁₃₅ Peptide Immunogen Construct for Immunotherapy

In order to investigate the use of novel anti-α-Syn antibodies for immunotherapy, quantitative analysis of the relative specificity of each antibody for α-Syn was performed by immunohistochemistry (IHC) in human cases of three synucleinopathies (MSA, DLB, and PD).

b. Antibodies Derived from Immunization in Guinea Pigs with a Representative α-Syn₁₂₆₋₁₃₅ Peptide Immunogen Construct is More Sensitive than Commercially Used Diagnostic Antibodies at Binding to α-Syn Aggregates

In order to investigate the relative antigenicity of the disclosed α-Syn₁₂₆₋₁₃₅ antibodies, the α-Syn load detected with each antibody was compared to a commercially available diagnostic antibody for synucleinopathies (NCL-L-ASYN). By first examining the overall pattern of results shown in FIGS. 17A-D to 22A-22C, it can be seen that there is a notable increase in the average percentage area of α-Syn detected with α-Syn₁₂₆₋₁₃₅ antibodies compared to NCL-L-ASYN. This trend is consistent across each brain region and disease type and suggests that the disclosed α-Syn₁₂₆₋₁₃₅ antibodies are more sensitive, or selective, at binding to aggregated α-Syn than NCL-L-ASYN. Although the sample size was relatively small in this study (n=3), a clear trend in the data can still be seen. The specificity of the disclosed α-Syn₁₂₆₋₁₃₅ antibodies for α-Syn was confirmed in the same brain regions from non-diseased control patient brains. These results, which are shown in FIGS. 23A-23B, shows the absence of any immune-positive staining with each antibody including NCL-L-ASYN. These data indicate that the disclosed α-Syn₁₂₆₋₁₃₅ antibodies are specific for pathological forms of α-Syn.

c. The Higher Level of α-Syn Detected Using α-Syn₁₂₆₋₁₃₅ Antibodies is Indicative of their Improved Sensitivity and Specificity when Compared to Commercial Antibodies

The α-Syn₁₂₆₋₁₃₅ antibodies of the present disclosure detect a larger amount of α-Syn when compared to NCL-L-ASYN, which indicates that the disclosed antibodies are more favorable for use in immunotherapy to facilitate clearance of these α-Syn aggregates.

The first step in selecting an appropriate antibody for use as an immunotherapy reagent is to establish the selectivity of the antibodies for the target antigen (α-Syn) in human brain tissue with primary α-Syn pathology. The different synucleinopathies vary in the mechanisms and neuroanatomical pattern of α-Syn aggregation as well as the vulnerability of specific cell types to aggregation.

It is important to assess the selectivity of the α-Syn₁₂₆₋₁₃₅ antibodies for α-Syn in different synucleinopathies with distinct neuropathology in order to investigate the use of a reagent as an immunotherapy for synucleinopathies in general. Clinically confirmed cases of PD, DLB, and MSA were selected for this purpose. PD and DLB are the second most common forms of dementia and are mainly caused by accumulation of α-Syn within neurons (LB and LN). In contrast to PD, amyloid-beta and tau pathologies are known to contribute to neurodegeneration in DLB2. A different pattern of α-Syn aggregation is seen in MSA where aggregates are mainly formed within glial cells rather than neurones (FIGS. 27A-27C and 28A-28B). In addition, the progression of α-Syn pathology varies between disease types with the midbrain and basal ganglia being common regions of early pathology. Examining the antigenicity of each antibody in brain regions affected at varying stages of the disease will provide insight as to which antibody may be more effective for treating early stages of the disease.

d. The α-Syn₁₂₆₋₁₃₅ Antibodies (PD062220, PD062205, and PD100806) are Capable of Specifically Binding to Pathological Aggregates of α-Syn in Human Brain Tissue from PD, DLB, and MSA (FIGS. 17A-D to 22A-22C) without Detecting any Synuclein Pathology in Healthy Controls (FIGS. 23A-23B).

Detection of α-Syn by the disclosed α-Syn₁₂₆₋₁₃₅ antibodies was achieved with the same cell-type specificity that has been described in clinical neuropathology (FIGS. 27A-27B and 28A-28B). Importantly, the disclosed α-Syn₁₂₆₋₁₃₅ antibodies did not demonstrate equal antigenicity for all forms of human α-Syn.

The specificity of PD062205 and PD100806 was further verified in each antibody's ability to detect a greater proportion of LNs than NCL-L-ASYN in the Basal Ganglia (FIGS. 24A-24D). This was also observed visually in the midbrain (FIG. 26A-26B). Taken together, with the higher percentage area of α-Syn detected by PD062205 and PD100806, these results indicate that the additional α-Syn detected by the disclosed α-Syn₁₂₆₋₁₃₅ antibodies can be partially attributed to an increased specificity of these antibodies for LNs. These results are beneficial for immunotherapy because, in early stages of the disease, LNs are the predominant form of α-Syn aggregation in the basal ganglia. Other reagents for treating synucleinopathies that are under preclinical development, do not provide IHC detection of LNs. Thus, the disclosed peptide immunogen constructs and α-Syn₁₂₆₋₁₃₅ antibodies generated from the peptide immunogen constructs have unique properties and features compared to other commercially-available products.

The present study utilized IHC to analyze the sensitivity of α-Syn₁₂₆₋₁₃₅ antibodies elicted by the disclosed peptide immunogen constructs by measuring the average amount of α-Syn aggregates in affected brain regions. The present study, which quantified the average percentage area of α-Syn in brain samples, demonstrates that the disclosed α-Syn₁₂₆₋₁₃₅ antibodies were the very sensitive to α-Syn detection earlier in the disease progression of MSA, DLB, and PD compared to a commercially available antibody.

The higher sensitivity found in this study can be attributed to a greater specificity of the disclosed antibodies to LNs over the diagnostic antibody, NCL-L-ASYN. These results suggest that the disclosed α-Syn₁₂₆₋₁₃₅ antibodies are likely to be the most effective candidates for the investigation of antibody-aided clearance of α-Syn aggregates in synucleinopathies.

TABLE 1
Amino Acid Sequences of α-Syn and Fragments Thereof Employed in Serological Assays

| | SEQ ID | |
|---|---|---|
| Amino Acid positions | NO: | Sequence |
| α-Synuclein 1-140 | 1 | MDVFM KGLSK AKEGV VAAAE KTKQG VAEAA GKTKE GVLYV GSKTK EGVVH |
| | | GVATV AEKTK EQVTN VGGAV VTGVT AVAQK TVEGA GSIAA ATGFV KKDQL |
| | | GKNEE GAPQE GILED MPVDP DNEAY EMPSE EGYQD YEPEA |
| α-Synuclein 80-140 | 3 | KTVEG AGSIA AATGF VKKDQ LGKNE EGAPQ EGILE DMPVD PDNEA YEMPS |
| | | EEGYQ DYEPE A |
| α-Synuclein 85-140 | 4 | AGSIA AATGF VKKDQ LGKNE EGAPQ EGILE DMPVD PDNEA YEMPS EEGYQ |
| | | DYEPEA |
| α-Synuclein 91-140 | 5 | ATGFV KKDQL GKNEE GAPQE GILED MPVDP DNEAY EMPSE EGYQD YEPEA |
| α-Synuclein 101-140 | 6 | GKNEE GAPQE GILED MPVDP DNEAY EMPSE EGYQD YEPEA |
| α-Synuclein 111-140 | 7 | GILED MPVDP DNEAY EMPSE EGYQD YEPEA |
| α-Synuclein 121-140 | 8 | DNEAY EMPSE EGYQD YEPEA |
| α-Synuclein 126-140 | 9 | EMPSE EGYQD YEPEA |
| α-Synuclein 97-135 | 10 | KDQLG KNEEG APQEG ILEDM PVDPD NEAYE MPSEE GYQD |
| α-Synuclein 101-135 | 11 | GKNEE GAPQE GILED MPVDP DNEAY EMPSE EGYQD |
| α-Synuclein 111-135 | 12 | GILED MPVDP DNEAY EMPSE EGYQD |
| α-Synuclein 121-135 | 13 | DNEAY EMPSE EGYQD |
| α-Synuclein 123-135 | 14 | EAYEM PSEEG YQD |
| α-Synuclein 126-135 | 15 | EMPSE EGYQD |
| α-Synuclein 101-132 | 16 | GKNEE GAPQE GILED MPVDP DNEAY EMPSE EG |
| α-Synuclein 111-132 | 17 | GILED MPVDP DNEAY EMPSE EG |
| α-Synuclein 80-89 | 18 | KTVEG AGSIA |
| α-Synuclein 81-90 | 19 | TVEGA GSIAA |
| α-Synuclein 82-91 | 20 | VEGAG SIAAA |
| α-Synuclein 83-92 | 21 | EGAGS IAAAT |
| α-Synuclein 84-93 | 22 | GAGSI AAATG |
| α-Synuclein 85-94 | 23 | AGSIA AATGF |
| α-Synuclein 86-95 | 24 | GSIAA ATGFV |
| α-Synuclein 87-96 | 25 | SIAAA TGFVK |
| α-Synuclein 88-97 | 26 | IAAAT GFVKK |
| α-Synuclein 89-98 | 27 | AAATG FVKKD |
| α-Synuclein 90-99 | 28 | AATGF VKKDQ |
| α-Synuclein 91-100 | 29 | ATGFV KKDQL |
| α-Synuclein 92-101 | 30 | TGFVK KDQLG |
| α-Synuclein 93-102 | 31 | GFVKK DQLGK |
| α-Synuclein 94-103 | 32 | FVKKD QLGKN |
| α-Synuclein 95-104 | 33 | VKKDQ LGKNE |
| α-Synuclein 96-105 | 34 | KKDQL GKNEE |
| α-Synuclein 97-106 | 35 | KDQLG KNEEG |
| α-Synuclein 98-107 | 36 | DQLGK NEEGA |
| α-Synuclein 99-108 | 37 | QLGKN EEGAP |
| α-Synuclein 100-109 | 38 | LGKNE EGAPQ |
| α-Synuclein 101-110 | 39 | GKNEE GAPQE |
| α-Synuclein 102-111 | 40 | KNEEG APQEG |
| α-Synuclein 103-112 | 41 | NEEGA PQEGI |
| α-Synuclein 104-113 | 42 | EEGAP QEGIL |
| α-Synuclein 105-114 | 43 | EGAPQ EGILE |
| α-Synuclein 106-115 | 44 | GAPQE GILED |
| α-Synuclein 107-116 | 45 | APQEG ILEDM |
| α-Synuclein 108-117 | 46 | PQEGI LEDMP |
| α-Synuclein 109-118 | 47 | QEGIL EDMPV |
| α-Synuclein 110-119 | 48 | EGILE DMPVD |
| α-Synuclein 111-120 | 49 | GILED MPVDP |
| α-Synuclein 112-121 | 50 | ILEDM PVDPD |
| α-Synuclein 113-122 | 51 | LEDMP VDPDN |
| α-Synuclein 114-123 | 52 | EDMPV DPDNE |
| α-Synuclein 115-124 | 53 | DMPVD PDNEA |
| α-Synuclein 116-125 | 54 | MPVDP DNEAY |
| α-Synuclein 117-126 | 55 | PVDPD NEAYE |
| α-Synuclein 118-127 | 56 | VDPDN EAYEM |
| α-Synuclein 119-128 | 57 | DPDNE AYEMP |
| α-Synuclein 120-129 | 58 | PDNEA YEMPS |
| α-Synuclein 121-130 | 59 | DNEAY EMPSE |
| α-Synuclein 122-131 | 60 | NEAYE MPSEE |
| α-Synuclein 123-132 | 61 | EAYEM PSEEG |
| α-Synuclein 124-133 | 62 | AYEMP SEEGY |
| α-Synuclein 125-134 | 63 | YEMPS EEGYQ |
| α-Synuclein 126-135 | 64 | EMPSE EGYQD |
| α-Synuclein 127-136 | 65 | MPSEE GYQDY |
| α-Synuclein 128-137 | 66 | PSEEG YQDYE |
| α-Synuclein 129-138 | 67 | SEEGY QDYEP |
| α-Synuclein 130-139 | 68 | EEGYQ DYEPE |
| α-Synuclein 131-140 | 69 | EGYQD YEPEA |

TABLE 2
Amino Acid Sequences of Pathogen Protein Derived Th Epitopes
Including Idealized Artificial Th Epitopes for Employment in the
Design of α-Syn Peptide Immunogen Constructs

| | SEQ ID | |
|---|---|---|
| Description | NO: | Sequence |
| Clostridium tetani1 Th | 70 | KKQYIKANSKFIGITEL |
| MvF1 Th | 71 | LSEIKGVIVHRLEGV |
| Bordetella pertussis Th | 72 | GAYARCPNGTRALTVAELRGNAEL |
| Clostridium tetani2 Th | 73 | WVRDIIDDFTNESSQKT |
| Diphtheria Th | 74 | DSETADNLEKTVAALSILPGHGC |
| Plasmodium falciparum Th | 75 | DHEKKHAKMEKASSVFNVVNS |
| Schistosoma mansoni Th | 76 | KWFKTNAPNGVDEKHRH |
| Cholera Toxin Th | 77 | ALNIWDRFDVFCTLGATTGYLKGNS |
| MvF2 Th | 78 | ISEIKGVIVHKIEGI |
| KKKMvF3 Th | 79 | KKKISISEIKGVIVHKIEGILF |
| | | T RT TR T |
| HBsAg1 Th | 80 | KKKLFLLTKLLTLPQSLD |
| | | RRRIKII RII I L IR |
| | | VRVV VV V I V |
| | | F FF FF F V F |
| | | F |
| MvF4 Th (UBITh ®3) | 81 | ISISEIKGVIVHKIETILF |
| | | T RT TR |
| HBsAg2 Th | 82 | KKKIITITRIITIPQSLD |
| | | FFLL L ITTI |
| MvF5 Th (UBITh ®1) | 83 | ISITEIKGVIVHRIETILF |
| HBsAg3 Th (UBITh ®2) | 84 | KKKIITITRIITIITTID |
| Influenza MP1_1 Th | 85 | FVFTLTVPSER |
| Influenza MP1_2 Th | 86 | SGPLKAEIAQRLEDV |
| Influenza NSP1 Th | 87 | DRLRRDQKS |
| EBV BHRF1 Th | 88 | AGLTLSLLVICSYLFISRG |
| Clostridium tetani TT1 Th | 89 | QYIKANSKFIGITEL |
| EBV EBNA-1 Th | 90 | PGPLRESIVCYFMVFLQTHI |
| Clostridium tetani TT2 Th | 91 | FNNFTVSFWLRVPKVSASHLE |
| Clostridium tetani TT3 Th | 92 | KFIIKRYTPNNEIDSF |
| Clostridium tetani TT4 Th | 93 | VSIDKFRIFCKALNPK |
| EBV CP Th | 94 | VPGLYSPCRAFFNKEELL |
| HCMVIE1 Th | 95 | DKREMWMACIKELH |
| EBV GP340 Th | 96 | TGHGARTSTEPTTDY |
| EBV BPLF1 Th | 97 | KELKRQYEKKLRQ |
| EBV EBNA-2 Th | 98 | TVFYNIPPMPL |

TABLE 3
Amino Acid Sequences of a-Syn Peptide Immunogen Constructs

| | Seq | |
|---|---|---|
| | ID | |
| Peptide Description | NO: | Sequence |
| UBITh3-ϵK-KKK-α-Synuclein 126-140 | 99 | UBITh3-ϵk-kkk-EMPSEEGYQDYEPEA |
| UBITh3-ϵK-KKK-α-Synuclein 121-140 | 100 | UBITh3-ϵk-kkk-DNEAYEMPSEEGYQDYEPEA |
| UBITh3-ϵK-KKK-α-Synuclein 111-140 | 101 | UBITh3-ϵk-kkk-GILEDMPVDPDNEAYEMPSEEGYQDYEPEA |
| UBITh3-ϵK-KKK-α-Synuclein 101-140 | 102 | UBITh3-ϵk-kkk-GKNEEGAPQEGILEDMPVDPDNEAYEMPSEEGYQDYEPEA |
| UBITh1-ϵK-KKK-α-Synuclein 101-140 | 103 | UBITh1-ϵk-kkk-GKNEEGAPQEGILEDMPVDPDNEAYEMPSEEGYQDYEPEA |
| UBITh2-ϵK-KKK-α-Synuclein 101-140 | 104 | UBITh2-ϵk-kkk-GKNEEGAPQEGILEDMPVDPDNEAYEMPSEEGYQDYEPEA |
| UBITh3-ϵK-KKK-α-Synuclein 91-140 | 105 | UBITh3-ϵk-kkk- |
| | | ATGFVKKDQLGKNEEGAPQEGILEDMPVDPDNEAYEMPSEEGYQDYEPEA |
| UBITh3-ϵK-KKK-α-Synuclein 85-140 | 106 | UBITh3-ϵk-kkk- |
| | | AGSIAAATGFVKKDQLGKNEEGAPQEGILEDMPVDPDNEAYEMPSEEGYQDYEPEA |
| UBITh1-ϵK-KKK-α-Synuclein 121-135 | 107 | UBITh1-ϵk-kkk-DNEAYEMPSEEGYQD |
| UBITh1-ϵK-KKK-α-Synuclein 111-135 | 108 | UBITh1-ϵk-kkk-GILEDMPVDPDNEAYEMPSEEGYQD |
| UBITh1-ϵK-KKK-α-Synuclein 101-135 | 109 | UBITh1-ϵk-kkk-GKNEEGAPQEGILEDMPVDPDNEAYEMPSEEGYQD |
| UBITh1-ϵK-KKK-α-Synuclein 97-135 | 110 | UBITh1-ϵk-kkk-KDQLGKNEEGAPQEGILEDMPVDPDNEAYEMPSEEGYQD |
| UBITh1-ϵK-KKK-α-Synuclein 123-135 | 111 | UBITh1-ϵk-kkk-EAYEMPSEEGYQD |
| UBITh1-ϵK-KKK-α-Synuclein 126-135 | 112 | UBITh1-ϵk-kkk-EMPSEEGYQD |
| UBITh1-ϵK-KKK-α-Synuclein 111-132 | 113 | UBITh1-ϵk-kkk-GILEDMPVDPDNEAYEMPSEEG |
| UBITh1-ϵK-KKK-α-Synuclein 101-132 | 114 | UBITh1-ϵk-kkk-GKNEEGAPQEGILEDMPVDPDNEAYEMPSEEG |
| UBITh1-ϵK-KKK-Mouse counterpart | 115 | UBITh1-ϵk-kkk-GILEDMPVDPGSEAYEMPSEEG |
| α-Synuclein 111-132 | | |
| UBITh3-ϵK-KKK-α-Synuclein 126-135 | 116 | UBITh3-ϵk-kkk-EMPSEEGYQD |
| UBITh3-ϵK-KKK-α-Synuclein 111-132 | 117 | UBITh3-ϵk-kkk-GILEDMPVDPDNEAYEMPSEEG |
| UBITh1-ϵK-α-Synuclein 126-135 | 118 | UBITh1-ϵk-EMPSEEGYQD |
| UBITh1-ϵK-α-Synuclein 111-132 | 119 | UBITh1-ϵk-GILEDMPVDPDNEAYEMPSEEG |
| UBITh2-ϵK-α-Synuclein 126-135 | 120 | UBITh2-ϵk-EMPSEEGYQD |
| UBITh2-ϵK-α-Synuclein 111-132 | 121 | UBITh2-ϵk-GILEDMPVDPDNEAYEMPSEEG |
| Clostridium tetani1 Th-ϵK-α-Syn 111-132 | 122 | KKQYIKANSKFIGITEL-ϵk-GILEDMPVDPDNEAYEMPSEEG |
| MvF1 Th-ϵK-α-Synuclein 111-132 | 123 | LSEIKGVIVHRLEGV-ϵk-GILEDMPVDPDNEAYEMPSEEG |
| Bordetella pertussis Th-ϵK-α-Syn 111-132 | 124 | GAYARCPNGTRALTVAELRGNAEL-ϵk-GILEDMPVDPDNEAYEMPSEEG |
| Clostridium tetani2 Th-ϵK-α-Syn 111-132 | 125 | WVRDIIDDFTNESSQKT-ϵk-GILEDMPVDPDNEAYEMPSEEG |
| Diphtheria Th-ϵK-α-Syn 111-132 | 126 | DSETADNLEKTVAALSILPGHGC-ϵk-GILEDMPVDPDNEAYEMPSEEG |
| Plasmodium falciparum Th-ϵK-α-Syn 111-132 | 127 | DHEKKHAKMEKASSVFNVVNS-ϵk-GILEDMPVDPDNEAYEMPSEEG |
| Schistosoma mansoni Th-ϵK-α-Syn 111-132 | 128 | KWFKTNAPNGVDEKHRH-ϵk-GILEDMPVDPDNEAYEMPSEEG |
| Cholera Toxin Th-ϵK-α-Syn 111-132 | 129 | ALNIWDRFDVFCTLGATTGYLKGNS-ϵk-GILEDMPVDPDNEAYEMPSEEG |
| MvF2 Th-ϵK-α-Syn 111-132 | 130 | ISEIKGVIVHKIEGI-ϵk-GILEDMPVDPDNEAYEMPSEEG |
| KKKMvF3 Th-ϵK-α-Syn 111-132 | 131 | KKKISISEIKGVIVHKIEGILF-ϵk-GILEDMPVDPDNEAYEMPSEEG |
| | | T RT TR T |
| HBsAg1 Th-ϵK-α-Syn 111-132 | 132 | KKKLFLLTKLLTLPQSLD-ϵk-GILEDMPVDPDNEAYEMPSEEG |
| | | RRRIKII RII I L IR |
| | | VRVV VV V I V |
| | | F FF FF F V F |
| | | F |
| HBsAg2 Th-ϵK-α-Syn 111-132 | 133 | KKKIITITRIITIPQSLD-ϵk-GILEDMPVDPDNEAYEMPSEEG |
| | | FFLL L ITTI |
| Influenza MP1_1 Th-ϵK-α-Syn 111-132 | 134 | FVFTLTVPSER-ϵk-GILEDMPVDPDNEAYEMPSEEG |
| Influenza MP1_2 Th-ϵK-α-Syn 111-132 | 135 | SGPLKAEIAQRLEDV-ϵk-GILEDMPVDPDNEAYEMPSEEG |
| Influenza NSP1 Th-ϵK-α-Syn 111-132 | 136 | DRLRRDQKS-ϵk-GILEDMPVDPDNEAYEMPSEEG |
| EBV BHRF1 Th-ϵK-α-Syn 111-132 | 137 | AGLTLSLLVICSYLFISRG-ϵk-GILEDMPVDPDNEAYEMPSEEG |
| Clostridium tetani TT1 Th-ϵK-α-Syn 111-132 | 138 | QYIKANSKFIGITEL-ϵk-GILEDMPVDPDNEAYEMPSEEG |
| EBV EBNA-1 Th-ϵK-α-Syn 111-132 | 139 | PGPLRESIVCYFMVFLQTHI-ϵk-GILEDMPVDPDNEAYEMPSEEG |
| Clostridium tetani TT2 Th-ϵK-α-Syn 111-132 | 140 | FNNFTVSFWLRVPKVSASHLE-ϵk-GILEDMPVDPDNEAYEMPSEEG |
| Clostridium tetani TT3 Th-ϵK-α-Syn 111-132 | 141 | KFIIKRYTPNNEIDSF-ϵk-GILEDMPVDPDNEAYEMPSEEG |
| Clostridium tetani TT4 Th-ϵK-α-Syn 111-132 | 142 | VSIDKFRIFCKALNPK-ϵk-GILEDMPVDPDNEAYEMPSEEG |
| EBV CP Th-ϵK-α-Syn 111-132 | 143 | VPGLYSPCRAFFNKEELL-ϵk-GILEDMPVDPDNEAYEMPSEEG |
| HCMVIE1 Th-ϵK-α-Syn 111-132 | 144 | DKREMWMACIKELH-ϵk-GILEDMPVDPDNEAYEMPSEEG |
| EBV GP340 Th-ϵK-α-Syn 111-132 | 145 | TGHGARTSTEPTTDY-ϵk-GILEDMPVDPDNEAYEMPSEEG |
| EBV BPLF1 Th-ϵK-α-Syn 111-132 | 146 | KELKRQYEKKLRQ-ϵk-GILEDMPVDPDNEAYEMPSEEG |
| EBV EBNA-2 Th-ϵK-α-Syn 111-132 | 147 | TVFYNIPPMPL-ϵk-GILEDMPVDPDNEAYEMPSEEG |

TABLE 4
Immunogenicity Assessment in Guinea Pigs of C-terminal α-Syn
Peptide Fragments for Identification of Autologous Th Epitopes

| | Seq | | α-Syn _{(A85-A140)} (SEQ ID NO: 4) | | | |
|---|---|---|---|---|---|---|
| Peptide | ID | Animal | ELISA Logio Titer | | | |
| Description | NO: | ID | 0 wpi | 3 wpi | 6 wpi | 8 wpi |
| α-synuclein | 9 | 5413 | 0.075 | 0.000 | 0.000 | 0.000 |
| (E126-A140) | | 5414 | 0.086 | 0.000 | 0.000 | 0.000 |
| | | 5415 | 0.079 | 0.000 | 0.000 | 0.000 |
| | | Avg | 0.080 | 0.000 | 0.000 | 0.000 |
| α-synuclein | 8 | 5416 | 0.056 | 0.000 | 0.000 | 0.000 |
| (D121-A140) | | 5417 | 0.091 | 0.000 | 0.000 | 0.000 |
| | | 5418 | 0.066 | 0.000 | 0.000 | 0.000 |
| | | Avg | 0.071 | 0.000 | 0.000 | 0.000 |
| α-synuclein | 7 | 5419 | 0.060 | 0.000 | 0.000 | 0.000 |
| (G111-A140) | | 5420 | 0.089 | 0.000 | 0.000 | 1.026 |
| | | 5421 | 0.092 | 0.139 | 0.000 | 0.000 |
| | | Avg | 0.081 | 0.046 | 0.000 | 0.342 |
| α-synuclein | 6 | 5422 | 0.084 | 0.000 | 1.997 | 3.096 |
| (G101-A140) | | 5423 | 0.072 | 0.000 | 0.000 | 0.000 |
| | | 5424 | 0.077 | 0.000 | 0.000 | 0.000 |
| | | Avg | 0.078 | 0.000 | 0.666 | 1.032 |
| α-synuclein | 5 | 5425 | 0.082 | 0.000 | 0.000 | 0.000 |
| (A91-A140) | | 5426 | 0.079 | 0.294 | 3.007 | 2.765 |
| | | 5427 | 0.093 | 0.000 | 2.840 | 3.355 |
| | | Avg | 0.084 | 0.098 | 1.949 | 2.040 |
| α-synuclein | 4 | 5428 | 0.082 | 3.059 | 3.628 | 4.349 |
| (A85-A140) | | 5429 | 0.082 | 0.000 | 0.000 | 0.000 |
| | | 5430 | 0.073 | 0.000 | 3.005 | 2.894 |
| | | Avg | 0.079 | 1.020 | 2.211 | 2.414 |

TABLE 5
Immunogenicity Ranking in Guinea Pigs of α-Syn Peptide Immunogen Constructs

| | | | | α-Syn _{(G101-A140)} (SEQ ID NO: 6) | | | |
|---|---|---|---|---|---|---|---|
| Group | α-synuclein peptide | Seq ID | Animal | ELISA Log₁₀ Titer | | | |
| # | immunogen construct | NO: | ID | 0 wpi | 3 wpi | 6 wpi | 8 wpi |
| 1 | UBITh1-εK-KKK- | 103 | 5431 | 0.167 | 4.740 | 4.938 | 4.912 |
| | α-synuclein (G101-A140) | | 5432 | 0.111 | 4.787 | 4.979 | 4.819 |
| | | | 5433 | 0.110 | 4.799 | 4.920 | 4.924 |
| | | | Avg | 0.129 | 4.775 | 4.946 | 4.885 |
| 2 | UBITh2-εK-KKK- | 104 | 5434 | 0.101 | 0.000 | 3.095 | 3.172 |
| | α-synuclein (G101-A140) | | 5435 | 0.100 | 2.743 | 4.439 | 4.052 |
| | | | 5436 | 0.097 | 0.967 | 1.790 | 1.952 |
| | | | Avg | 0.099 | 1.237 | 3.108 | 3.059 |

TABLE 6
Immunogenicity Assessment in Guinea Pigs of α-Syn Peptide Immunogen Constructs

| | | | | α-Syn _{(A91-A140)} | | | | | β-Syn ₍₁₀₃₋₁₃₄₎ | | | | |
|---|---|---|---|---|---|---|---|---|---|---|---|---|---|
| | | Seq | | (SEQ ID NO: 5) | | | | | (SEQ ID NO: 153) | | | | |
| Group | | ID | Animal | ELISA Log₁₀ Titer | | | | | ELISA Log₁₀ Titer | | | | |
| # | Immunogen | NO: | No. | 0 w | 3 w | 6 w | 8 w | 13 w | 0 w | 3 w | 6 w | 8 w | 13 w |
| 1 | UBITh3-εk-kkk- | 99 | 5334 | 0.1 | 5.1 | 5.3 | 6.6 | 5.0 | 0.1 | 2.9 | 4.4 | 4.9 | 4.7 |
| | α-synuclein (E126-A140) | | 5335 | 0.1 | 5.3 | 5.5 | 5.4 | 5.0 | 0.1 | 3.9 | 4.8 | 4.9 | 4.7 |
| | | | 5336 | 0.1 | 6.9 | 11.0 | 12.5 | 8.3 | 0.1 | 3.8 | 5.2 | 5.7 | 5.4 |
| | | | Avg | 0.1 | 5.8 | 7.3 | 8.2 | 6.1 | 0.1 | 3.5 | 4.8 | 5.2 | 4.9 |
| 2 | UBITh3-εk-kkk- | 100 | 5337 | 0.1 | 5.1 | 4.9 | 5.1 | 4.9 | 0.1 | 2.5 | 4.3 | 4.6 | 4.5 |
| | α-synuclein (D121-A140) | | 5338 | 0.2 | 4.5 | 4.6 | 4.7 | 4.4 | 0.1 | 1.3 | 3.3 | 3.8 | 3.3 |
| | | | 5339 | 0.1 | 4.7 | 4.9 | 5.1 | 4.7 | 0.1 | 2.0 | 4.4 | 4.6 | 4.3 |
| | | | Avg | 0.1 | 4.8 | 4.8 | 5.0 | 4.7 | 0.1 | 1.9 | 4.0 | 4.4 | 4.1 |
| 3 | UBITh3-εk-kkk- | 101 | 5340 | 0.2 | 5.1 | 5.0 | 5.1 | 4.6 | 0.1 | 2.2 | 3.9 | 4.4 | 3.5 |
| | α-synuclein (G111-A140) | | 5341 | 0.1 | 7.1 | 7.8 | 9.2 | 6.2 | 0.1 | 3.6 | 4.9 | 5.0 | 4.9 |
| | | | 5342 | 0.1 | 4.9 | 5.2 | 5.8 | 5.2 | 0.1 | 2.0 | 4.6 | 4.8 | 4.8 |
| | | | Avg | 0.1 | 5.7 | 6.0 | 6.7 | 5.4 | 0.1 | 2.6 | 4.5 | 4.7 | 4.4 |
| 4 | UBITh3-εk-kkk- | 102 | 5343 | 0.2 | 6.0 | 8.5 | 12.0 | 7.3 | 0.1 | 4.3 | 5.2 | >5.00 | 5.8 |
| | α-synuclein (G101-A140) | | 5344 | 0.3 | 6.6 | 5.7 | 6.0 | 5.3 | 0.1 | 4.0 | 4.7 | 4.8 | 4.6 |
| | | | 5345 | 0.2 | 5.9 | 6.2 | 9.4 | 5.9 | 0.1 | 4.0 | 5.0 | 5.5 | 5.2 |
| | | | Avg | 0.2 | 6.2 | 6.8 | 9.1 | 6.1 | 0.1 | 4.1 | 4.9 | 5.1 | 5.2 |
| 5 | UBITh3-εk-kkk- | 105 | 5362 | 0.2 | 5.5 | 6.6 | 8.0 | 5.5 | 0.1 | 3.6 | 4.8 | 5.0 | 4.8 |
| | α-synuclein (A91-A140) | | 5363 | 0.1 | 5.1 | 5.7 | 5.7 | 5.4 | 0.1 | 2.8 | 4.4 | 4.5 | 4.5 |
| | | | 5364 | 0.2 | 4.8 | 4.9 | 4.9 | 4.9 | 0.1 | 0.0 | 3.0 | 3.5 | 3.6 |
| | | | Avg | 0.1 | 5.2 | 5.7 | 6.2 | 5.3 | 0.1 | 2.1 | 4.1 | 4.3 | 4.3 |
| 6 | UBITh3-εk-kkk- | 106 | 5365 | 0.1 | 5.1 | 5.0 | 5.3 | 5.1 | 0.1 | 3.2 | 3.9 | 4.3 | 3.6 |
| | α-synuclein (A85-A140) | | 5366 | 0.2 | 5.4 | 4.9 | 4.9 | 4.8 | 0.1 | 3.2 | 3.2 | 3.1 | 3.1 |
| | | | 5367 | 0.1 | 5.1 | 5.3 | 5.3 | 5.3 | 0.1 | 1.1 | 4.7 | 4.7 | 4.6 |
| | | | Avg | 0.1 | 5.2 | 5.1 | 5.2 | 5.1 | 0.1 | 2.1 | 3.9 | 4.0 | 3.7 |

TABLE 7
Immunogenicity Assessment in Guinea Pigs of α-Syn Peptide Immunogen Constructs

| | | | | α-Syn (K97-D135) | | | | | β-Syn (103-134) | | | | |
|---|---|---|---|---|---|---|---|---|---|---|---|---|---|
| | | Seq | | (SEQ ID NO: 10) | | | | | (SEQ ID NO: 153) | | | | |
| Group | | ID | Animal | ELISA Log₁₀ Titer | | | | | ELISA Log₁₀ Titer | | | | |
| # | Immunogen | NO: | No | 0 w | 3 w | 6 w | 9 w | 12 w | 0 w | 3 w | 6 w | 9 w | 12 w |
| 1 | UBITh1-εk-kkk- | 110 | 5616 | 0.055 | 4.814 | 5.132 | 4.823 | 4.776 | 0.051 | 0.000 | 0.000 | 0.000 | 0.000 |
| | α-Synuclein (K97-0135) | | 5617 | 0.049 | 3.394 | 4.464 | 4.323 | 4.292 | 0.050 | 0.000 | 0.000 | 0.000 | 0.000 |
| | | | 5618 | 0.052 | 4.420 | 4.864 | 4.673 | 4.598 | 0.051 | 0.000 | 0.000 | 0.000 | 0.000 |
| | | | Avg. | 0.052 | 4.209 | 4.820 | 4.606 | 4.555 | 0.051 | 0.000 | 0.000 | 0.000 | 0.000 |
| 2 | UBITh1-εk-kkk- | 109 | 5613 | 0.056 | 4.738 | 4.882 | 4.848 | 4.855 | 0.056 | 0.000 | 0.000 | 0.000 | 0.000 |
| | α-Synuclein (G101-0135) | | 5614 | 0.052 | 4.391 | 4.708 | 4.565 | 4.674 | 0.053 | 0.000 | 0.000 | 0.000 | 0.000 |
| | | | 5615 | 0.058 | 4.789 | 5.050 | 4.956 | 4.904 | 0.055 | 0.000 | 0.000 | 0.000 | 0.000 |
| | | | Avg. | 0.055 | 4.639 | 4.880 | 4.790 | 4.811 | 0.055 | 0.000 | 0.000 | 0.000 | 0.000 |
| 3 | UBITh1-εk-kkk- | 114 | 5628 | 0.049 | 4.290 | 4.794 | 4.426 | 4.537 | 0.053 | 0.000 | 0.000 | 0.000 | 0.000 |
| | α-Synuclein (G101-G132) | | 5629 | 0.069 | 4.502 | 4.939 | 4.764 | 4.645 | 0.067 | 0.000 | 0.000 | 0.000 | 0.000 |
| | | | 5630 | 0.053 | 2.978 | 3.695 | 4.092 | 4.274 | 0.056 | 0.000 | 0.000 | 0.000 | 0.000 |
| | | | Avg. | 0.057 | 3.923 | 4.476 | 4.427 | 4.485 | 0.059 | 0.000 | 0.000 | 0.000 | 0.000 |
| 4 | UBITh1-εk-kkk- | 108 | 5545 | 0.051 | 4.941 | 4.919 | 4.842 | 4.735 | 0.069 | 0.000 | 0.000 | 0.000 | 0.000 |
| | α-Synuclein (G111-0135) | | 5546 | 0.056 | 3.229 | 4.866 | 4.912 | 4.843 | 0.063 | 0.000 | 0.000 | 0.000 | 0.000 |
| | | | 5547 | 0.053 | 5.075 | 5.237 | 5.033 | 4.954 | 0.065 | 0.000 | 0.000 | 0.000 | 0.000 |
| | | | Avg. | 0.053 | 4.415 | 5.007 | 4.929 | 4.844 | 0.066 | 0.000 | 0.000 | 0.000 | 0.000 |
| 5 | UBITh1-εk-kkk- | 113 | 5625 | 0.056 | 2.906 | 4.541 | 4.346 | 4.114 | 0.069 | 0.000 | 0.000 | 0.000 | 0.000 |
| | α-Synuclein (G111-G132) | | 5626 | 0.051 | 2.596 | 4.087 | 3.504 | 3.655 | 0.053 | 0.000 | 0.000 | 0.000 | 0.000 |
| | | | 5627 | 0.052 | 3.471 | 4.633 | 4.333 | 4.415 | 0.056 | 0.000 | 0.000 | 0.000 | 0.000 |
| | | | Avg. | 0.053 | 2.991 | 4.420 | 4.061 | 4.061 | 0.059 | 0.000 | 0.000 | 0.000 | 0.000 |
| 6 | UBITh1-εk-kkk- | 107 | 5542 | 0.067 | 3.042 | 4.214 | 4.121 | 3.989 | 0.062 | 0.000 | 0.000 | 0.000 | 0.000 |
| | α-Synuclein (0121-0135) | | 5543 | 0.054 | 4.733 | 4.948 | 4.832 | 4.862 | 0.062 | 0.000 | 0.000 | 0.000 | 0.000 |
| | | | 5544 | 0.060 | 2.943 | 4.306 | 4.249 | 4.222 | 0.065 | 0.000 | 0.000 | 0.000 | 0.000 |
| | | | Avg. | 0.060 | 3.573 | 4.489 | 4.401 | 4.358 | 0.063 | 0.000 | 0.000 | 0.000 | 0.000 |
| 7 | UBITh1-εk-kkk- | 111 | 5619 | 0.074 | 4.538 | 4.923 | 4.792 | 4.750 | 0.053 | 0.000 | 0.000 | 0.000 | 0.000 |
| | α-Synuclein (E123-0135) | | 5620 | 0.052 | 4.880 | 5.930 | 5.069 | 5.046 | 0.054 | 0.000 | 0.000 | 0.000 | 0.000 |
| | | | 5621 | 0.058 | 4.073 | 4.932 | 4.898 | 4.940 | 0.058 | 0.000 | 0.000 | 0.000 | 0.000 |
| | | | Avg. | 0.061 | 4.497 | 5.262 | 4.920 | 4.912 | 0.055 | 0.000 | 0.000 | 0.000 | 0.000 |
| 8 | UBITh1-εk-kkk- | 112 | 5622 | 0.051 | 4.820 | 5.156 | 5.015 | 5.018 | 0.055 | 0.000 | 0.000 | 0.000 | 0.000 |
| | α-Synuclein (E126-0135) | | 5623 | 0.054 | 4.190 | 5.035 | 4.990 | 4.958 | 0.058 | 0.000 | 0.000 | 0.000 | 0.000 |
| | | | 5624 | 0.048 | 4.906 | 6.747 | 5.630 | 5.602 | 0.063 | 0.000 | 0.000 | 0.000 | 0.000 |
| | | | Avg. | 0.051 | 4.639 | 5.646 | 5.212 | 5.193 | 0.059 | 0.000 | 0.000 | 0.000 | 0.000 |

TABLE 8
Immunogenicity Assessment in Guinea Pigs against the Th
Epitope Portion of the α-Syn Peptide Immunogen Constructs

| | | SEQ | | UBITh1 (SEQ ID NO: 83) | | | | |
|---|---|---|---|---|---|---|---|---|
| Group | | ID | Animal | ELISA Log₁₀ titer | | | | |
| # | Immunogen | NO: | ID | 0 w | 3 w | 6 w | 9 w | 12 w |
| 1 | UBITh1-εk-kkk-α-Synuclein | 110 | 5616 | 0.065 | 0.000 | 0.616 | 1.746 | 2.023 |
| | (K97-D135) | | 5617 | 0.052 | 0.000 | 0.000 | 0.000 | 0.000 |
| | | | 5618 | 0.058 | 0.000 | 0.000 | 0.000 | 0.000 |
| | | | Avg. | 0.058 | 0.000 | 0.205 | 0.582 | 0.674 |
| 2 | UBITh1-εk-kkk-α-Synuclein | 109 | 5613 | 0.057 | 0.000 | 0.000 | 0.000 | 0.000 |
| | (G101-D135) | | 5614 | 0.054 | 0.000 | 0.000 | 0.000 | 0.000 |
| | | | 5615 | 0.063 | 0.000 | 0.000 | 1.527 | 1.462 |
| | | | Avg. | 0.058 | 0.000 | 0.000 | 0.509 | 0.487 |
| 3 | UBITh1-εk-kkk-α-Synuclein | 114 | 5628 | 0.052 | 0.000 | 0.000 | 0.000 | 0.000 |
| | (G101-G132) | | 5629 | 0.062 | 0.000 | 0.000 | 0.000 | 0.000 |
| | | | 5630 | 0.058 | 0.000 | 0.000 | 0.000 | 0.000 |
| | | | Avg. | 0.057 | 0.000 | 0.000 | 0.000 | 0.000 |
| 4 | UBITh1-εk-kkk-α-Synuclein | 108 | 5545 | 0.065 | 0.000 | 0.000 | 0.000 | 0.000 |
| | (G111-D135) | | 5546 | 0.069 | 0.000 | 0.000 | 0.000 | 0.000 |
| | | | 5547 | 0.060 | 0.000 | 0.095 | 1.105 | 1.175 |
| | | | Avg. | 0.065 | 0.000 | 0.032 | 0.368 | 0.392 |
| 5 | UBITh1-εk-kkk-α-Synuclein | 113 | 5625 | 0.062 | 0.000 | 0.000 | 0.000 | 0.000 |
| | (G111-G132) | | 5626 | 0.057 | 0.000 | 0.000 | 0.000 | 0.000 |
| | | | 5627 | 0.058 | 0.000 | 0.000 | 0.000 | 0.000 |
| | | | Avg. | 0.059 | 0.000 | 0.000 | 0.000 | 0.000 |
| 6 | UBITh1-εk-kkk-α-Synuclein | 107 | 5542 | 0.078 | 0.000 | 0.000 | 0.000 | 0.000 |
| | (D121-D135) | | 5543 | 0.069 | 0.000 | 2.468 | 2.349 | 2.980 |
| | | | 5544 | 0.082 | 0.000 | 0.000 | 0.000 | 0.000 |
| | | | Avg. | 0.076 | 0.000 | 0.823 | 0.783 | 0.993 |
| 7 | UBITh1-εk-kkk-α-Synuclein | 111 | 5619 | 0.058 | 0.000 | 0.000 | 0.662 | 1.887 |
| | (E123-D135) | | 5620 | 0.056 | 0.000 | 2.892 | 3.138 | 2.910 |
| | | | 5621 | 0.062 | 0.000 | 0.000 | 1.321 | 0.000 |
| | | | Avg. | 0.059 | 0.000 | 0.964 | 1.707 | 1.599 |
| 8 | UBITh1-εk-kkk-α-Synuclein | 112 | 5622 | 0.058 | 0.000 | 2.878 | 2.959 | 3.059 |
| | (E126-D135) | | 5623 | 0.063 | 0.000 | 0.000 | 0.000 | 0.000 |
| | | | 5624 | 0.053 | 1.437 | 2.933 | 2.996 | 2.940 |
| | | | Avg. | 0.058 | 0.479 | 1.937 | 1.985 | 2.000 |

TABLE 10
Inhibition of α-Syn Aggregation by Antibodies from Animals Receiving α-Syn Peptide
Immunogen Constructs

| | | | Aggregation Inhibition (%) | | |
|---|---|---|---|---|---|
| | SEQ ID | | IgG (μg/ml) | | |
| Peptide description | NO | WPI | 0.05 | 0.5 | 5 |
| UBITh3-εK-KKK-α-Synuclein ₈₅₋₁₄₀ | 106 | 3 | 33 | 49 | 45 |
| | | 8 | 51 | 72 | 76 |
| | | 13 | 47 | 50 | 43 |
| UBITh3-εK-KKK-α-Synuclein ₉₁₋₁₄₀ | 105 | 3 | 40 | 42 | 54 |
| | | 8 | 65 | 75 | 92 |
| | | 13 | 56 | 41 | 55 |
| UBITh3-εK-KKK-α-Synuclein ₁₀₁₋₁₄₀ | 102 | 3 | 45 | 45 | 53 |
| | | 8 | 55 | 73 | 70 |
| | | 13 | 41 | 51 | 48 |
| UBITh3-εK-KKK-α-Synuclein ₁₁₁₋₁₄₀ | 101 | 3 | 36 | 40 | 49 |
| | | 8 | 66 | 60 | 59 |
| | | 13 | 77 | 66 | 70 |
| UBITh3-εK-KKK-α-Synuclein ₁₂₁₋₁₄₀ | 100 | 3 | 41 | 44 | 46 |
| | | 8 | 51 | 77 | 76 |
| | | 13 | 40 | 47 | 54 |
| UBITh3-εK-KKK-α-Synuclein ₁₂₆₋₁₄₀ | 99 | 3 | 49 | 54 | 48 |
| | | 8 | 65 | 50 | 63 |
| | | 13 | 110 | 73 | 84 |
| UBITh1-εK-KKK-α-Synuclein ₉₇₋₁₃₅ | 110 | 6 | 51 | 54 | 83 |
| | | 9 | 27 | 74 | 77 |
| | | 12 | 44 | 41 | 55 |
| UBITh1-εK-KKK-α-Synuclein ₁₀₁₋₁₃₅ | 109 | 6 | 105 | 98 | 68 |
| | | 9 | 70 | 65 | 95 |
| | | 12 | 57 | 76 | 85 |
| UBITh1-εK-KKK-α-Synuclein ₁₁₁₋₁₃₅ | 108 | 6 | 55 | 84 | 82 |
| | | 9 | 52 | 70 | 82 |
| | | 12 | 56 | 58 | 87 |
| UBITh1-εK-KKK-α-Synuclein ₁₂₁₋₁₃₅ | 107 | 6 | 29 | 38 | 51 |
| | | 9 | 42 | 48 | 69 |
| | | 12 | 87 | 64 | 64 |
| | | 15 | 74 | 74 | 76 |
| UBITh1-εK-KKK-α-Synuclein ₁₂₃₋₁₃₅ | 111 | 6 | 34 | 45 | 60 |
| | | 9 | 42 | 30 | 48 |
| | | 12 | 58 | 55 | 59 |
| | | 15 | 56 | 64 | 75 |
| UBITh1-εK-KKK-α-Synuclein ₁₂₆₋₁₃₅ | 112 | 6 | 17 | 45 | 54 |
| | | 9 | 49 | 49 | 59 |
| | | 12 | 58 | 68 | 56 |
| | | 15 | 70 | 76 | 62 |
| UBITh1-εK-KKK-α-Synuclein ₁₀₁₋₁₃₂ | 114 | 6 | 79 | 83 | 87 |
| | | 9 | 61 | 66 | 87 |
| | | 12 | 48 | 55 | 51 |
| UBITh1-εK-KKK-α-Synuclein ₁₁₁₋₁₃₂ | 113 | 6 | 43 | 46 | 57 |
| | | 9 | 24 | 57 | 46 |
| | | 12 | 28 | 44 | 51 |

TABLE 11
Assessment of Neuroprotective Capacity on α-Syn Aggregates-Driven Neurodegeneration
by Neurite Length Quantification Through High-Content Analysis using Antibodies from
Animals Receiving α-Syn Peptide Immunogen Constructs

| | | | Neurite Length (%) | | |
|---|---|---|---|---|---|
| | SEQ ID | | IgG (μg/ml) | | |
| Peptide description | NO | WPI | 0.05 | 0.5 | 5 |
| UBITh3-εK-KKK-α-Synuclein ₈₅₋₁₄₀ | 106 | 3 | 6 | 10 | 25 |
| | | 8 | 8 | 11 | 17 |
| | | 13 | 9 | 8 | 26 |
| UBITh3-εK-KKK-α-Synuclein ₉₁₋₁₄₀ | 105 | 3 | 17 | 4 | 29 |
| | | 8 | 9 | 14 | 15 |
| | | 13 | 14 | 12 | 12 |
| UBITh3-εK-KKK-α-Synuclein ₁₀₁₋₁₄₀ | 102 | 3 | 12 | 9 | 27 |
| | | 8 | 12 | 11 | 14 |
| | | 13 | 10 | 10 | 18 |
| UBITh3-εK-KKK-α-Synuclein ₁₁₁₋₁₄₀ | 101 | 3 | 13 | 16 | 21 |
| | | 8 | 12 | 18 | 31 |
| | | 13 | 10 | 15 | 23 |
| UBITh3-εK-KKK-α-Synuclein ₁₂₁₋₁₄₀ | 100 | 3 | 10 | 8 | 23 |
| | | 8 | 9 | 15 | 29 |
| | | 13 | 5 | 18 | 19 |
| UBITh3-εK-KKK-α-Synuclein ₁₂₆₋₁₄₀ | 99 | 3 | 13 | 24 | 26 |
| | | 8 | 12 | 24 | 48 |
| | | 13 | 12 | 17 | 35 |
| UBITh1-εK-KKK-α-Synuclein ₉₇₋₁₃₅ | 110 | 6 | 13 | 15 | 17 |
| | | 9 | 8 | 12 | 16 |
| | | 12 | 13 | 14 | 23 |
| UBITh1-εK-KKK-α-Synuclein ₁₀₁₋₁₃₅ | 109 | 6 | 9 | 10 | 12 |
| | | 9 | 12 | 8 | 17 |
| | | 12 | 13 | 10 | 12 |
| UBITh1-εK-KKK-α-Synuclein ₁₁₁₋₁₃₅ | 108 | 6 | 11 | 14 | 19 |
| | | 9 | 11 | 14 | 27 |
| | | 12 | 9 | 16 | 26 |
| UBITh1-εK-KKK-α-Synuclein ₁₂₁₋₁₃₅ | 107 | 6 | 15 | 22 | 31 |
| | | 9 | 13 | 17 | 34 |
| | | 12 | 11 | 16 | 26 |
| | | 15 | 9 | 16 | 15 |
| UBITh1-εK-KKK-α-Synuclein ₁₂₃₋₁₃₅ | 111 | 6 | 14 | 13 | 31 |
| | | 9 | 11 | 21 | 29 |
| | | 12 | 10 | 12 | 22 |
| | | 15 | 8 | 8 | 15 |
| UBITh1-εK-KKK-α-Synuclein ₁₂₆₋₁₃₅ | 112 | 6 | 13 | 26 | 55 |
| | | 9 | 13 | 20 | 46 |
| | | 12 | 12 | 12 | 22 |
| | | 15 | 11 | 10 | 14 |
| UBITh1-εK-KKK-α-Synuclein ₁₀₁₋₁₃₂ | 114 | 6 | 11 | 18 | 27 |
| | | 9 | 12 | 29 | 64 |
| | | 12 | 12 | 22 | 50 |
| UBITh1-εK-KKK-α-Synuclein ₁₁₁₋₁₃₂ | 113 | 6 | 10 | 15 | 31 |
| | | 9 | 14 | 26 | 55 |
| | | 12 | 14 | 21 | 59 |

TABLE 12
Neuroprotective Assessment in α-Syn Aggregates-Driven Neurodegenerative Neurons by
Neuron Number Quantification Through High-Content Analysis using Antibodies from
Animals Receiving α-Syn Peptide Immunogen Constructs

| | | | Neuron Survival (%) | | |
|---|---|---|---|---|---|
| | SEQ ID | | IgG (μg/ml) | | |
| Peptide description | NO | WPI | 0.05 | 0.5 | 5 |
| UBITh3-εK-KKK-α-Synuclein ₈₅₋₁₄₀ | 106 | 3 | 18 | 23 | 22 |
| | | 8 | 19 | 18 | 25 |
| | | 13 | 20 | 23 | 20 |
| UBITh3-εK-KKK-α-Synuclein ₉₁₋₁₄₀ | 105 | 3 | 26 | 29 | 31 |
| | | 8 | 22 | 27 | 31 |
| | | 13 | 24 | 23 | 21 |
| UBITh3-εK-KKK-α-Synuclein ₁₀₁₋₁₄₀ | 102 | 3 | 11 | 14 | 17 |
| | | 8 | 16 | 20 | 23 |
| | | 13 | 17 | 18 | 20 |
| UBITh3-εK-KKK-α-Synuclein ₁₁₁₋₁₄₀ | 101 | 3 | 23 | 21 | 31 |
| | | 8 | 20 | 34 | 43 |
| | | 13 | 24 | 26 | 28 |
| UBITh3-εK-KKK-α-Synuclein ₁₂₁₋₁₄₀ | 100 | 3 | 25 | 28 | 35 |
| | | 8 | 22 | 34 | 39 |
| | | 13 | 21 | 38 | 43 |
| UBITh3-εK-KKK-α-Synuclein ₁₂₆₋₁₄₀ | 99 | 3 | 25 | 32 | 41 |
| | | 8 | 22 | 37 | 42 |
| | | 13 | 16 | 28 | 25 |
| UBITh1-εK-KKK-α-Synuclein ₉₇₋₁₃₅ | 110 | 6 | 23 | 19 | 24 |
| | | 9 | 22 | 26 | 27 |
| | | 12 | 16 | 24 | 30 |
| UBITh1-εK-KKK-α-Synuclein ₁₀₁₋₁₃₅ | 109 | 6 | 18 | 23 | 27 |
| | | 9 | 25 | 21 | 22 |
| | | 12 | 22 | 26 | 29 |
| UBITh1-εK-KKK-α-Synuclein ₁₁₁₋₁₃₅ | 108 | 6 | 23 | 37 | 42 |
| | | 9 | 28 | 45 | 65 |
| | | 12 | 24 | 34 | 46 |
| UBITh1-εK-KKK-α-Synuclein ₁₂₁₋₁₃₅ | 107 | 6 | 19 | 26 | 49 |
| | | 9 | 24 | 22 | 31 |
| | | 12 | 19 | 26 | 28 |
| | | 15 | 20 | 19 | 22 |
| UBITh1-εK-KKK-α-Synuclein ₁₂₃₋₁₃₅ | 111 | 6 | 20 | 26 | 29 |
| | | 9 | 24 | 21 | 31 |
| | | 12 | 19 | 24 | 32 |
| | | 15 | 20 | 36 | 49 |
| UBITh1-εK-KKK-α-Synuclein ₁₂₆₋₁₃₅ | 112 | 6 | 20 | 36 | 49 |
| | | 9 | 26 | 30 | 35 |
| | | 12 | 28 | 30 | 36 |
| | | 15 | 25 | 20 | 31 |
| UBITh1-εK-KKK-α-Synuclein ₁₀₁₋₁₃₂ | 114 | 6 | 22 | 30 | 43 |
| | | 9 | 26 | 37 | 57 |
| | | 12 | 25 | 34 | 55 |
| UBITh1-εK-KKK-α-Synuclein ₁₁₁₋₁₃₂ | 113 | 6 | 24 | 34 | 34 |
| | | 9 | 22 | 39 | 50 |
| | | 12 | 21 | 31 | 38 |

TABLE 13
In Vivo Efficacy Study of α-Syn Peptide Immunogen Constructs
Administered to MPP⁺-Induced Parkinson Disease Mouse Model
MPP+ induced Balb/c mice model

| Week | −2 | 0 | 1 | 2 | 3 | 4 | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|---|---|---|---|
| Body Weight | | | | | | | | Δ | | Δ | Δ | | | | | | Δ | | | | |
| MPP+ ICV | Δ | | | | | | | | | | | | | | | | | | | | |
| Immunization | | ▴ | | | ▴ | | | ▴ | | | | | | | | | | | | | |
| Motor ability | Δ | | | Δ | | Δ | | | | Δ | | | | | | | | | | | |
| Venipuncture | Δ | | Δ | | | Δ | | | Δ | | | Δ | | Δ | | | | | | | Δ |
| Tissue harvest | | | | | | | | | | | | | | | | | | | | | Δ |

TABLE 14
In Vivo Efficacy Study of α-Syn Peptide Immunogen Constructs
Administered to α-Syn-Inoculated Parkinson Disease Mouse Model
Fibrillar α-Syn-inoculated FVB mice model

| Week | −7 | 0 | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|---|---|
| Body Weight | Δ | | Δ | | Δ | | Δ | | Δ | | Δ | | Δ | | Δ | | Δ |
| αSyn-inoculation | Δ | | | | | | | | | | | | | | | | |
| Immunization | | ▴ | | | ▴ | | | ▴ | | | | | | | | | |
| Motor ability | | | | | | | | | | Δ | | | | | | | |
| Venipuncture | Δ | | | Δ | | | Δ | | | Δ | | | Δ | | | | Δ |
| Tissue harvest | | | | | | | | | | | | | | | | | Δ |

TABLE 15
List of Cases obtained from UCL and Their Diagnosis Post-Mortem

| Case ID | Age | Gender | Diagnosis |
|---|---|---|---|
| PD505 | TBC | TBC | MSA |
| PD363 | TBC | TBC | MSA |
| PD300 | TBC | TBC | MSA |
| PD294 | TBC | TBC | DLB |
| PD330 | TBC | TBC | DLB |
| PD385 | TBC | TBC | DLB |
| PD451 | TBC | TBC | PD |
| PD458 | TBC | TBC | PD |
| PD413 | TBC | TBC | PD |
| PDC87 | TBC | TBC | CONTROL |

1. An alpha-synuclein (α-Syn) peptide immunogen construct comprising:
a B cell epitope comprising about 10 to about 25 amino acid residues from a C-terminal fragment of α-Syn corresponding to about amino acid G111 to about amino acid D135 of SEQ ID NO: 1;
a T helper epitope comprising an amino acid sequence selected from the group consisting of SEQ ID NOs: 70-98; and
an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 148),
wherein the B cell epitope is covalently linked to the T helper epitope directly or through the optional heterologous spacer.

2. The α-Syn peptide immunogen construct of claim 1, wherein the B cell epitope is selected from the group consisting of SEQ ID NOs: 12-15, 17, and 49-63.

3. The α-Syn peptide immunogen construct of claim 1, wherein the T helper epitope is selected from the group consisting of SEQ ID NOs: 81, 83, and 84.

4. The α-Syn peptide immunogen construct of claim 1, wherein the optional heterologous spacer is (α, ε-N)Lys or ε-N-Lys-Lys-Lys-Lys (SEQ ID NO: 148).

5. The α-Syn peptide immunogen construct of claim 1, wherein the T helper epitope is covalently linked to the amino terminus of the B cell epitope.

6. The α-Syn peptide immunogen construct of claim 1, wherein the T helper epitope is covalently linked to the amino terminus of the B cell epitope through the optional heterologous spacer.

7. The α-Syn peptide immunogen construct of claim 1 comprising the following formula:
(Th)ₘ-(A)ₙ-(α-Syn C-terminal fragment)-X
or
(α-Syn C-terminal fragment)-(A)ₙ-(Th)ₘ-X
wherein
Th is the T helper epitope;
A is the heterologous spacer;
(α-Syn C-terminal fragment) is the B cell epitope;
X is an α-COOH or α-CONH₂ of an amino acid;
m is from 1 to about 4; and
n is from 1 to about 10.

8. The α-Syn peptide immunogen construct of claim 1, comprising the amino acid sequence selected from the group consisting of SEQ ID NOs: 107, 108, 111-113, and 115-147.

9. The α-Syn peptide immunogen construct of claim 1, comprising the amino acid sequence selected from the group consisting of SEQ ID NOs: 107, 108, and 111-113.

10. A composition comprising the α-Syn peptide immunogen construct of claim 1.

11. A composition comprising more than one α-Syn peptide immunogen construct of claim 1.

12. The composition of claim 11, wherein the α-Syn peptide immunogen constructs have amino acid sequences of SEQ ID NOs: 112 and 113.

13. A pharmaceutical composition comprising the α-Syn peptide immunogen construct of claim 1 and a pharmaceutically acceptable delivery vehicle and/or adjuvant.

14. The pharmaceutical composition of claim 13, wherein
a. the α-Syn peptide immunogen construct is selected from the group consisting of SEQ ID NOs: 107, 108, 111-113, and 115-147; and
b. the adjuvant is a mineral salt of aluminum selected from the group consisting of Al(OH)₃ or AlPO₄.

15. The pharmaceutical composition of claim 13, wherein
a. the α-Syn peptide immunogen construct is selected from the group consisting of SEQ ID NOs: 107, 108, 111-113, and 115-147; and
b. the α-Syn peptide immunogen construct is mixed with an CpG oligodeoxynucleotide (ODN) to form a stabilized immunostimulatory complex.

16. An isolated antibody or epitope-binding fragment thereof that specifically binds to the B cell epitope of the α-Syn peptide immunogen construct of claim 1.

17. The isolated antibody or epitope-binding fragment thereof according to claim 16 bound to the α-Syn peptide immunogen construct.

18. An isolated antibody or epitope-biding fragment thereof that specifically binds to the B cell epitope of the α-Syn peptide immunogen construct of claim 9.

19. A composition comprising the isolated antibody or epitope-binding fragment thereof according to claim 16.

20. A composition comprising the isolated antibody or epitope-binding fragment thereof according to claim 18.

21. The composition of claim 20, comprising a mixture of
a. an isolated antibody or epitope-binding fragment thereof that specifically binds to the B cell epitope of SEQ ID NO: 112; and
b. an isolated antibody or epitope-binding fragment thereof that specifically binds to the B cell epitope of SEQ ID NO: 113.

22. A method of producing antibodies that recognize α-Syn in a host comprising administering to the host a composition comprising the α-Syn peptide immunogen of claim 1 and a delivery vehicle and/or adjuvant.

23. A method of inhibiting α-Syn aggregation in an animal comprising administering a pharmacologically effective amount of the α-Syn peptide immunogen of claim 1 to the animal.

24. A method of reducing the amount of α-Syn aggregates in an animal comprising administering a pharmacologically effective amount of the α-Syn peptide immunogen of claim 1 to the animal.

25. A method of identifying α-Syn aggregates of different sizes in a biological sample comprising:
a. exposing the biological sample to the antibody or epitope-binding fragment thereof according to claim 16 under conditions that allow the antibody or epitope-binding fragment thereof to bind to the α-Syn aggregates; and
b. detecting the amount of the antibody or epitope-binding fragment thereof bound to the α-Syn aggregates in the biological sample.
